(19) United States
(12) Patent Application Publication
(10) Pub. No.: US 2012/0101087 A1
(43) Pub. Date: Apr. 26, 2012

(54) Generation of Combinatorial Synthetic Libraries and Screening for Novel Proadhesins and Nonadhesins

(76) Inventors: Randall S. Alberte, Estero, FL (US); Robert D. Smith, Salinas, CA (US)

(21) Appl. No.: 12/334,094

(22) Filed: Dec. 12, 2008

Related U.S. Application Data
(60) Provisional application No. 61/013,269, filed on Dec. 12, 2007.

(51) Int. Cl. A01N 37/20; C07C 235/44; A01P 1/00; A01N 43/48; C07D 295/192; A01N 43/60; C07C 235/66; C07D 243/08

(52) U.S. Cl. 514/218; 564/172; 514/622; 564/179; 540/575; 564/169; 560/250; 514/546; 544/387; 514/255.01

(57) ABSTRACT

The present invention relates to, in part, novel bisphenol compounds useful for the recognition, attachment and growth of unwanted biologics on natural and manmade surfaces. Another aspect of the invention relates to combinatorial libraries for producing the same. Another aspect of the invention relates to pharmaceutical formulations comprising the same.

### RELATED APPLICATIONS

This application claims the benefit of priority to U.S. Provisional Application No. 61/013,269, filed on Dec. 12, 2007, which is hereby incorporated by reference in its entirety.

### BACKGROUND OF THE INVENTION

Infections caused by or related to microbial agents are a major cause of human illness worldwide, and the frequency of resistance to standard anti-infective agents has risen dramatically over the last decade. Infective agents include but are not limited to bacteria, viruses, fungi, and amyloid peptide based agents like prions.

For example, methicillin resistant Staphylococcus aureus (MRSA) have become a major public health concern. Increasing numbers of individuals, and particularly the young and elderly, test positive for MRSA strains of this Gram positive bacterium common to blood stream infections, cutaneous infections and medical device biofilms. Antibiotic resistance is also common in Gram negative bacteria including entercocci and Pseudomonas aeruginosa. The entercocci are causative agents of many gastrointestinal tract disorders, and stains of vancomycin-resistant Enterococcus faecalis and E. faecium (VRE) have become common in processed foods and meat, and in public bathing areas. Yesim Cetinkaya, Pamela Falk, and C. Glen Mayhall, 2000. Clin. Microbiol. Rev. 13:686-707). Pseudomonas aeruginosa infections of the upper respiratory tract is the major cause of morbidity and mortality in adult patients with cystic fibrosis (CF). Hoiby, N., and C. Koch. Thorax, 1990, 45:881-884. Recent advances in antiinfective therapy against lung pathogens have dramatically contributed to increased life expectancy of CF patients. However, frequent and prolonged antibiotic courses are likely to be a major factor in the selection of highly antibiotic-resistant P. aeruginosa strains. Similar resistance issues have arisen for human fungal pathogens. The resistance problems are enhanced in HIV patients and other individuals with compromised immune systems due to chemotherapy, organ transplants, and long-term hospitalization. MA. Ghannoum and LB. Rice. 1999. Antifungal Agents Mode of Action, Mechanisms of Resistance, and Correlation of These Mechanisms with Bacterial Resistance. Clin. Microbiol. Rev. 12:501-517.

A viral infection begins when a virion comes into contact with a host cell and attaches or adsorbs, to it. The viral (DNA or RNA) then crosses the plasma membrane into the cytoplasm and eventually enter into the nucleus. In the case of retrovirus, the viral RNA is reverse transcribed into DNA. Viral DNA is then integrated into the chromosomal DNA of the infected cell. Integration is mediated by an integration protein, integrase. All integrated proviruses are required for the subsequent transcription process which is acted upon by the host cell transcription factors. The integrated DNA is transcribed by the cell's own machinery into mRNA, or replicated and becomes enclosed in a virion. For retrovirus, the integrated DNA is transcribed into RNA that either acts as mRNA or become enclosed in a virion. This completes the virus life cycle.

Seasonal waves of influenza virus infections have caused over 36,000 deaths per year in the United States alone. Smith N M, Bresee J S, Shay D K, Uyeki T M, Cox N J, Strikas R A: Center for Disease Control and Prevention: Prevention and control of influenza: recommendations of the Advisory Committee on Immunization Practices (APIP). MMWR Recomm Rep 2006, 55:1-42. Less than 100 years ago, a single strain of H1N1 influenza virus caused a pandemic with more human fatalities than any other single infectious event, war, or famine in world history. Achievements in Public Health, 1900-1999: Control of Infectious Diseases. MMWR Morb Mortal Wkly Rep 1999, 48:621-629. More recently, a highly pathogenic H5N1 strain of avian influenza has been repeatedly transmitted from birds to humans, resulting in several hundred human deaths. Update: influenza activity—United States and worldwide, 2005-06 season, and composition of the 2006-07 influenza vaccine. MMWR Morb Mortal Wkly Rep 2006, 55:648-653; World Heath Organization: H5N1 avian influenza: timeline of major events [http://www.who.int/csr/disease/avian_influenza/timeline_{—}2007_{—}05_{—}10.pdf]. Fortunately, this has generated few cases of human-to-human transmission and has not yet resulted in a major human pandemic. Webster R G, Peiris M, Chen H, Guan Y: H5N1 outbreaks and enzootic influenza. Emerg Inf Dis 2006, 12:3-8; Nicholls J M, Chan M C W, Chan W Y, Wong H K, Cheung, C Y, Kwong L W, Wong M P, Chui, W H, Poon L L M, Tsao S W, Guan Y, Peiris, J S M: Tropism of avian influenza A (H5N1) in the upper and lower respiratory tract. Nature Med 2007, 13:147-149. It is clear that the natural influenza reservoir has the capacity to generate new virus strains that can cross species barriers and produce human infections with increased pathogenicity and in some cases increased human-to-human transmission characteristics. These strains present a real and potentially uncontrollable threat to global public health. Nelson, M I, Holmes, E C: The evolution of epidemic influenza. Nat Rev Genetics 2007, 8:196-205.

Influenza viruses are lipid enveloped, with segmented, negative-stranded RNA genomes. Webster R G, Bean W J, Gorman O T, Chambers T M, Kawaoka Y: Evolution and ecology of influenza A viruses. Microbiol Rev 1992, 56:152-179; Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579. They are capable of rapid evolution through the accumulation of point mutations as well as by re-assortment of RNA segments to generate novel progeny. Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579. The ecological cycles of influenza viruses include replication in a large and genetically diverse wild reservoir dominated by water birds as hosts. Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579. Viruses from this reservoir continually spill over into other avian and mammalian host populations, including humans. Hlinak A, Mühle R U, Werner O, Globig A, Starick E, Schirrmeier H, Hoffmann B, Engelhardt A, Hübner D, Conraths F J, Wallschlager D, Kruckenberg H, Müller T: A virological survey in migrating waders and other waterfowl in one of the most important resting sites of Germany. J Vet Med B Infect Dis Vet Public Health 2006, 53:105-110; Humberd J, Guan Y, Webster R G: Comparison of the replication of influenza A viruses in Chinese ring-necked pheasants and Chukar partridges. J Virol 2006, 80:2151-61; Perdue M L, Swayne D E: Public health risk from avian influenza viruses. Avian Dis 2005, 49: 317-327; Alexander D J, Brown I H: Recent zoonoses caused by influenza A viruses. Rev Sci Tech 2000, 19:197-225. Survivors of influenza virus infection generally mount an immune response with only limited cross-reactivity to other influenza strains, resulting in multiple infections throughout an individual's life time (Couch R B: An overview of serum antibody responses to influenza virus antigens. Dev Biol (Basel) 2003, 115:25-30), and multiple epidemics and pandemics (Kilbourne E D: Influenza pandemics of the 20th century. Emerg Infect Dis 2006, 12:9-14) when previously exposed populations are confronted with new virus strains. Alexander D J, Brown I H: Recent zoonoses caused by influenza A viruses. Rev Sci Tech 2000, 19:197-225; Influenza vaccines. Wkly Epidemiol Rec 2005, 80:277-288; Webster R G: Immunity to influenza in the elderly. Vaccine 2000, 18:1686-1689.

Current influenza control efforts have concentrated on the use of vaccines and a small number of anti-influenza drugs. Because influenza vaccines are only partially cross-protective, they must be developed and produced de novo each year, based on predictions of which strains are likely to circulate the following year. Recommended composition of influenza virus vaccines for use in the 2007 influenza season. Wkly Epidemiol Rec 2006, 81:390-395. This prevents stockpiling and use of vaccination distribution strategies to control future severe outbreaks. Two main classes of anti-influenza drugs have been developed and are in current use. Inhibitors of the viral ion channel M protein, such as amantidine (Davies W L, Grunert R R, Haff R F, McGahen J W, Neumayer E M, Paulshock M, Watts J C, Wood R T, Hermann E C, Hoffmann C E: Antiviral activity of 1-adamantanamine (amantadine). Science 1964, 144:862-863; Shimbo K, Brassard D L, Lamb R A, Pinto L H: Ion selectivity and activation of the M2 ion channel of influenza virus. Biophys J 1996, 70:1335-1346) and rimantidine (Rabinovich S, Baldini J T, Bannister R: Treatment of influenza. The therapeutic efficacy of rimantadine HC1 in a naturally occurring influenza A2 outbreak. Am J Med Sci 1969, 257:328-335; Chizhmakov I V, Geraghty F M, Ogden D C, Hayhurst A, Antoniou M, Hay A J: Selective proton permeability and pH regulation of the influenza virus M2 channel expressed in mouse erythroleukaemia cells. J Physiol pt 2 1996, 494:329-336), have been produced and commercialized, as well as have inhibitors of the viral surface neuraminidase enzyme, such as oseltamivir (Kati W M, Saldivar A S, Mohamadi F, Sham H L, Laver W G, Kohlbrenner W E: GS4071 is a slow-binding inhibitor of influenza neuraminidase from both A and B strains. Biochem Biophys Res Commun 1998, 244:408-413), which is now in wide use. These drugs are effective as prophylactics in blocking the development of influenza virus symptoms (Parker R, Loewen N, Skowronski D: Experience with oseltamivir in the control of a nursing home influenza B outbreak. Can Commun Dis Rep 2001, 27:37-40) as well as therapeutically treating (Kawai N, Ikematsu H, Iwaki N, Maeda T, Satoh I, Hirotsu N, Kashiwagi S: A Comparison of the effectiveness of oseltamivir for the treatment of influenza A and influenza B: A Japanese multicenter study of the 2003-2004 and 2004-2005 influenza seasons. Clin Infect Dis 2006, 43:439-444), and reducing the duration of symptoms post-infection. Gillissen A, Höffken G: Early therapy with the neuraminidase inhibitor oseltamivir maximizes its efficacy in influenza treatment. Med Microbiol Immunol 2002, 191:165-168. However, due to the ability of influenza viruses to rapidly mutate, drug resistance against each of the antiviral classes has appeared quickly. Smith N M, Bresee J S, Shay D K, Uyeki T M, Cox N J, Strikas R A: Center for Disease Control and Prevention: Prevention and control of influenza: recommendations of the Advisory Committee on Immunization Practices (APIP). MMWR Recomm Rep 2006, 55:1-42; Moscona A: Neuraminidase inhibitors for influenza. New Engl J Med 2005, 353:1363-137327. Today, the M protein inhibitors, amantidine and rimantidine, are no longer in common use in many areas because of viral resistance, just a few years after their commercial distribution. Saito R, Sakai T, Sato I, Sano Y, Oshitani H, Sato M, Suzuki H: Frequency of amantadine-resistant influenza A viruses during two seasons featuring cocirculation of H1N1 and H3N2. J Clin Microbiol 2003, 41:2164-2165. Resistance to oseltamivir has also been reported in human and avian influenza viruses, and is predicted to increase with increased usage. Guberava L V, Kaiser L, Matrosovich M N, Soo-Hoo Y, Hayden F G: Selection of influenza virus mutants in experimentally infected volunteers treated with oseltamivir. J Infect Dis 2001, 183:523-531; Kiso M, Mitamura K, Sakai-Tagawa Y, Shiraishi K, Kawakami C, Kimura K, Hayden F G, Sugaya N, Kawaoka Y: Resistant influenza A viruses in children treated with oseltamivir: descriptive study. Lancet 2004, 364:759-765. New anti-influenza drugs will be required to keep pace with the ability of influenza viruses to mutate and develop resistance to current drugs.

The discovery of AZT as an effective disrupter of the HIV-1 viral cycle has improved the quality of life and extended the lives of many HIV positive individuals, though often with significant side effects. Unfortunately, regular use of AZT and other viral reverse transcriptase inhibitors, HIV proteases inhibitors, and Highly Active Antiretroviral Therapy (HAART) that involves multi-drug therapies has lead to the generation of resistant HIV strains, making control of HIV viral load in HIV+ and AIDS patients more difficult. The development of enfuvirtide (also termed T-20 or Fuzeon®), which controls HIV strains resistant to nucleosides, non-nucleosides, nucleotides, and protease inhibitors, through blocking viral fusion, was a significant advancement in HIV treatments because it addressed a new therapeutic target. Although very effective, there are major drawbacks that limit its compliance and use in non-clinical settings. The need for new HIV therapies that have novel therapeutic targets is well recognized and is an imperative for this global public health problem.

In the past two decades, the emergence of human immunodeficiency virus type 1 (HIV-1), Human Influenza (H1N1), Avian Flu (H5N1), Dengue, and West Nile virus as an important human pathogens has led to a resurgence of scientific interest in retroviruses and other viruses. Unfortunately, for viruses like Dengue, there are no known treatments and the numbers of cases world-wide are increasing dramatically, with significant northern latitude expansion of infection due to the northern drift of the Aedes aegypti mosquito, the insect host for Dengue viruses. Current antivirals target, for the most part, various steps in the viral replication cycle, and resistance to these agents is significant, particularly with patients with HIV-1 infections. Pillay D. 1998. Emergence and control of resistance to antiviral drugs in resistance in herpes viruses, hepatitis B virus, and HIV. Commun Dis Public Health 1:5-13; Larder B A. 1996. Nucleoside and foscarnet-mechanisms. In: Richman D D, ed. Antiviral Drug Resistance. London: Wiley, pp. 169-190. Other viruses like Rhinoviruses, a major cause of the common cold, though not classified as envelop viruses share many surface properties with envelope viruses like influenza Rhinoviruses have distinct Glycosylated capsid proteins that have been shown to be responsible for host cell binding and entery. The primary route of entry for rhinoviruses is the upper respiratory tract. The virus binds to ICAM-1 (intracellular adhesion molecule-1) receptors on respiratory epithelial cells. As the virus replicates and spreads, infected cells release distress signals known as chemokines and cytokines (which in turn activate inflammatory mediators). These events lead to the typical symptoms of the common cold. Smith T, Kremer M, Luo M, Vriend G, Arnold E, Kamer G, Rossmann M, McKinlay M, Diana G, Otto M (1986). The site of attachment in human rhinovirus 14 for antiviral agents that inhibit uncoating. Science 233 (4770):1286-93. There is been no success in developing a vaccine or drugs effective against this class of viruses despite that fact the colds result in billions of dollars yearly in loss productivity and over 22 million lost school days for children in the US alone (US CDC).

Prions, or proteinaceous infectious particles, are the cause of a number transmissible of neurodegenerative diseases in mammals that include bovine spongiform encephalopathies (BSE) (Westaway, D, Telling, G. and Priola, S. 1998. Prions. Proc. Natl. acad. Sci. USA 95:11030-11031. In the mid 1980's, over 200,000 cases of BSE were reported, though human cases are much lower. Belay, E. D. and Schonberger, L. B. 2005. The Public health impact of Prion diseases. Annu. Rev. Public Health 26:191-212. Prions are malformed proteins that form plaques or amyloids on cerebral neuronal tissues leading to disruption of neuron function and apoptosis. Amyloids is a general term for protein fragments that the body produces normally, and in the case of prions, the amyloids are proteins with an aberrant folding or conformation. There are no current treatments for these progressive disorders or drugs that prevent amyloid generation and deposition.

Though not infectious agents, amyloids derived from protelytic processing of a amyloid precursor protein (APP), a type I transmembrane protein, that yield small 40- and 42-amino acid peptides terms Aβ1₁₋₄₂ and related peptides, are the primary cause of dementia and Alzheimer's Disease. The Aβ₄₂ and related peptides aggregate within and on neuronal cells and form plaques that deposit on the surface of neuronal cells and accumulate within the cells One of the hallmarks of Alzheimer's disease is the accumulation of oligimerized amyloid plaques between nerve cells (neurons) in the brain, the formation of tangled fibrils composed of amyloid polymers, and the accumulation of polymeric forms of the amyloids into hard insoluble plaques on brain tissues (LaFeria, F. M., Green, K. N. and Oddo, S. 2007. Nature Revs. Neurosci. 8:49-509). In a healthy brain, these protein fragments would be broken down and eliminated, but in the disease state the amyloids accumulate within and on neuronal cells leading to a cascade of oxidative stress processes, cell metabolism impairments, and cell death. These cellular events are clinically manifested initially in episodic memory loss but then progress to permanent memory loss, disorientation, significant loss in brain mass and ultimately to death. It is believed that drugs targeting amyloid aggregation, oligimerizationa, accumulation and deposition are key to therapeutic and prophylactic treatments.

Plants are constantly challenged by a wide variety of pathogenic organisms including fungi, viruses, and bacteria. Attempts have been made to control plant disease by means of disinfections, replacement of the soil, various cultural practices, genetic engineering of the plant, and control by chemicals. Some plants suffer from detrimental soil-spread diseases, which have not been possible to control owing to restrictions of use of chemical control agents and hazard periods due to possible residues or lack of sufficiently effective products. Extensive use of a broad range of anti-fungal agents on crops has lead to increasing rates of resistance, and current resistance to potato blight and soybean rust pathogens may have significant impacts of global food production. Eds. H. Lyr, P. E. Russell & H. D. Sisler. 1996. Modern fungicides and antifungal compounds. Intercept Ltd, Andover, Hants, 578 pp.

Protozoa and related eukaryotic parasites are major causes of disease including malaria, Giardia and other water-borne protozoans, certain sexually transmitted diseases, sleeping sickness (Trypanosomiasis), Leishmania, and a host of worm parasites. Quellette, M. 2001. Biochemical and molecular mechanisms of drug resistance in parasites. Trop. Med. Internatl. Health 60:874-882; White, N J. 2004. Anti-malarial drug resistance. J. Clin. Internatl. 110:1084-1092. It has been estimated that at least one-third of the world's human population is threatened by protozoan parasites. Resistance to such anti-protozoan drugs such as the sulfonamides, Chloroquine, Benimadazole, and Ivermectin is found world-wide and rates of resistance are increasing at an alarming rate. New drug targets, modes-of-action, and combination of drugs for anti-protozoan drugs are desperately needed that can not only overcome rapid resistance generation, but that minimize side effects and are cost effective. In nutrient limited ecosystems, bacteria have a marked tendency to adhere to surfaces and initiate the formation of a biofilm. A biofilm is a community of microbes, embedded in an organic polymer matrix, adhering to a surface. In nutrient limited natural and industrial ecosystems biofilm cells will predominate and cause problems as increased frictional resistance to fluids in water conduits and on ship hulls (fouling), decreased heat transfer from heat exchangers, corrosion of metallic substrata and contamination in the food and biotechnology industry. Biofilms are also a severe problem in medical science and industry causing dental plaque, contaminated endoscopes and contact lenses, prosthetic device colonization and biofilm formation on medical implants. More specifically, biofilms cause problems in a variety of areas including the bodies of humans and animals, food processing, health care facilities, metal-working shops, dairy fares and other industries.

Bacteria growing in biofilms are more resistant to antibiotics and disinfectants than planktonic cells and the resistance increases with the age of the biofilm. Bacterial biofilm also exhibits increased physical resistance towards desiccation, extreme temperatures or light. As mentioned, biofilm formation causes industrial, environmental and medical problems and the difficulties in cleaning and disinfection of bacterial biofilm with chemicals is a major concern in many industries. Furthermore, the trend towards milder disinfection and cleaning compositions may increase the insufficient cleaning of surfaces covered with biofilm.

In fact, one of the apparent purposes of natural biofilm formation is for the protection of the constituent microbes from a hostile environment. Consequently, there is a combative interaction between microbes in biofilms and biocidal vehicles such as preservatives, disinfectants and antibiotics. Further, the sessile mode of bacterial growth in biofilms differs from that of the same bacteria species that are present as planktonic cells in a circulating aqueous medium which interfaces with the biofilm. Biofilms also act as a trap for nutrient acquisition, which is an important factor when bacteria grow on surfaces and the nutrient supply is oligotrophic.

Biofilms consist of both host microbes and their extracellular products, usually exopolysaccharides. Microbes have a tendency to form these protective exopolysaccharide matrices after they have adhered to a surface. The formation of biofilm complexes requires only humid conditions and/or water systems and contact with a support surface and/or interface. With respect to nutrients, a nutrient deficiency in fact may increase the biofilm formation capacity of microbes. See 29 Adv. Appl. Microbiol. 93 (1983).

In general, biofilms can be produced by almost all microbes under suitable conditions. Various bacteria have been detected in biofilms, including several bacteria having pathogenic potential, such as Flavobacterium, Moraxella, Achromobacter, Pseudomonas, Alcaligenes, Micrococcus and Legionella. All of these bacteria have the potential to cause infections in humans, and Legionella, which is highly resistant to antibiotics, is of particular concern since infection can be fatal. The most common biofilm producers belong to the genera Pseudomonas, Enterobacter, Flavobacterium, Alcaligenes, Staphylococcus, and Bacillus. There also are anaerobes that can construct corrosive biofilms.

Bacterial deposits on the surfaces of food processing equipment can lead to potential contamination of food products. Bacterial deposits on medical devices such as implants, catheters and intraocular lenses also present the potential for infection when in contact with patients and medical personnel. Consequently, good hygiene standards require that food processing equipment be thoroughly cleaned between uses and that medical and dental equipment be sterilized if possible or at least, for non-surgical instruments, thoroughly cleaned before use. Other surfaces proximate to food processing or medical equipment, such as flooring, walls, tiles, conveyor belts, drains, and packaging, also should be regularly cleaned. Nonetheless, the potential always exists for residual bacterial deposits or for the incidental growth of bacteria on cleaned surfaces, particularly after contact with biological materials as encountered in food processing, e.g., blood, fats, and proteins. For equipment that cannot be thoroughly sterilized between uses, such as most food processing equipment, the potential exists for residual bacteria surface deposits because of the tenacious adherence of bacterial biofilms to the material of the processing equipment, commonly stainless steel but also often including other metals and polymers. Biofilms are communities of microorganisms adhering to surfaces of substrates, usually within a matrix of extracellular polymeric substances. Contamination of biological origin (hereafter biological contamination) on surfaces can include biofilms, blood, proteins, fats, oils, or combinations of such materials.

Besides causing problems in cleaning and hygiene, biofilms can cause energy losses and blockages in condenser and heat exchange tubes, interfere with water and waste water systems, and form drag-inducing encrustations on ship hulls. In the medical disciplines, a biofilm (referred to as “glycocalyx”) formed by bacteria such as a Pseudomonas species can be the systemic causation of diseases of the lungs or the gastrointestinal and urinary tracts. Additionally, a biofilm formed by bacteria such as Staphylococcus species can be a serious contamination problem in foreign-body instruments such as cardiac pacemakers, catheters, prostheses, artificial valves, and the like. Dental plaque is also a typical form of biofilm.

In many industrial processes and operations, for example cooling towers, heat exchangers, and paper manufacturing to name a few, microbials and biofilms lead to rapid deterioration of systems and compromise efficiencies and functions. Many commonly used biocidal agents have created unacceptable environmental risks, many are ineffective against microbial biofilms, some cause direct deterioration of certain systems like enhanced metal corrosion, and in some cases resistance to known treatments has been identified. Flemmng H-C. 2004. Biofouling in water systems—cases, causes and countermeasures. Appl. Microbiol. Biotech. 59:629-640; McDonnell, G and Russell, A D. 2002. Appl. Microbiol. Rev. 92:1 S-3S.

Other industrial systems are impacted by the attachment and growth of biological organism on surfaces. These include the diverse biofouling communities the form on manmade materials exposed to natural water systems. Biofouling on ship and boat hulls increases drag and can dramatically increase fuel consumption by upwards of 80% on hulls. Alberte, R. S. Snyder, S. Zahuranec, B. and Whetstone, M. 1992. Biofouling research needs for the United States Navy: Program history and goals. Biofouling 6:91-95. Ship hull fouling not only increases fuel consumption, but increases ship transit times and hence a lost revenues, increases maintenance costs, and contributes significantly (e.g., 10-20% of the global atmospheric carbon load) to global carbon emissions. The ability to mitigate and control biofouling will have major economic consequences, particularly if this can be accomplished using new technologies that are not based on toxic metals and biocidal activities.

The present invention provides new means to address the recognition, attachment and growth of unwanted biologics on natural and manmade surfaces.

### SUMMARY OF THE INVENTION

In a further embodiment, the present invention relates to a compound of formula 1a:

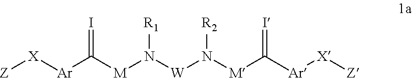

wherein, independently for each occurrence,
X and X′ represent independently O or S;
Z and Z′ each represent independently H, or optionally substituted alkyl, alkenyl, alkynyl, aryl, heteroaryl or acyl;
L and L′ each independently represent O, NR″, or S;
M and M′ independently represent a bond, or a bivalent alkyl or alkenyl chain.
W represents an optionally substituted bivalent alkyl, alkenyl, alkynyl, cycloalkyl, cycloalkenyl, aryl or heteroaryl group, wherein the alkyl, alkenyl or alkynyl groups optionally contain one or more heteroatoms selected from O, S, or NR″′;
R₁ and R₂ represent H, or optionally substituted alkyl, alkenyl, alkynyl, aralkyl, cycloalkyl, aryl or heteroaryl; or R₁ and R₂ may be joined together to form an optionally substituted 4 to 8 membered heterocyclic ring, wherein the ring includes W and the nitrogens to which R₁ and R₂ are attached, provided that the heterocyclic ring is not unsubstituted piperazine, and provided that when R₁ and R₂ are both H, W is not bivalent n-propyl;
Ar and Ar′ independently represent optionally substituted aryl or heteroaryl;
R′, R″ and R″′ represent independently for each occurrence H, formyl, sulfonyl, or —(CH₂)ₘ—R₈₀, or optionally substituted alkyl, alkenyl, aryl, aralkyl, formyl or acyl;
R₈₀ represents independently for each occurrence optionally substituted aryl, cycloalkyl, cycloalkenyl, or heterocyclyl; and
or a pharmaceutically acceptable salt thereof.

In another embodiment, the present invention relates to compounds of formula 1b,

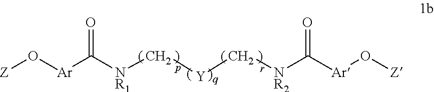

wherein, independently for each occurrence:

Y represents CH₂ or NR′;

R₁ and R₂ independently represent H, or optionally substituted alkyl or aryl, or R₁ and R₂ are joined together to form a optionally substituted 4 to 8 membered heterocyclic ring, wherein the ring includes —(CH₂)ₚ(Y)_{q}(CH₂)ᵣ— and the nitrogens to which R₁ and R₂ are attached; provided that the heterocyclic ring is not unsubstituted piperazine, and provided that when R₁ and R₂ are both H, W is not bivalent n-propyl;

p and r are 1, 2, or 3;

q is 0 or 1; and

Ar and Ar′ represent optionally substituted phenyl or substituted naphthyl;

Z and Z′ represent independently H or optionally substituted alkyl, alkenyl, alkynyl, aryl, heteroaryl or acyl;

or a pharmaceutically acceptable salt thereof.

In a further embodiment, the compound is:

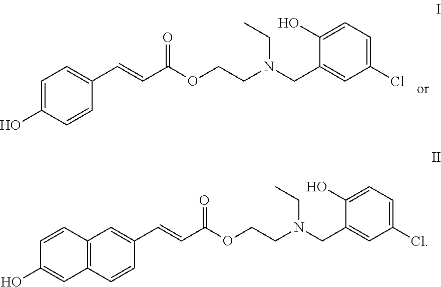

In another embodiment, the compound is selected from the group consisting of:

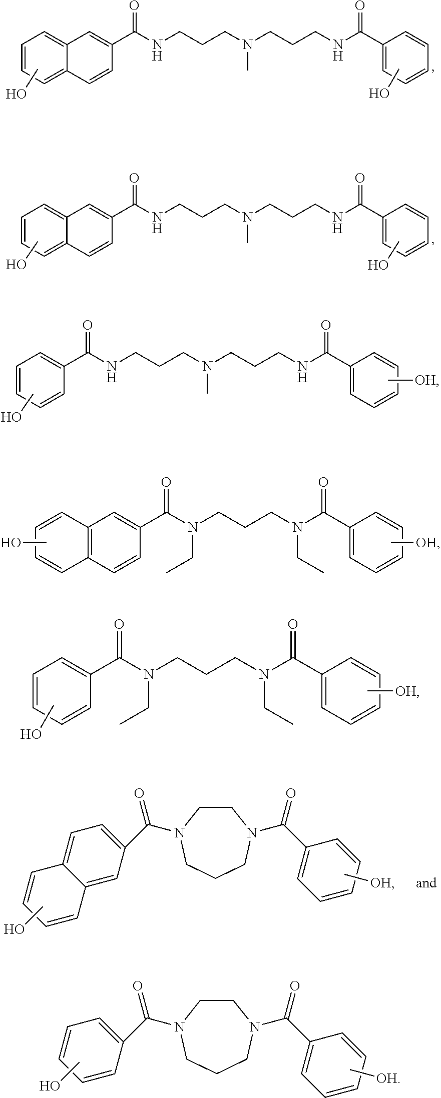

In another embodiment, the compound is:

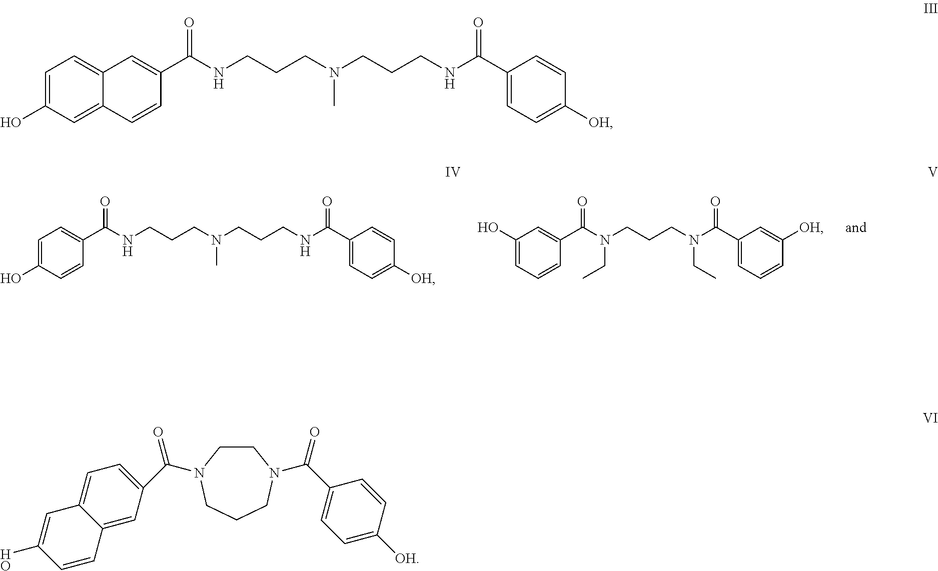

In certain embodiments, the present invention relates to a combinatorial library consisting of a plurality of compounds of the present invention.

In another embodiment, the present invention relates to a composition comprising a compound of the present invention, or combinations thereof. In a further embodiment, the composition further comprises a pharmaceutically acceptable carrier.

In another embodiment, the present invention relates to a combinatorial library comprising a plurality of compounds of the present invention.

These embodiments of the disclosure, other embodiments, and their features and characteristics, will be apparent from the description, drawings and claims that follow.

### BRIEF DESCRIPTION OF THE DRAWINGS

FIG. 1 depicts the basic structure of a compound comprising two sulfoxy ester moieties.

FIG. 2 depicts a strategy for solid phase synthesis of sulfoxy ester compounds.

FIG. 3 depicts a strategy for screening combinatorial libraries for novel nonadhesions.

FIG. 4 depicts a percent inhibition versus concentration graph for the effect of compound 1 on Human Influenza A (H1N1) showing an IC₅₀ of 155 μM.

FIG. 5 depicts a percent inhibition versus concentration graph for the effect of compound 4 on Human Influenza A (H1N1) showing an IC₅₀ of 66 μM.

FIG. 6 depicts the direct binding of I to Dengue virus. When this compound is bound Dengue infection is inhibited. Arrow indicates the DART-MS peak of compound I.

FIG. 7 depicts the direct binding of Ito Staphylococcus areus showing that cmp binds to the bacteria, and when bound blocks adhesion. Arrow indicates the DART-MS peak of compound I.

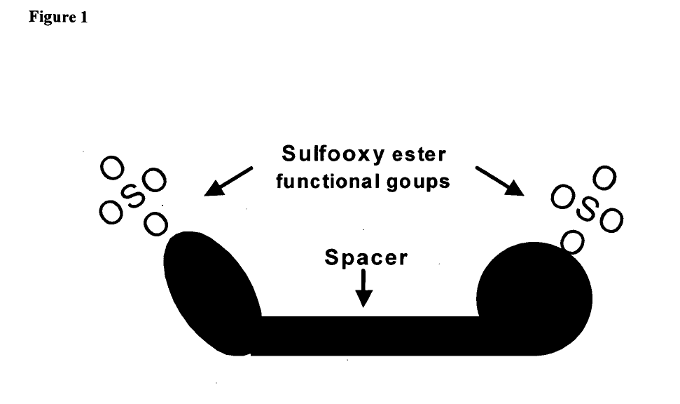

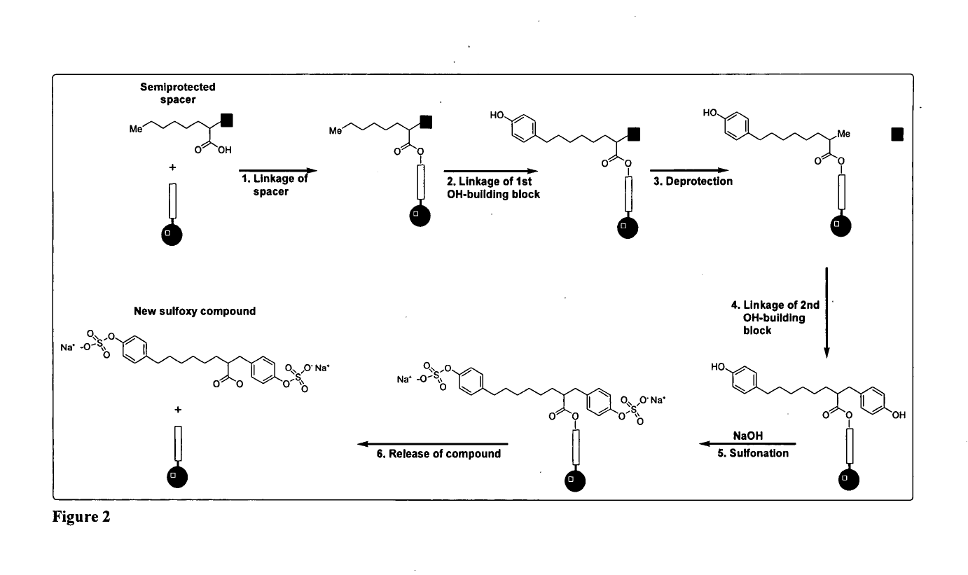

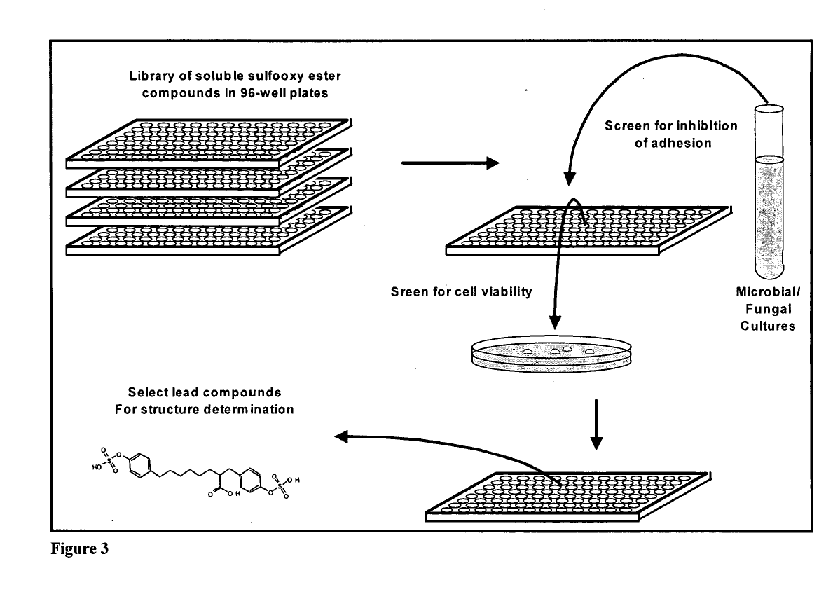

### DETAILED DESCRIPTION OF THE INVENTION

### Definitions

For convenience, certain terms employed in the specification, examples, and appended claims are collected here.

The term “surface” as used herein relates to any surface which may be covered by a biofilm layer. Examples of surfaces may be any hard surface such as metal, plastics, rubber, board, glass, wood, paper, concrete, rock, marble, gypsum and ceramic materials which optionally are coated, e.g. with paint, enamel etc.; or any soft surface such as fibers of any kind (yarns, textiles, vegetable fibers, rock wool, hair etc.); or porous surfaces; skin (human or animal); keratinous materials (nails etc.). The hard surface can be present in a process equipment member of a cooling tower, a water treatment plant, a dairy, a food processing plant, a chemical or pharmaceutical process plant. The porous surface can be present in a filter, e.g. a membrane filter.

The term “biofilm” refers to an accumulation of organisms on a surface. A mature biofilm can comprise a colony of microorganisms resident upon a surface surrounded by an exopolysaccharide.

“Biofouling organisms” refer to organisms or cells thereof that attach to surfaces and cause or contribute to biofouling or biofilm formation. These organisms can live in a wide range of environments, but most typically are in contact with a liquid or otherwise moist or humid environment. Biofouling organisms can live in either high or low flow aqueous environments, over a broad range of temperatures and pHs. Included are natural and man-made aquatic environments, including salt water, fresh water and brackish water. Also included are semi-aqueous or periodically aqueous environments which expose surfaces to high humidity, precipitation, or periodic flooding. Biofouling organisms can attach to surfaces at various stages of their life cycle, including larval stages, as well as adult. Examples of biofouling organisms include: macrofoulers, such as polychaetes colonial tunicates, and other sessile invertebrates including barnacles, bacteria, (gram negative and gram positive), algae, protists and fungi.

“Biofilm resistant” or “antifouling” refers to inhibition of attachment and/or growth of a biofouling organism.

A “biofilm or biofilm resistant coating” refers to any coating or surface that inhibits attachment and/or growth of biofouling organisms

The term “biologic” as used herein refers to any biofouling organism or any other organism capable of establishing an infection or otherwise causing pathological change in a host organism.

The term “nonadhesion” as used herein refers to any molecule that inhibits adhesion of biologics to surfaces.

The term “proadhesion” as used herein refers to any molecule that enhances adhesion of biologics to surfaces.

The term “aquatic vessel” as used herein refers to any vehicle or container at least part of which is submerged in water or an aqueous solution during its use or storage.

The term “off-shore platform” as used herein refers to any structure floating on the surface of or at least partly immersed in a body of water and secured to the floor of the body of water by legs extending from the structure to the floor.

The term “harbor infrastructure” as used herein refers to facilities provided in a port. Such facilities include, for example, docks, mooring poles, anchorage points, and other facilities known to one of ordinary skill in the art.

The term “conduit” as used herein refers to any channel through which a fluid is conveyed, said channel may comprise a filter. Such channels include, for example, a pipe, a tube, a hose, a faucet, a water storage device such as a water tower, and any other channel known to one of ordinary skill in the art.

The term “cable” as used herein refers to any assembly of fiber strands, wire, or metal links twisted or braided together or around a core, or any assemble of electrical conductors insulated from each other but laid up together usually by being twisted around a central core, or any other structure known to one of ordinary skill in the art.

The term “laboratory apparatus” as used herein refers to any structure, device, or article that may be used in a laboratory, especially any structure, device, or article that may be exposed to or submerged at least partly in water or an aqueous solution during its use or storage. Examples of such apparatus include water baths, glassware, plasticware, heating elements, incubators, and any other structures, devices, and articles known to one of ordinary skill in the art.

The term “virion” as used herein refers to any complete virus particle that consists of an RNA or DNA core with a protein coat and optionally with at least one external envelope.

The term “virion protein coat” as used herein refers to any capsid.

The term “virion envelope” as used herein refers to any external membrane of a virion and comprises a phospholipid bilayer and optionally at least one glycoprotein.

The term “implantable medical device” as used herein refers to any device that may be placed within the confines of an organism, either temporarily or permanently. Examples of implantable medical devices include pins, screws, plates, artificial joints and components thereof, artificial limbs and prostheses, sutures, shunts, artificial heart valves, artificial pacemakers and defibrillators, infusion pumps, vascular graft prostheses, tissue expanders, any other device known to one of ordinary skill in the art.

The term “insertable medical device” as used herein refers to any device that may be applied to the surface of an organism or placed partially inside an organism through a naturally existing or artificially created site of entry. Examples of such devices include contact lenses, stoma appliances, an artificial larynx, endotracheal tube, nasogastric tube, gastrostomy tube, jejunostomy tube, ileostomy tube, tracheostomy tube, intravenous catheter, Hickman catheters, vascular access device, transhepatic catheters, biliary drainage tube, urinary catheter, dialysis catheter, tympanostomy tubes, and any other devices known to one of ordinary skill in the art.

The term “food processing surface” as used herein refers to any surface used in the isolation, washing, cleaning, draining, manufacture, preparation, mixing, blending, serving, disposal, or storage of any material provided for, intended for, or being evaluated for digestive consumption by a human or non-human animal, or in any other step of food processing known to one of ordinary skill in the art. Example of such surfaces include tables, countertops, blenders, mixers, food processors, mixing bowls, serving bowls, sinks, wastebaskets, surfaces inside refrigerators and freezers, any utensils coming in contact with the material prepared, and any other surfaces recognized by one of ordinary skill in the art.

The term “ED₅₀” means the dose of a drug which produces 50% of its maximum response or effect. Alternatively, the dose which produces a pre-determined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀.

The term “structure-activity relationship (SAR)” refers to the way in which altering the molecular structure of drugs alters their interaction with a receptor, enzyme, etc.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “small molecule” is art-recognized and refers to a composition which has a molecular weight of less than about 2000 amu, or less than about 1000 amu, and even less than about 500 amu.

The term “acyl” as used herein refers to the radical

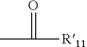

wherein R′₁₁ represents a hydrogen, an alkyl, an alkenyl or —(CH₂)ₘ—R₈, wherein R₈ is aryl, cycloalkyl, cycloalkenyl, or heterocyclyl; and m is an integer in the range 0 to 8, inclusive.

The term “alkyl” refers to a radical of saturated aliphatic groups, including straight-chain alkyl groups, branched-chain alkyl groups, and cycloalkyl-substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The terms “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxy, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

The term “cycloalkyl” as used herein refers to a radical of a saturated alicyclic, including polycyclic (e.g., bicyclic and tricyclic), and alkyl substituted cycloalkyl groups of 3 to 20 carbon atoms, preferably 3 to 10 carbon atoms, and more preferably, 5, 6 or 7 carbons in the ring structure.

As used herein, the term “nitro” means —NO₂; the term “halogen” designates —F, —Cl, —Br or —I; the term “sulfhydryl” means —SH; and the term “hydroxyl” means —OH.

The terms “amine” and “amino” are art-recognized and refer to both unsubstituted and substituted amines, e.g., a moiety that can be represented by the general formula:

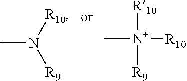

wherein R₉, R₁₀ and R′₁₀ each independently represent a group permitted by the rules of valence.

The term “acylamino” is art-recognized and refers to a moiety that can be represented by the general formula:

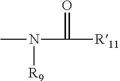

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

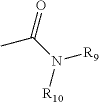

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

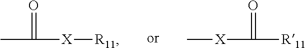

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

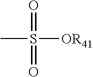

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

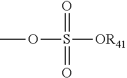

in which R₄₁ is as defined above.

The term “sulfonylamino” is art recognized and includes a moiety that can be represented by the general formula:

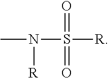

The term “sulfamoyl” is art-recognized and includes a moiety that can be represented by the general formula:

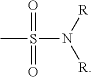

The term “sulfonyl”, as used herein, refers to a moiety that can be represented by the general formula:

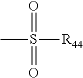

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

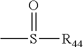

in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the term “substituted” is contemplated to include all permissible substituents of organic compounds. Illustrative substituents include, for example, those described herein above and as follows. The aforementioned groups may be optionally substituted with one or more substituents, where allowed by the rules of valence. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogen, trifluoromethoxy, trifluoromethyl, aralkyl, alkenyl, alkynyl, aryl, carboxyalkoxy, carboxyalkyl, cycloalkyl, cycloalkylalkyl, heterocyclyl, alkylheterocyclyl, heterocyclylalkyl, oxo, arylsulfonaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)—. For example, alkyl, alkenyl, alkynyl, aryl, cycloalkyl, heterocyclyl, acyl, amino, amido, etc. may be optionally substituted. In some embodiments, aforementioned groups may be optionally substituted with halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, or sulfonamido.

As used herein, the definition of each expression, e.g., alkyl, m, n, etc., when it occurs more than once in any structure, is intended to be independent of its definition elsewhere in the same structure.

The phrase “protecting group” as used herein means substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T.W.; Wuts, P.G.M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

Compounds of the present invention can exist as stereoisomers wherein asymmetric or chiral centers are present. These compounds are designated by the symbols “R” or “S,” depending on the configuration of substituents around the chiral carbon atom. The present invention contemplates various stereoisomers and mixtures thereof. Stereoisomers include enantiomers and diastereomers, and mixtures of enantiomers or diastereomers are designated (.+−.). Individual stereoisomers of compounds of the present invention can be prepared synthetically from commercially available starting materials which contain asymmetric or chiral centers or by preparation of racemic mixtures followed by resolution well-known to those of ordinary skill in the art. These methods of resolution include (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns.

The compounds of the present invention may also exist as geometric isomers. The present invention contemplates the various geometric isomers and mixtures thereof resulting from the arrangement of substituents around a carbon-carbon double bond or arrangement of substituents around a carbocyclic ring. Substituents around a carbon-carbon double bond are designated as being in the Z or E configuration wherein the term “Z” represents substituents on the same side of the carbon-carbon double bond and the term “E” represents substituents on opposite sides of the carbon-carbon double bond. The arrangement of substituents around a cycloalkane ring are designated as cis or trans wherein the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated cis/trans. Unless otherwise specified, a double bond is understood to include the Z or E configuration, or a mixture thereof. Similarly, unless otherwise specified, a cycloalkane ring is understood to encompass the cis or trans configuration, or a mixture thereof.

The compounds of the present invention may be used in the form of pharmaceutically-acceptable salts derived from inorganic or organic acids. By “pharmaceutically-acceptable salt” is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically-acceptable salts are well-known in the art. For example, S. M. Berge, et al. describe pharmaceutically-acceptable salts in detail in J. Pharmaceutical Sciences, 1977, 66: 1 et seq. The salts may be prepared in situ during the final isolation and purification of the compounds of the invention or separately by reacting a free base function with a suitable acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically-acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylaminonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Contemplated equivalents of the compounds described above include compounds which otherwise correspond thereto, and which have the same general properties thereof (e.g., functioning as inhibitors of leukotriene activity or histamine activity), wherein one or more simple variations of substituents are made which do not adversely affect the efficacy of the compound in inhibiting the above activities. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### Compounds of the Invention

In a further embodiment, the present invention relates to a compound of formula 1a:

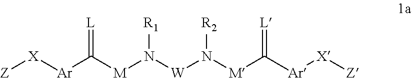

wherein, independently for each occurrence,
X and X′ represent independently O or S;
Z and Z′ each represent independently H, or optionally substituted alkyl, alkenyl, alkynyl, aryl, heteroaryl or acyl;
L and L′ each independently represent O, NR″, or S;
M and M′ independently represent a bond, or a bivalent alkyl or alkenyl chain.
W represents an optionally substituted bivalent alkyl, alkenyl, alkynyl, cycloalkyl, cylcolalkenyl, aryl or heteroaryl group, wherein the alkyl, alkenyl or alkynyl groups optionally contain one or more heteroatoms selected from O, S, or NR″′;
R₁ and R₂ represent H, or optionally substituted alkyl, alkenyl, alkynyl, aralkyl, cycloalkyl, aryl or heteroaryl; or R₁ and R₂ may be joined together to form an optionally substituted 4 to 8 membered heterocyclic ring, wherein the ring includes W and the nitrogens to which R₁ and R₂ are attached, provided that the heterocyclic ring is not unsubstituted piperazine, and provided that when R₁ and R₂ are both H, W is not bivalent n-propyl;
Ar and Ar′ independently represent optionally substituted aryl or heteroaryl;
R′, R″ and R″′ represent independently for each occurrence H, formyl, sulfonyl, or —(CH₂)ₘ—R₈₀, or optionally substituted alkyl, alkenyl, aryl, aralkyl, formyl or acyl;
R₈₀ represents independently for each occurrence optionally substituted aryl, cycloalkyl, cycloalkenyl, or heterocyclyl; and

or a pharmaceutically acceptable salt thereof.

In a further embodiment, the present invention relates to a compound of formula 1a, wherein X and X′ each represent O.

In a further embodiment, the present invention relates to a compound of formula 1a, wherein Z and Z′ each represent H. In another embodiment, Z and Z′ each represent optionally substituted alkyl, alkenyl, alkynyl, aryl, heteroaryl or acyl.

In a further embodiment, the present invention relates to a compound of formula 1a, wherein Ar and Ar′ independently represent optionally substituted phenyl or naphthyl. In another embodiment, Ar and Ar′ are optionally substituted with at least one member selected from the group consisting of halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, and sulfonamido. In another embodiment, Ar and Ar′ are optionally substituted with at least one halogen or alkoxy group. In another embodiment, the halogen is chloro. In another embodiment, Ar and Ar′ are independently unsubstituted aryl or naphthyl.

In another embodiment, the present invention relates to a compound of formula 1a, wherein M and M′ are each a bond. In another embodiment at least one of M and M′ is —CH═CH—. In another embodiment, one of M and M′ is a bond, and one of M and M′ is —CH═CH—.

In another embodiment, the present invention relates to a compound of formula 1a, wherein L and L′ independently represent O or NR′. In another embodiment, L and L′ independently represent O or NH. In another embodiment, L and L′ each represent O.

In a further embodiment, the present invention relates to compounds of formula 1a,′ wherein W represents a bivalent optionally substituted alkyl or cycloalkyl group, wherein the alkyl group optionally contains one or more heteroatoms selected from O, S, or NR″′. Examples of alkyl groups containing one or more heteroatoms include ethers, diethers, polyethers, sulfides, disulfides, dialkyl or trialkyl amines, diamines, and polyamines. In another embodiment, W represents a bivalent optionally substituted cyclohexyl group. In some embodiments, the cyclohexyl group is connected in the 1,2 position and has a cis configuration or a trans configuration.

In another embodiment, the present invention relates to compounds of formula 1a,′ wherein W represents —(CH₂)ₙ—, and wherein n is an integer ranging from 1 to 12, inclusive. In another embodiment, n is an integer from 2 to 6, inclusive. In another embodiment, n is 2, 4, 5, or 6.

In another embodiment, the present invention relates to compounds of formula 1a, wherein W represents —(CH₂)₂—S(CH₂)₂—, —(CH₂)₂—O—(CH₂)₂—O—(CH₂)₂—, —(CH₂)₃—N(CH₃)—(CH₂)₃—, —CH₂CH═CHCH₂—, —CH₂C(CH₃)₂CH₂—.

In another embodiment, the present invention relates to compounds of formula 1a,′ wherein R₁ and R₂ independently represent H, or optionally substituted alkyl; or R₁ and R₂ may be joined together to form an optionally substituted 4 to 8 membered heterocyclic ring, wherein the ring includes W and the nitrogens to which R₁ and R₂ are attached. For example, the heterocyclic ring comprises at least two nitrogen atoms, such as optionally substituted homopiperazine. In some embodiments, R₁ and R₂ independently represent H, methyl, ethyl, or propyl.

In another embodiment, the present invention relates to compounds of formula 1a,′ wherein X and X′ are each O; Z and Z′ are each H; L and L′ are each O; M and M′ are each a bond; and Ar and Ar′ are each phenyl or naphthyl;

In another embodiment, the present invention relates to compounds of formula 1b,

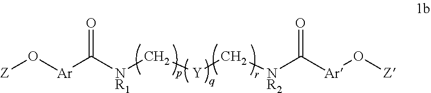

wherein, independently for each occurrence:

Y represents CH₂ or NR′;

R₁ and R₂ independently represent H, or optionally substituted alkyl or aryl, or R₁ and R₂ are joined together to form a optionally substituted 4 to 8 membered heterocyclic ring, wherein the ring includes —(CH₂)ₚ(Y)_{q}(CH₂)ᵣ— and the nitrogens to which R₁ and R₂ are attached; provided that the heterocyclic ring is not unsubstituted piperazine, and provided that when R₁ and R₂ are both H, W is not bivalent n-propyl;

p and r are 1, 2, or 3;

q is 0 or 1; and

Ar and Ar′ represent optionally substituted phenyl or substituted naphthyl;

Z and Z′ represent independently H or optionally substituted alkyl, alkenyl, alkynyl, aryl, heteroaryl or acyl;

or a pharmaceutically acceptable salt thereof.

In some embodiments, the present invention relates to compounds of formula 1b, wherein R₁ and R₂ independently represent H or alkyl.

In some embodiments, the present invention relates to compounds of formula 1b, wherein p and r are each 3.

In some embodiments, the present invention relates to compounds of formula 1b, wherein q is 1 and Y is NMe.

In some embodiments, the present invention relates to compounds of formula 1b, wherein Ar and Ar′ are each optionally substituted with at least one member selected from the group consisting of halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, and sulfonamido.

In another embodiment, the compound is selected from the group consisting of:

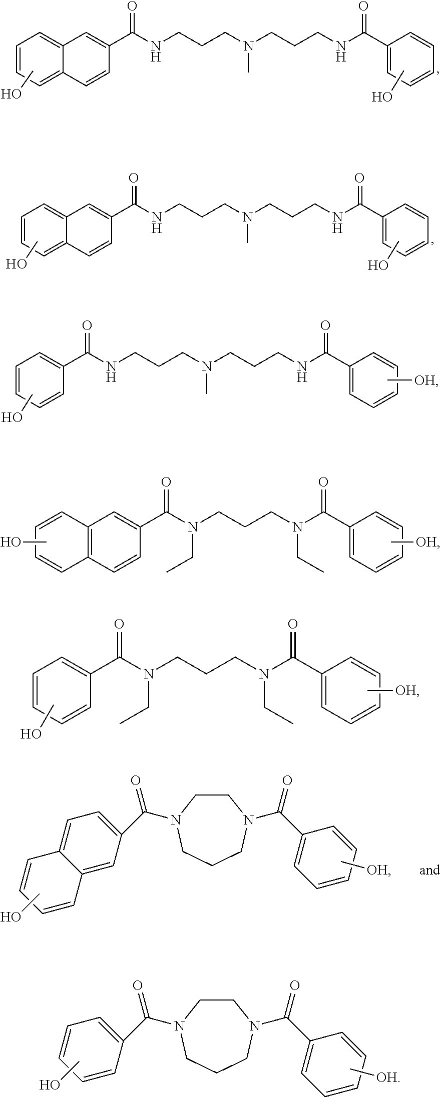

In another embodiment, the compound is:

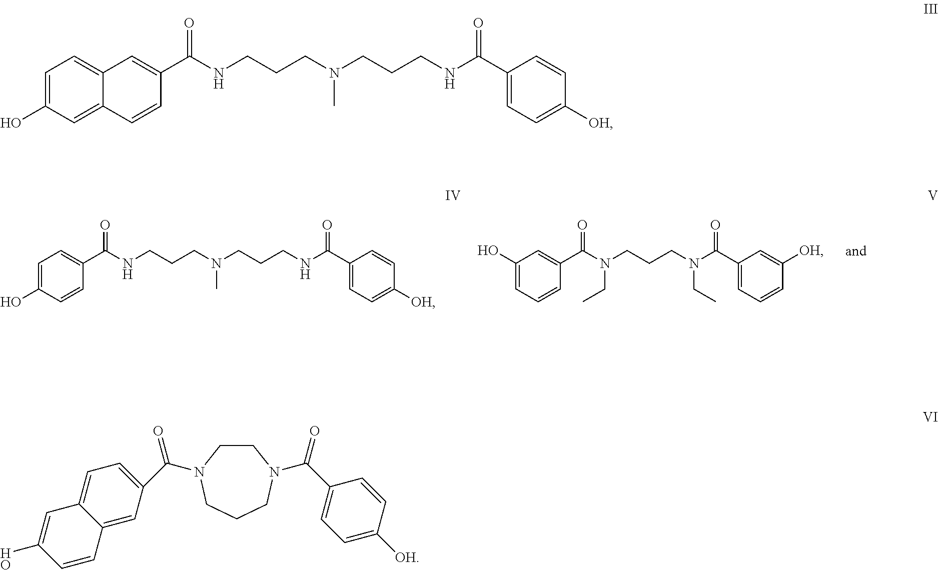

In certain embodiments, the present invention relates to a combinatorial library consisting of a plurality of compounds of the present invention.

In certain embodiments, the present invention relates to a method of inhibiting the adhesion to a surface by a bacterium, fungus, virion, freshwater invertebrate, or marine invertebrate, comprising the step of treating a surface with an effective amount of a compound of the present invention. In certain embodiments, said surface is a portion of an exterior surface of an aquatic vessel. In certain embodiments, said surface is a portion of an exterior surface of an off-shore platform. In certain embodiments, said surface is a portion of an exterior surface of a harbor infrastructure. In certain embodiments, said surface is a portion of an exterior or interior surface of a conduit for water or an aqueous solution. In certain embodiments, said surface is a portion of an exterior surface of a cable. In certain embodiments, said surface is a portion of an exterior or interior surface of a laboratory apparatus. In certain embodiments, said surface is a portion of an animal cell membrane. In certain embodiments, said surface is a portion of a mammalian cell membrane. In certain embodiments, said surface is a portion of a human cell membrane. In certain embodiments, said surface is a portion of an exterior surface of a plant. In certain embodiments, said surface is a portion of an exterior surface of a plant component. In certain embodiments, said surface is a portion of the cell membrane of a fungus. In certain embodiments, said surface is a portion of the cell wall of a fungus. In certain embodiments, said surface is a portion of the cell membrane of a bacterium. In certain embodiments, said surface is a portion of the cell wall of a bacterium. In certain embodiments, said surface is a portion of a virion protein coat. In certain embodiments, said surface is a portion of a virion envelope. In certain embodiments, said surface is a portion of an exterior surface of an implantable medical device. In certain embodiments, said surface is a portion of an exterior surface of an insertable medical device. In certain embodiments, said surface is a portion of a food processing surface.

In certain embodiments, the present invention relates to a method of enhancing the adhesion to a surface by a bacterium, fungus, virion, freshwater invertebrate, or marine invertebrate, comprising the step of treating a surface with an effective amount of a compound of the present invention. In certain embodiments, said surface is a portion of an exterior surface of an aquatic vessel. In certain embodiments, said surface is a portion of an exterior surface of an off-shore platform. In certain embodiments, said surface is a portion of an exterior surface of a harbor infrastructure. In certain embodiments, said surface is a portion of an exterior or interior surface of a conduit for water or an aqueous solution. In certain embodiments, said surface is a portion of an exterior surface of a cable. In certain embodiments, said surface is a portion of an exterior or interior surface of a laboratory apparatus. In certain embodiments, said surface is a portion of an animal cell membrane. In certain embodiments, said surface is a portion of a mammalian cell membrane. In certain embodiments, said surface is a portion of a human cell membrane. In certain embodiments, said surface is a portion of an exterior surface of a plant. In certain embodiments, said surface is a portion of an exterior surface of a plant component. In certain embodiments, said surface is a portion of the cell membrane of a fungus. In certain embodiments, said surface is a portion of the cell wall of a fungus. In certain embodiments, said surface is a portion of the cell membrane of a bacterium. In certain embodiments, said surface is a portion of the cell wall of a bacterium. In certain embodiments, said surface is a portion of a virion protein coat. In certain embodiments, said surface is a portion of a virion envelope. In certain embodiments, said surface is a portion of an exterior surface of an implantable medical device. In certain embodiments, said surface is a portion of an exterior surface of an insertable medical device. In certain embodiments, said surface is a portion of a food processing surface.

### Synthesis of Compounds

Compound synthesis involves serial additions of carbonyl aryl groups to the linker moiety as depicted in Scheme 1.

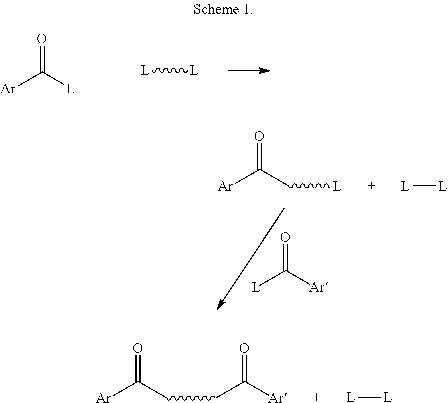

where Ar and Ar′ represent aryl groups and Ls independently represent reactive groups. For example, the L's may be nucleophiles, leaving groups, or electrophiles, or the L's may be reactive groups that undergoing a coupling reaction in the presence of an organometallic catalyst. Such reactions are well known to those of ordinary skill in the art.

In one embodiment, compounds of the present invention are prepared by sequentially reacting a phenyl carbonyl group and a naphthalenyl carbonyl group with a bivalent linker comprising terminal amine groups as depicted in Scheme 2.

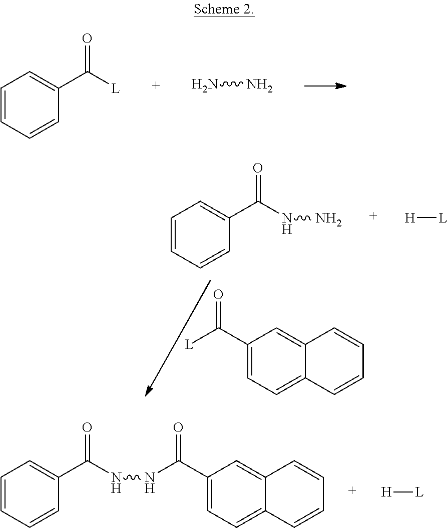

wherein Ls are leaving groups and wherein the aryl rings may be optionally substituted.

It is apparent by this general synthetic scheme that both symmetric and asymmetric compounds may be prepared by reacting the correct molar amounts. Scheme 3 depicts the synthesis of compound 1 which is an asymmetric compound wherein the aryl rings are substituted with hydroxy groups.

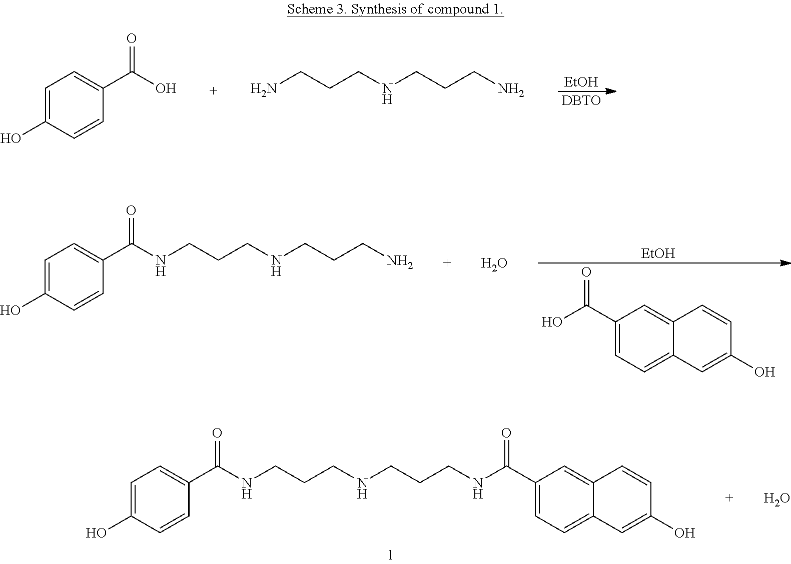

where DBTO stands for dibutyltin oxide.

### Results

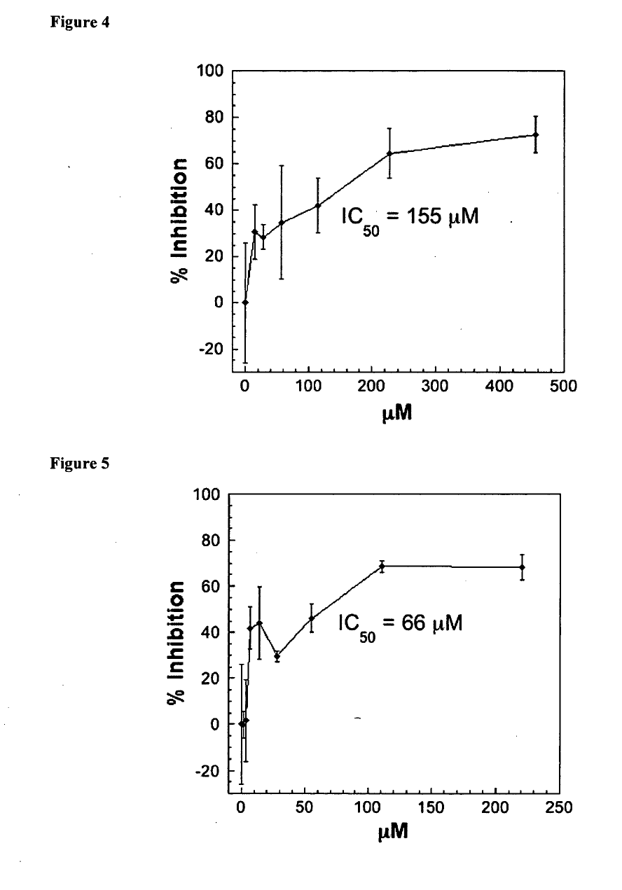

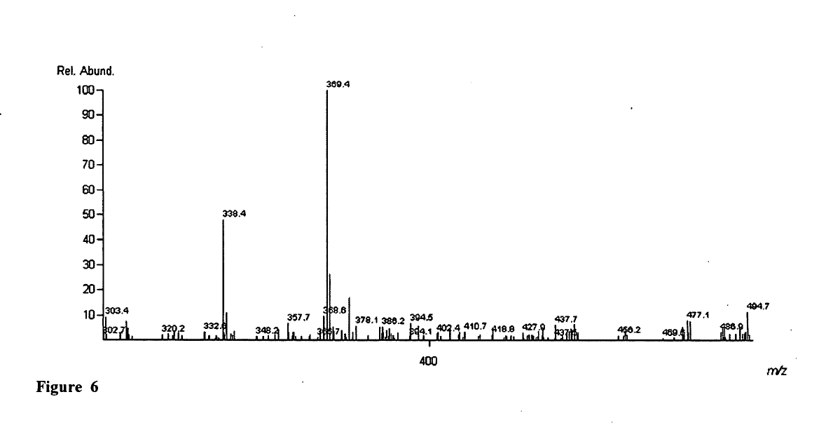

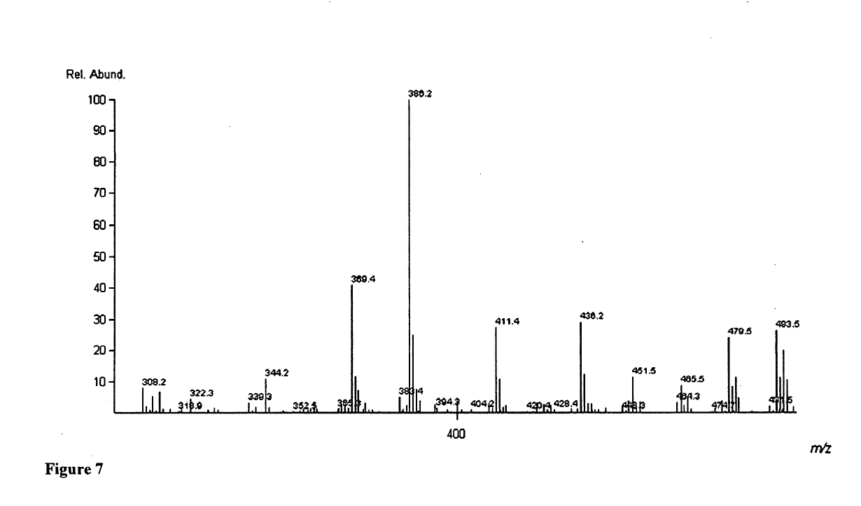

Compounds of the present invention were tested for effectiveness as anti-adhesions against microbial agents including viruses (Human Influenza A and Dengue 2) and bacteria (Staphylococcus aureus). IC₅₀ (μM) values measured from graphs of % inhibition versus concentration for compounds of the present invention against Influenza A (see FIG. 4 for compound 1 and FIG. 5 for compound 4) and Dengue 2 are given in Table 1.

TABLE 1
Anti-infection (adhesion) of viruses.

IC₅₀ (μM)

| Compound | Influenza A | Dengue 2 |
|---|---|---|
| I | 66 | N/A |
| IV | 4,078 | N/A |
| III | 155 | — |
| VI | — | 114 |
| V | N/A | 2,328 |
| Zosteric Acid | 858 | 1,189 |

IC₅₀ (μM) values for compounds of the present invention against for MRSA Staphylococcus aureus are given in Table 2.

TABLE 2
Summary of anti-adhesion data for MRSA Staphylococcus aureus for
compounds of the present invention.

| Compound | IC₅₀ (μM) | IC_{MAX} (μM) |
|---|---|---|
| Zosteric Acid | 24.80 | IC₁₀₀ - 90 |
| IV | 1.50 | IC₉₀ - 10 |
| V (salt) | 1.13 | ND |
| V | 1.05 | ND |
| I (salt) | 765 | IC₉₅ - 1000 |
| I | 82 | IC₇₀ - 120 |
| III | 7.1 | IC₁₀₀ - 100 |
| VI | 2.5 | IC₁₀₀ - 10 |

Table 3 summarizes the applications (as measured by IC₅₀ (% wt/vol) values) of compounds of the present invention towards gram negative bacteria (S. aureus), cystic fibrosis (P. aerug) and yeast (C. acut.).

TABLE 3
Effectiveness of compounds of the present invention
towards different applications.

| | | IC₅₀ | | IC₅₀ | | IC₅₀ |
|---|---|---|---|---|---|---|
| Compound | Pathogen | (% wt/vol) | Pathogen | (% wt/vol) | Pathogen | (% wt/vol) |
| III | S. aure. | <0.017 | P. aerug. | 0.001 | C. acut. | <0.005 |
| I | S. aure. | NM | P. aerug. | 0.001 | C. acut. | 0.004 |
| VI | S. aure. | <0.017 | P. aerug. | <0.005 | C. acut. | <0.005 |

### Applications

Disclosed herein are compounds, and combinatorial libraries thereof, for use on surfaces susceptible to adhesion by various biologics. Such “antifouling” substances may be employed to prevent damage by biologics to such surfaces, prevent formation of biofilms on such surfaces, prevent infection by biologics of such surfaces, suppress thrombogenic properties of such surfaces, and other uses readily apparent to those skilled in the appropriate arts. Examples of biologics include, but are not limited to, bacteria, fungi, viruses, protozoans, and marine invertebrates.

In certain embodiments, compounds of the invention may prevent fouling by biologics of surfaces in prolonged contact with aqueous environments. Examples of such surfaces include, but are not limited to, hulls of ships immersed in sea- or fresh water, laboratory equipment immersed in water-filled incubators, and water conduits, such as pipes or vaporization apparatus immersed in water reservoirs of air humidifiers. In certain embodiments, these compounds may prevent fouling by algae, barnacles, barnacle larvae, diatoms, mussels, and other marine- and fresh-water organisms known to one of ordinary skill in the art.

In certain embodiments, these compounds affect the agglutination of bacterial and mammalian cells. The effects of these compounds on cell agglutination may involve the blocking of certain cell surface receptors and the activation of others, such as those involved in the attachment to extracellular surfaces and which thereby mediate fouling. Thus, these compounds may possess many of the activities of naturally-occurring proteins and glycoproteins which bind to sites on the surface of a cell and thereby affect cell/cell interactions.

The instant claimed compounds interfere with the attachment of organisms to surfaces, thereby having broad applicability in effectively inhibiting the attachment of a variety of organisms. In addition, the compounds are relatively safe for wide-spread environmental use, as they naturally degrade into carbon dioxide and water, or simple organic acids.

In certain embodiments, compounds have a relatively short half-life after release, rendering them particularly well-suited for widespread environmental use. In certain embodiments, compounds can be readily synthesized.

In certain embodiments, compounds of the instant invention may prevent infection of plants and plant components by plant pathogens. “Plant component” refers to a portion or part of a plant. Examples include: seeds, roots, stems, vascular systems, fruits (further including pip fruits (e.g. apples, pears, quinces)), citrus fruits (oranges, lemons, limes, grapefruits, mandarins, nectarines), stone fruits (peaches apricots, plums, cherries, avocados, grapes), berries (strawberries, blueberries, raspberries, blackberries)), leaves, grains and vegetables.

A “plant pathogen” refers to an organism (bacteria, virus, protist, algae or fungi) that infects plants of plant components. Examples include molds, fungi and rot that typical use spores to infect plants or plant components (e.g. fruits, vegetables, grains, stems, roots). Spores must recognize the host, attach, germinate, penetrate host tissues, and proliferate hyphae that will allow the fungus access to nutrients for growth and reproduction. Examples include: Botrytis sp. (B. cinerea), Penicillium sp. (P. expansum, P. italicum, P. digitalum), Rhizopus sp. (R. sulonifer, R. nigricans), Alternaria sp. (A. alternate, A. solani), Diploidia sp. (D. natalenses), Monilinia sp. (M. fructicola), Pseudomonas sp. (P. cepacia), Xanthomonas sp., Erwinia sp., Corynebacterium sp., Cladosporium sp. (C. fulva), Phytophtora sp. (P. infestans), Colletotricum sp. (C. coccoides C. fragariae, C. gloesporioides), Fusarium sp. (F. lycopersici), Verticillium sp. (V. alboatrum, V. dahliae), Unicula sp. (U. necator), Plasmopara sp. (P. viticola), Guignardia sp. (G. bidwellii), Cercospora sp. (C. arachidicola), Scelrotinia sp. (S. scerotiorum), Puccinia sp. (P. arachidis), Aspergillus sp. (A. flavus), Venturia sp. (V. inaequalis) Podosphaera sp. (P. leucotricha), Pythiun sp., Sphaerotheca sp. (S. macularis), and Bacillus sp. (B. subitlis).

In certain embodiments, compounds of the invention may provide one or more plant and animal lectin-like activities. Lectins bind to cell surface proteoglycans, which function in the attachment of pathogens such as viruses and bacteria. Accordingly the lectin-like activities of compounds of the invention are useful in treating and preventing infections and other receptor-mediated diseases and conditions.

The ability of the compounds to bind to certain cell surface sites is useful for agonizing or antagonizing certain cell surface interactions which are otherwise affected by animal or plant lectin proteins. The extracellular polysaccharides produced by fouling organisms are often highly sulfated, and these sulfate esters play an important role in polymerization (e.g. glue/gel formation).

In certain embodiments, compounds of the invention may prevent infection of humans by human pathogens, including but not limited to bacteria, viruses, protozoans, parasites, and fungi. Infection may be prevented by inhibiting adhesion of such pathogens to cell membranes and tissue surfaces in humans. In certain embodiments, compounds of the invention may similarly prevent infection of mammals and other animals by biologics.

In certain embodiments, compounds of the invention may inhibit attachment of parasitic fungi spores to plants, as well as hyphal production from previously attached spores. Even after prolonged exposure, the presence of the compounds of the invention on the plants did not result in any toxic or growth inhibitory effect.

By blocking spore attachment, an initial step in the infection process, the compounds of the invention may provide a highly effective antifungal and antibacterial agent. In addition, the compounds of the present invention are also capable of interfering with the attachment, germination, penetration by fungal plant pathogens. Further, because essentially all fungal plant pathogens use spores to recognize the host plant, attach, germinate, penetrate the host plant tissue and proliferate hyphae that allows the fungus access to the plant's nutrients for growth and reproduction, the compounds are broad-based antifungal agents. In addition a series of investigations on several species of bacteria, microalgae, macroalgal spores and invertebrates has confirmed that the inhibitory mode-of-action is through a non-toxic means (Zimmerman et al., (1995) U.S. Pat. No. 5,384,176; Zimmerman et al., (1997) U.S. Pat. No. 5,607,741; Todd et al., Phytochemistry 34: 401-404 (1993); Sundberg et al., Naval Research Reviews (1997) 4: 51-59).

The compounds of the invention can also be used to inhibit or to destroy the microorganisms occurring on plants or on parts of plants (the fruit, blossom, leaves, stems, tubers or roots) of various crops of useful plants, while at the same time parts of plants that grow later are also protected against such microorganisms. They can also be used as dressings in the treatment of plant propagation material, especially seed (fruit, tubers, grains) and plant cuttings (for example rice), to provide protection against fungal infections and against phytopathogenic fungi occurring in the soil. The compound mixtures according to the invention are distinguished by the fact that they are especially well tolerated by plants and are environmentally friendly.

Target crops to be protected within the scope of the present invention comprise, for example, the following species of plants: cereals (wheat, barley, rye, oats, rice, sorghum and related species); beets (sugar beet and fodder beet); pomes, stone fruit and soft fruit (apples, pears, plums, peaches, almonds, cherries, strawberries, raspberries and blackberries); leguminous plants (beans, lentils, peas and soybeans); oil plants (rape, mustard, poppy, olives, sunflowers, coconut, castor oil plants, cocoa beans and groundnuts); cucumber plants (marrows, cucumber and melons); fiber plants: (cotton, flax, hemp and jute); citrus fruit (oranges, lemons, grapefruit and mandarins); vegetables (spinach, lettuce, asparagus, cabbages, carrots, onions, tomatoes, potatoes and paprika); lauraceae (avocados, cinnamon and camphor); or plants such as maize, tobacco, nuts, coffee, sugar cane, tea, vines, hops, bananas and natural rubber plants, as well as ornamental plants (flowers, shrubs, broad-leaved trees and evergreens, such as conifers).

One of skill in the art will recognize that the composition of the invention can be varied as required to optimize the overall chemical properties of the particular compound for specific uses, while retaining the antifouling (AF) activity. For example, the length of an alkyl chain can be extended or shortened to control the rate of dissolution of the compound from a structure or a coating. Alternatively, additional functional groups can be added to the alkyl chain to further vary the chemical nature of the molecule.

Implantable medical devices, using artificial materials alone or in combination with naturally-derived materials, can be treated with compounds either by surface coating or by incorporation. Metals may be suitably treated with surface coats while retaining their biological properties. In certain embodiments of the present invention, metals may be treated with paints or with adherent layers of polymers or ceramics that incorporate the compounds of the invention. Certain embodiments treated in this manner may be suitable for orthopedic applications, for example, pins, screws, plates or parts of artificial joints. Methods for surface treatment of metals for biological use are well-known in the relevant arts. Other materials besides metals can be treated with surface coats of compounds according to the present invention as the medical application requires.

Implantable devices may comprise materials suitable for the incorporation of the instant claimed compounds. Embodiments whose components incorporate compounds of the invention can include polymers, ceramics and other substances. Materials fabricated from artificial materials can also be destined for resorption when they are placed in the body. Such materials can be called bioabsorbable. As an example, polyglycolic acid polymers can be used to fabricate sutures and orthopedic devices. Those of ordinary skill in these arts will be familiar with techniques for incorporating agents into the polymers used to shape formed articles for medical applications. AF agents can also be incorporated into glues, cements or adhesives, or in other materials used to fix structures within the body or to adhere implants to a body structure. Examples include polymethylmethacrylate and its related compounds, used for the affixation of orthopedic and dental prostheses within the body. The presence of the compounds of the instant invention can decrease biofilm formation in those structures in contact with the glue, cement, or adhesive. Alternatively, a compound of the invention can coat or can permeate the formed article. In these compositions, the formed article allows diffusion of the compound, or functional portion thereof, into the surrounding environment, thereby preventing fouling of the appliance itself. Microcapsules bearing compounds can also be imbedded in the material. Materials incorporating compounds are adaptable to the manufacture of a wide range of medical devices, some of which are disclosed below. Other examples will be readily apparent to those practitioners of ordinary skill in the art.

In one embodiment, compounds of the invention can be applied to or incorporated in certain medical devices that are intended to be left in position permanently to replace or restore vital functions. As one example, ventriculoatrial or ventriculoperitoneal shunts are devised to prevent cerebrospinal fluid from collecting in the brain of patients whose normal drainage channels are impaired. As long as the shunt functions, fluid is prevented from accumulating in the brain and normal brain function can continue. If the shunt ceases to function, fluid accumulates and compresses the brain, with potentially life-threatening effect. If the shunt becomes infected, it causes an infection to enter the central portions of the brain, another life-threatening complication. These shunts commonly include a silicone elastomer or another polymer as part of their fabrication. Silicones are understood to be especially suited for combination with compounds according to the present invention.

Another shunt that has life-saving import is a dialysis shunt, a piece of polymeric tubing connecting an artery and a vein in the forearm to provide the kidney failure patient a means by which the dialysis equipment can cleanse the bloodstream. Even though this is a high-flow conduit, it is susceptible to the formation of biofilms and subsequent infection. If a shunt becomes infected, it requires removal and replacement. Since dialysis may be a lifelong process, and since there are a limited number of sites where shunts can be applied, it is desirable to avoid having to remove one through infectious complications. Imbedding or otherwise contacting the compounds of the invention with the shunt material can have this desired effect.

Heart valves comprising artificial material are understood to be vulnerable to the dangerous complication of prosthetic valve endocarditis. Once established, it carries a mortality rate as high as 70%. Biofilms are integrally involved in the development of this condition. At present, the only treatment for established contamination is high-dose antibiotic therapy and surgical removal of the device. The contaminated valve must be immediately replaced, since the heart cannot function without it. Because the new valve is being inserted in a recently contaminated area, it is common for prosthetic valve endocarditis to affect the replacement valve as well. Artificial heart valves comprised of the compounds of the invention may reduce the incidence of primary and recurrent prosthetic valve endocarditis. Compounds of the invention can be applied to the synthetic portions or the naturally-derived portions of heart valves.

Pacemakers and artificial implantable defibrillators commonly comprise metallic parts in combination with other synthetic materials. These devices, which may be coated with a polymeric substance such as silicone are typically implanted in subcutaneous or intramuscular locations with wires or other electrical devices extending intrathoracically or intravascularly. If the device becomes colonized with microorganisms and infected, it must be removed. A new device can be replaced in a different location, although there are a finite number of appropriate implantation sites on the body. Devices comprising the compounds of the invention may inhibit contamination and infection, or substantially reduce the risk thereof.

Devices implanted into the body either temporarily or permanently to pump pharmacological agents into the body can comprise metallic parts in combination with other synthetic materials. Such devices, termed infusion pumps, can be entirely implanted or can be partially implanted. The device may be partially or entirely covered with a polymeric substance, and may comprise other polymers used as conduits or tubes. Incorporating AF agents according to the present invention into the coating materials imposed upon these devices or into the materials used for the devices themselves, their conduits or their tubing may inhibit their contamination and infection.

Equally lifesaving are the various vascular grafting prostheses and stents intended to bypass blocked arteries or substitute for damaged arteries. Vascular grafting prostheses, made of Teflon, Dacron, Gore-tex®, expanded polytetrafluoroethylene (e-PTFE), and related materials, are available for use on any major blood vessel in the body. Commonly, for example, vascular grafting prostheses are used to bypass vessels in the leg and are used to substitute for a damaged aorta. They are put in place by being sewn into the end or the side of a normal blood vessel upstream and downstream of the area to be bypassed or replaced, so that blood flows from a normal area into the vascular grafting prosthesis to be delivered to other normal blood vessels. Stents comprising metallic frames covered with vascular grafting prosthesis fabric are also available for endovascular application, to repair damaged blood vessels.

When a vascular grafting prosthesis becomes infected, it can spread infection through the entire bloodstream. Furthermore, the infection can weaken the attachment of the vascular grafting prosthesis to the normal blood vessel upstream or downstream, so that blood can leak out of it. If the attachment ruptures, there can be life-threatening hemorrhage. When a vascular grafting prosthesis becomes infected, it needs to be removed. It is especially dangerous to put another vascular grafting prosthesis in the same spot because of the risk of another infection, but there are often few other options. Vascular grafting prostheses comprising compounds of the invention can resist infections, thereby avoiding these devastating complications.

Vascular grafting prostheses of small caliber are particularly prone to clotting. A vascular grafting prosthesis comprising a compound of the invention may not only prevent biofilm formation, but also inhibit clotting as described above, allowing a smaller diameter vascular grafting prosthesis to be more reliable. A common site for clotting is the junction point between the vascular grafting prosthesis and the normal vessel, called the anastomosis. Even if an artificial vascular grafting prosthesis is not used, anywhere that two vessels are joined or anywhere there is a suture line that penetrates a natural blood vessel, there is a potential for clotting to take place. A clot in a vessel can occlude the vessel entirely or only partially; in the latter case, blood clots can be swept downstream, damaging local tissues. Using suture comprised of the compounds of the invention may inhibit clotting at these various suture lines. The smaller the vessel, the more likely that a clot forming within it will lead to a total occlusion. This can have disastrous results: if the main vessel feeding a tissue or an organ becomes totally occluded, that structure loses its blood supply and can die. Microsurgery provides dramatic examples of the damage that can occur with anastomotic clotting. In microsurgery, typically only a single tiny vessel is feeding an entire tissue structure like a finger or a muscle. If the vessel clots off, the tissue structure can quickly die. Microsurgery typically involves vessels only one to four millimeters in diameter. It is understood that the sutures penetrating the vessel at the anastomosis are likely sites for clots to form. Microsurgical sutures comprising a compound of the invention would result in localized administration of an anticoagulant at the site most likely to be damaged by clotting.

Suture material used to anchor vascular grafting prostheses to normal blood vessels or to sew vessels or other structures together can also harbor infections. Sutures used for these purposes are commonly made of prolene, nylon or other monofilamentous nonabsorbable materials. An infection that begins at a suture line can extend to involve the vascular grafting prosthesis. Suture materials comprising a compound of the invention would have increased resistance to infection.

A suture comprising a compound of the invention would be useful in other areas besides the vasculature. Wound infections at surgical incisions may arise from microorganisms that lodge in suture materials placed at various levels to close the incision. General surgery uses both nonabsorbable and absorbable sutures. Materials for nonabsorbable sutures include prolene and nylon. Absorbable sutures include materials like treated catgut and polyglycolic acid. Absorbable sutures retain tensile strength for periods of time from days to months and are gradually resorbed by the body. Fabricating an absorbable or a nonabsorbable suture comprising a compound of the invention and which retains the handling and tensile characteristics of the material is within the skill of artisans in the field.

Medical prostheses comprising compounds of the invention would be expected to have reduced contamination and subsequent local infection, thereby obviating or reducing the need to remove the implant with the attendant destruction of local tissues. Destruction of local tissues, especially bones and ligaments, can make the tissue bed less hospitable for supporting replacement prosthesis. Furthermore, the presence of contaminating microorganisms in surrounding tissues makes recontamination of the replacement prosthesis easily possible. The effects of repeated contamination and infection of structural prosthetics is significant: major reconstructive surgery may be required to rehabilitate the area in the absence of the prosthesis, potentially including free bone transfers or joint fusions. Furthermore, there is no guarantee that these secondary reconstructive efforts will not meet with infectious complications as well. Major disability, with possible extremity amputation, is the outcome from contamination and infection of a structural prosthesis.

Tissue expanders are sacs made of silicone elastomers adapted for gradual filling with a saline solution, whereby the filling process stretches the overlying tissues to generate an increased area of tissue that can be used for other reconstructive applications. Tissue expanders can be used, for example, to expand chest wall skin and muscle after mastectomy as a step towards breast reconstruction. Tissue expanders can also be used in reconstructing areas of significant skin loss in burn victims. A tissue expander is usually intended for temporary use: once the overlying tissues are adequately expanded, they are stretched to cover their intended defect. If a tissue expander is removed before the expanded tissues are transposed, though, all the expansion gained over time is lost and the tissues return nearly to their pre-expansion state. The most common reason for premature tissue expander removal is infection. These devices are subjected to repeated inflations of saline solution, introduced percutaneously into remote filling devices that communicate with the expander itself. Bacterial contamination of the device is thought to occur usually from the percutaneous inflation process. Once contamination is established and a biofilm forms, local infection is likely. Expander removal, with the annulment of the reconstructive effort, is needed to control the infection. A delay of a number of months is usually recommended before a new tissue expander can be inserted in the affected area. The silicone elastomer used for these devices is especially suitable for integrating with sulfate ester AF agents. Use of these agents in the manufacture of these articles may reduce the incidence of bacterial contamination, biofilm development and subsequent local infection.

Insertable devices include those objects made from synthetic materials applied to the body or partially inserted into the body through a natural or an artificial site of entry. Examples of articles applied to the body include contact lenses and stoma appliances. An artificial larynx is understood to be an insertable device in that it exists in the airway, partially exposed to the environment and partially affixed to the surrounding tissues. An endotracheal or tracheal tube, a gastrostomy tube or a catheter are examples of insertable devices partially existing within the body and partially exposed to the external environment. The endotracheal tube is passed through an existing natural orifice. The tracheal tube is passed through an artificially created orifice. Under any of these circumstances, the formation of biofilm on the device permits the ingress of microorganisms along the device from a more external anatomic area to a more internal anatomic area. The ascent of microorganisms to the more internal anatomic area commonly causes local and systemic infections.

As an example, biofilm formation on soft contact lenses is understood to be a risk factor for contact-lens associated corneal infection. The eye itself is vulnerable to infections due to biofilm production. Incorporating an antifouling agent in the contact lens itself and in the contact lens case can reduce the formation of biofilms, thereby reducing risk of infection. Sulfate ester AF agents can also be incorporated in ophthalmic preparations that are periodically instilled in the eye.

As another example, biofilms are understood to be responsible for infections originating in tympanostomy tubes and in artificial larynxes. Biofilms further reside in tracheostomy tubes and in endotracheal tubes, permitting the incursion of pathogenic bacteria into the relatively sterile distal airways of the lung. These devices are adaptable to the incorporation or the topical application of sulfate ester AF agents to reduce biofilm formation and subsequent infectious complications.

As another example, a wide range of vascular catheters are fabricated for vascular access. Temporary intravenous catheters are placed distally, while central venous catheters are placed in the more proximal large veins. Catheter systems can include those installed percutaneously whose hubs are external to the body, and those whose access ports are buried beneath the skin. Examples of long-term central venous catheters include Hickman catheters and Port-a-caths. Catheters permit the infusion of fluids, nutrients and medications; they further can permit the withdrawal of blood for diagnostic studies or the transfusion of blood or blood products. They are prone to biofilm formation, increasingly so as they reside longer within a particular vein. Biofilm formation in a vascular access device can lead to the development of a blood-borne infection as planktonic organisms disseminate from the biofilm into the surrounding bloodstream. Further, biofilm formation can contribute to the occlusion of the device itself, rendering it non-functional. If the catheter is infected, or if the obstruction within it cannot be cleared, the catheter must be removed. Commonly, patients with these devices are afflicted with serious medical conditions. These patients are thus poorly able to tolerate the removal and replacement of the device. Furthermore, there are only a limited number of vascular access sites. A patient with repeated catheter placements can run out of locations where a new catheter can be easily and safely placed. Incorporation of sulfate ester AF agents within catheter materials or application of these agents to catheter materials can reduce fouling and biofilm formation, thereby contributing to prolonged patency of the devices and minimizing the risk of infectious complications.

As another example, a biliary drainage tube is used to drain bile from the biliary tree to the body's exterior if the normal biliary system is blocked or is recovering from a surgical manipulation. Drainage tubes can be made of plastics or other polymers. A biliary stent, commonly fabricated of a plastic material, can be inserted within a channel of the biliary tree to keep the duct open so that bile can pass through it. Biliary sludge which forms as a result of bacterial adherence and biofilm formation in the biliary system is a recognized cause of blockage of biliary stents. Pancreatic stents, placed to hold the pancreatic ducts open or to drain a pseudocyst of the pancreas, can also become blocked with sludge. Biofilms are furthermore implicated in the ascent of infections into the biliary tree along a biliary drainage tube. Ascending infections in the biliary tree can result in the dangerous infectious condition called cholangitis. Incorporation of compounds of the invention in the materials used to form biliary drainage tubes and biliary stents can reduce the formation of biofilms, thereby decreasing risk of occlusions and infections.

As another example, a peritoneal dialysis catheter is used to remove bodily wastes in patients with renal failure by using fluids instilled into and then removed from the peritoneal cavity. This form of dialysis is an alternative to hemodialysis for certain renal failure patients. Biofilm formation on the surfaces of the peritoneal dialysis catheter can contribute to blockage and infection. An infection entering the peritoneal cavity is termed a peritonitis, an especially dangerous type of infection. Peritoneal dialysis catheters, generally made of polymeric materials like polyethylene, can be coated with or impregnated with sulfate ester AF agents to reduce the formation of biofilms.

As yet another example, a wide range of urological catheters exist to provide drainage of the urinary system. These catheters can either enter the natural orifice of the urethra to drain the bladder, or they can be adapted for penetration of the urinary system through an iatrogenically created insertion site. Nephrostomy tubes and suprapubic tubes represent examples of the latter. Catheters can be positioned in the ureters on a semipermanent basis to hold the ureter open; such a catheter is called a ureteral stent. Urological catheters can be made from a variety of polymeric products. Latex and rubber tubes have been used, as have silicones. All catheters are susceptible to biofilm formation. This leads to the problem of ascending urinary tract infections, where the biofilm can spread proximally, carrying pathogenic organisms, or where the sessile organisms resident in the biofilm can propagate planktonic organisms that are capable of tissue and bloodstream invasion. Organisms in the urinary tract are commonly gram-negative bacteria capable of producing life-threatening bloodstream infections if they spread systemically. Infections wherein these organisms are restricted to the urinary tract can nonetheless be dangerous, accompanied by pain and high fever. Urinary tract infections can lead to kidney infections, called pyelonephritis, that can jeopardize the function of the kidney. Incorporating sulfate ester AF agents can inhibit biofilm formation and may reduce the likelihood of these infectious complications.

A further complication encountered in urological catheters is encrustation, a process by which inorganic compounds comprising calcium, magnesium and phosphorous are deposited within the catheter lumen, thereby blocking it. These inorganic compounds are understood to arise from the actions of certain bacteria resident in biofilms on catheter surfaces. Reducing biofilm formation by the action of sulfate ester AF agents may contribute to reducing encrustation and subsequent blockage of urological catheters.

Other catheter-like devices exist that can be treated with AF agents. For example, surgical drains, chest tubes, hemovacs and the like can be advantageously treated with materials to impair biofilm formation. Other examples of such devices will be familiar to ordinary practitioners in these arts.

Materials applied to the body can advantageously employ the AF compounds disclosed herein. Dressing materials can themselves incorporate the AF compounds, as in a film or sheet to be applied directly to a skin surface. Additionally, AF compounds of the instant invention can be incorporated in the glue or adhesive used to stick the dressing materials or appliance to the skin. Stoma adhesive or medical-grade glue may, for example, be formulated to include an AF agent appropriate to the particular medical setting. Stoma adhesive is used to adhere stoma bags and similar appliances to the skin without traumatizing the skin excessively. The presence of infectious organisms in these appliances and on the surrounding skin makes these devices particularly appropriate for coating with AF agents, or for incorporating AF agents therein. Other affixation devices can be similarly treated. Bandages, adhesive tapes and clear plastic adherent sheets are further examples where the incorporation of an AF agent in the glue or other adhesive used to affix the object, or incorporation of an AF agent as a component of the object itself, may be beneficial in reducing skin irritation and infection.

These above examples are offered to illustrate the multiplicity of applications of compounds of the invention in medical devices. Other examples will be readily envisioned by skilled artisans in these fields. The scope of the present invention is intended to encompass all those surfaces where the presence of fouling has adverse health-related consequences. The examples given above represent embodiments where the technologies of the present invention are understood to be applicable. Other embodiments will be apparent to practitioners of these and related arts. Embodiments of the present invention can be compatible for combination with currently employed antiseptic regimens to enhance their antimicrobial efficacy or cost-effective use. Selection of an appropriate vehicle for bearing a compound of the invention will be determined by the characteristics of the particular medical use. Other examples of applications in medical environments to promote antisepsis will be readily envisioned by those of ordinary skill in the relevant arts.

Antifouling coating compositions of the present invention can also contain a paint base such as rosin, silicone, vinyl, acrylic, and alkyd resin bases. They can also contain a pigment such as titanium dioxide, a thickener such as bentonite, fillers such as aluminum silicate and calcium silicate, and driers such as cobalt naphthenate and manganese naphthenate. They may also contain solvents or thinners such as mineral spirits, naphtha, benzene, toluene, methylethyl ketone, and the like.

Biological contamination on food processing surfaces is a potential source of bacterial contamination of foods and can result in decreased shelf life or transmission of disease. The possible presence of biological contamination on medically related devices, such as surgical and medical instruments, implants and prosthetics is also of serious concern. It would also be desirable to treat surfaces of a variety of other materials, e.g., upholstery fabrics, plastic food packaging films, and so forth, to make them resistant to the deposit and adhesion of bacteria and other biological materials.

Due to their ability to interfere with the attachment of organisms to surfaces, the compounds of the present invention have broad applicability in inhibiting a variety of organisms that contribute to the formation of biofilms or are otherwise involved with biofouling. Additionally, the compounds of the instant invention have utility as environmentally benign crop protection agents because they are capable of preventing attack of various fungi species upon seeds, seedlings, and mature crop plants, and bacteria.

Further, adhesion of pathologic biologics to the surface of host cells is often a required step in the pathogenesis of disease. Therefore, substances, such as the compounds of the instant invention, that interfere with the ability of pathologic microorganisms to adhere to the surface of host cells may have use in preventing initiation of cellular infection.

### Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or oral cavity; or (4) intravaginally or intravectally, for example, as a pessary, cream or foam; (5) sublingually; (6) ocularly; (7) transdermally; or (8) nasally.

The compounds according to the invention may be formulated for administration in any convenient way for use in human or veterinary medicine, by analogy with other pharmaceuticals.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

### Combinatorial Libraries

The subject methods readily lend themselves to the creation of combinatorial libraries of compounds for the screening of pharmaceutical, agrochemical or other biological or medical activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lerner et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, which is particularly suitable for discovering enzyme inhibitors, the beads can be dispersed on the surface of a permeable membrane, and the diversomers released from the beads by lysis of the bead linker. The diversomer from each bead will diffuse across the membrane to an assay zone, where it will interact with an enzyme assay. Detailed descriptions of a number of combinatorial methodologies are provided below.

### A) Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

### B) Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

### C) Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

### D) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

### E) Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-Sequenceable Tagging: Binary Encoding

An alternative form of encoding the test compound library employs a set of non-sequenceable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

Synthesis of compound 1. To a 1 liter 4-necked round bottomed flask, fitted with a distillation apparatus, was added 3,3′-diamino-N-methyldipropylamine (45.835 g, 0.316 mol), absolute ethanol (325 ml), ethyl-4-hydroxybenzoate (52.439 g, 0.316 mol), and dibutyltin oxide (0.235 g; 8.25 mmol). The reaction mixture was heated to 170° C. within 2 h under an inert nitrogen atmosphere, removing the ethanol via distillation. After 8 h at 170° C., the translucent yellow melt was allowed to cool to room temperature under nitrogen. Once at room temperature, absolute ethanol (300 ml) and ethyl-6-hydroxy-2-naphthoate (67.833 g; 0.314 mol) was added to the white solid residue in the reaction flask to afford an orange translucent liquid. The reaction mixture was heated to 170° C. within 2 h under an inert nitrogen atmosphere, removing the ethanol via distillation. After 8 h at 170° C., the dark orange melt was allowed to cool to room temperature under nitrogen. After successive washes with ethanol, ethyl acetate, and ether, compound 1 (N-(3-{[N-(3-propyl)-6-hydroxy-2-naphthamide]-methyl-amino}-propyl)-4-hydroxy-benzamide) was obtained as a light tan powder (yield: 66%).

### Example 2

### General Procedure for Screening Compounds for Activity

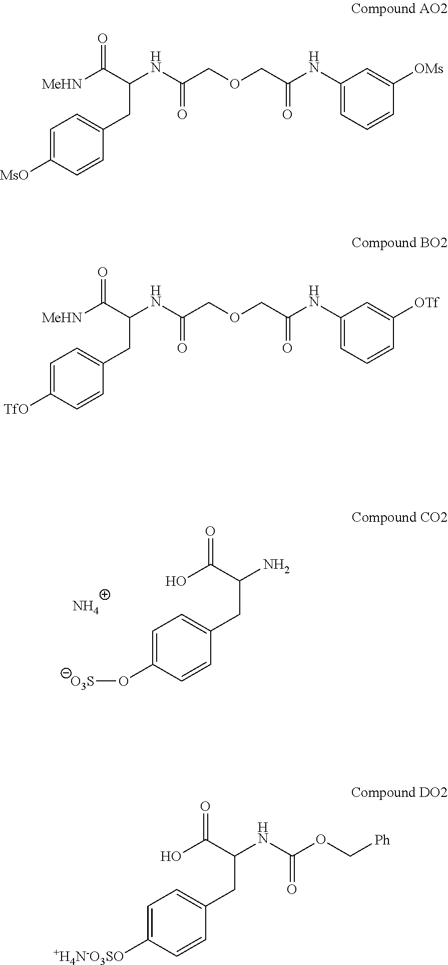

The results of screening the four compounds shown above are representative. Compounds were packaged in 2-5 mg quantities in wells A02-D02 of a 96-deep well polypropylene microplate, tightly sealed with a plastic dimpled lid. The four compounds were handled under slightly different conditions as they fell into two solubility classes. Compounds A02 and B02, insoluble in water, were dissolved in DMSO and tested at a final concentration of 0.5% with 5% DMSO, a concentration of DMSO that has no effect on any of the organisms used in the assays. Compounds C02 and D02, soluble in water, were dissolved in E-pure water, and tested at 0.5% after having been adjusted to the proper buffer condition for the assay. The compounds were tested in three species, Staphylococcus epidermidis, important in human pathology, Colletotrichum acutatum, a plant pathogenic fungus, and Pseudoalteramonas atlantica, a marine biofouling bacterium. The two bacteria were tested using a 96-well multiwell plate protocol, using the fluorescent dye, Syto 13, to quantitate the assay. The fungus was tested in a 35 mm petri dish protocol using spore counts to quantitate the test. Methods for assays testing for activity against Staphylococcus, Pseudoalteramonas, and Colletotrichum species are described in Examples 3, 4, and 5, respectively. Results of the assays are reported in Example 2.

### Example 3

### Inhibition of Adhesion of Cells or Spores to Polystyrene Surfaces

The results tabulated herein were generated using the protocol described in Example 1.

| | Staphylococcus | Pseudoalteramonas | Colletotrichum |
|---|---|---|---|
| Compound | epidermidis | atlantica | acutatum |
| Compound A02 | 22% | ND | −12% |
| Compound B02 | ND | ND | −16% |
| Compound C02 | 6% | ND | −36% |
| Compound D02 | 74% | 32% | −19% |

N.D. Not determined

### Example 4

### 96-Well Plate Assay for Activity Against Staphyloccocus epidermidis

### Reagents Used:

| | | | |
|---|---|---|---|
| 10XPBS | 80 g | NaCl | 1 L |
| | 2 g | KH₂PO₄ | |
| | 11.35 g/21.4 g | Na₂HPO₄/Na₂HPO₄7H₂O | |
| | 2 g | KCl | |
| | pH 7.2 | | |
| SYTO 13 | 5 mM in DMSO | | |

### LB Growth Media

1. S. epidermidis (ATCC #12228) is grown on nutrient agar plates from −70° C. stock, a single colony should be picked and subcultured in LB media for overnight growth at 37° C. shaking 200 rpm.
2. Use 1/100^{th} volume of overnight culture to start new culture, this culture will need to grow for 4-5 hr which will be early log phase of growth.
3. Pour culture into 50 mL conical tubes and centrifuge at 3000 rpm for 10 min at R.T., pour off supernatant and gently re-suspend pellet in 0.5 volume of original culture with 1×PBS buffer.
4. Use this re-suspended culture to determine OD using the Shimadzu spectrophotometer and dilute cells to 1 OD at 600 nm.
5. Experimental controls and samples should be added to wells prior to adding cells, positive control at 2, 1, and 0.1% added to three wells each.
6. Dynex 96 well black solid bottom plates are used for the fluorescence assay. Prep standard curve wells by adding 50 μt of 1×PBS in each well, 15 wells are needed for a 1:1 dilution series starting at 0.5-0.03125 OD of cells in triplicate. Make dilutions and add the Syto 13 probe (1 uM final) fresh just before adding to the wells. The 9 wells below standards need to be saved for background controls, these also will need the probe as well.
7. Working O.D. was 0.0625 OD and Syto 13 probe is 1 μM final, well volume is 100 μt. Once experimental samples have been incubated in the wells for a minimum of 10 min then add cells to each well according to final volume and concentrations.
8. Incubate cells on plate for 25 min then wash on Tecan Plate washer 5 cycles of 300 μL rinse with 1×PBS solution. At last cycle dispense 100 μL of 1×PBS solution and read fluorescence.
9. Excitation is 488 nm and Emission at 509 nm, use the Tecan Microfluor Fluorescein setting.

### Example 5

### 96-Well-Plate Assay for Activity Against Pseudoalteromonas atlantica

### 1. Media and Reagent Preparation:

Marine broth (Defico 2216): Medium prepared according to the manufacture's instructions and autoclaved for 20 min. Let sit for 1-2 days and transfer the clear part to a sterile bottle and store at RT.
80% seawater (Filtered natural seawater (FSW) or artificial seawater (ASW, sigma 5-9883). Autoclave to sterilize and store at RT.
PBS: see Example 3.
1 μM Syto-13: add 1 μl of 5 mM stock (in DMSO) from Molecular Probe (Cat #7575) to every 5 ml PBS.

### 2. Preparation for Cell Material:

Cultured in sterile marine broth in late afternoon (ca. 5-6 pm) one day before assaying. Grown at 26-29° C. in a shaking incubator (200 rpm).

Harvest the cells when the OD₆₀₀ of the culture reaches 0.7-1.0, using centrifugation (3000 rpm, 10 min, RT). Resuspend the cells with 80% seawater to OD between 0.3 to 1.0, then use the cell preparation within an hour.

### 3. Assaying Procedure:

Dynex total-black plate and Nunc total-black plate (with or without coating) work well, but Nunc total-black plate coated is the best.

Make solutions containing ZA double concentrated as intended to use, using 80% seawater.

Add 50 μl of the solutions to each well on the plate. Add 50 μl of 80% seawater to the wells as control.

Then add 50 μl of the cell preparation described above into those wells. Shake the plate briefly to make sure that cells are mixed with ZA solution.

Incubated the plate at RT for 40 min (no shaking is needed).

After incubation, washed the plate with the plate-washer using 80% seawater (program 3, which is a 5 cycle wash), and then add 100 μl of 1 μM Syto-13 in PBS into each well.

Incubate the plate with the dye solution for 15 min in darkness or at least under dim light.

Read the plate with Tecan using the program “Syto-13” (Excitation is 488 nm and Emission at 509 nm). Set the gain at either 100 or optimal.

### Example 6

### Petri Dish Assay for Activity Against Colletotrichum acutatum

### Reagents:

Tap water
50/50 Oatmeal/PDA agar plates
1. C. acutatum was grown on 50/50 oatmeal/PDA agar plates for 6-8 days. Seven days are optimal. Regular inoculations of plates can be made from plate to plate, but new stocks should be brought up from the frozen stock every two months.
   10. Flooded plate with 10-15 ml tap water, pipetting wash gently over the culture to tease the spores loose.
   11. Transferred wash into 15 or 50 ml conical tubes and centrifuge at 3000 rpm for 5 min at R.T., pour off supernatant and vortex pellet in 10 ml of tap water to resuspend pellet.
   12. Counted spores at a 1:100 dilution, using a haemocytometer. Counts generally are 6-8×107 spores/ml. Dilute spores to 2×10⁵ spores/ml; about 35 μl/10 ml tap water. Refrigerate spore suspensions.
   13. For experiment, data points were staggered at 15 min intervals. Set up three Eppendorf tubes. Add 100 μl of test compound made up in e-pure water to the tube. Add 100 μl of spore suspension to the tubes.
   14. Allowed to equilibrate for 15 min. Transfer 100 μl from each tube to a 35 mm Falcon petri dish which has been labeled and marked with a graphic on the bottom to orient the plate. Place the drop in the center of the graphic.
   15. At 55 minutes took 5 images of the spores on the surface of the petri dish.
   16. At 1 hr after the spores were originally put into the Eppendorf tubes, washed spores from petri dish by adding 3 ml of ultra-pure water to each dish and vortex dishes on the microplate shaker at a setting of 900 rpm for 30 seconds. Take an additional 5 images per dish following the same pattern as in step 6. There should be ˜200-500 spores/field.
   17. Counted the spores in the images using the Image Pro software, enter the counts into the Excel template set up for this assay, which also calculates the % inhibition for each experimental point.

### Example 7

### Direct Binding Assay

The virus particles were collected and fixed in 100% (USP) ethanol prior to DART TOF-MS analysis. The closed end of a borosilicate glass capillary tube was immersed in the virion solution and passed through the DART He plasma to obtain mass spectra of virion surface-bound chemistries. Virions subjected to the Direct Binding Assay were also used in focus reduction assays as described above in part b to determine the role of the binding in viral entry activity. The same conditions were used to assess the binding of composition here to bacterial species.

1. Viruses Such as Dengue are Prepared for Infection and Direct Binding Assays as Described Below

a. Viruses and Cells

DENV-1 strain HI-1, DENV-2 strain NG-2, DENV-3 strain H-78, and DENV-4 strain H-42 were obtained from R. Tesh at the World Health Organization Arbovirus Reference Laboratory at the University of Texas at Galveston. Viruses were propagated in the African green monkey kidney epithelial cell line, LLCMK-2, a gift of K. Olsen at Colorado State University. LLCMK-2 cells were grown in DMEM with 10% (v/v) FBS, 2 mM Glutamax, 100 U/ml penicillin G, 100 μg/ml streptomycin and 0.25 μg/ml amphotericin B, at 37° C. with 5% (v/v) CO₂.

b. Focus Forming Unit (FFU) Reduction Assay

LLCMK-2 target cells were seeded at a density of 1×10⁵ cells in each well of a 6-well plate 24 h prior to infection. Approximately 200 FFU of virus were incubated with or without chemistries in serum-free DMEM for 1 h at rt. Virus/chemistry or virus/control mixtures were allowed to infect confluent target cell monolayers for 1 h at 37° C., with rocking every 15 m, after which time the medium was aspirated and overlaid with fresh DMEM/10% (v/v) FBS containing 0.85% (w/v) Sea-Plaque Agarose (Cambrex Bio Science, Rockland, Me.). Cells with agarose overlays were incubated at 4° C. for 20 m to set the agar. Infected cells were then incubated at 37° C. with 5% CO₂ for 3 days (DENV-1, 3 and 4) or 5 days (DENV-2). Infected cultures were fixed with neutral buffered 10% formalin (Formalde-Fresh, Fisher Scientific, Waltham, Mass.) overnight at 4° C. permeabilized with 70% (v/v) ethanol for 20 m at rt, and rinsed with PBS prior to immunostaining Virus foci were detected using supernatant from mouse anti-DENV hybridoma E60 (obtained from M. Diamond at Washington University) followed by horseradish peroxidase-conjugated goat anti-mouse immunoglobulin (Pierce, Rockford, Ill.) and developed using AEC chromogen substrate (Dako, Carpinteria, Calif.). Results were expressed as the average of at least two independent trials with three replicates in each trial.

c. Cytotoxicity Assay

The cytotoxicity of the chemistries was measured by monitoring mitochondrial reductase activity using the TACS™ MTT cell proliferation assay (R&D Systems, Inc., Minneapolis, Minn.) according to the manufacturer's instructions. Dilutions of chemistries in serum-free DMEM were added to confluent monolayers of LLCMK-2 cells in 96-well plates for 1 h at 37° C., similar to the focus forming inhibition assays, and incubated at 37° C. with 5% (v/v) CO₂ for 24 h. Absorbance at 560 nm was measured using a Tecan GeniosPro plate reader (Tecan U S, Durham, N C).

d. Bacterial Preparations

Bacteria or fungi grown as described in Examples 3-6 grown as described below are used to determine the specific interactions of the composition herein with the pathogens.

### Example 8

### Combinatorial Library

A combinatorial library was synthesized containing compounds having structures according to Template 6, shown below.

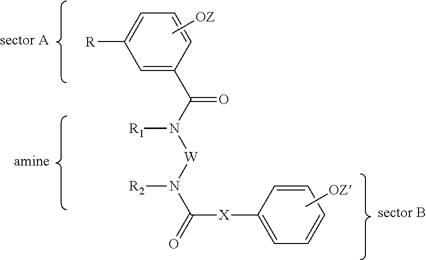

### Template 6

R was either H, Cl, or OMe. Z and Z′ are as defined above. The linker comprised a saturated or unsaturated hydrocarbon tether consisting of between 2 and about 15 carbon atoms, which tether may comprise an aromatic nucleus. The linker can be derived from any diamine. X was a bond or (E)-CH═CH—.

The aromatic nucleus of Sector A was derived from any of the following four methyl benzoates:

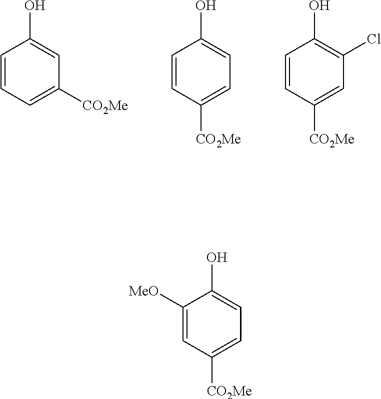

X and the aromatic nucleus of Sector B was derived from any of the following β-phenyl acrylic acids:

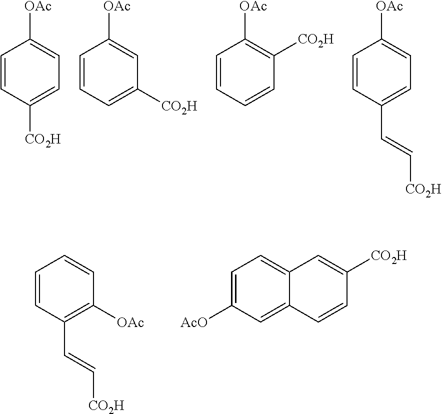

The amine sector was derived from the following diamines: 2,2-dimethyl-1,3-propanediamine, ethylene diamine, cis-1,2-diaminocyclohexane, 1,4-diaminobutane, 1,5-diaminopentane, 2,2′-thiobis(ethylamine), homopiperazine, piperazine, 1,6-hexanediamine, N,N′-dimethylethylenediamine, 1,8-diamino-3,6-dioxaoctane, N,N′-diethyl-2-butene-1,4-diamine, N,N′-diethyl-1,3-propane-diamine, 1,3-diaminopropane, 2,2-dimethyl-1,3-propane diamine, trans-1,4-diaminocyclohexane.

The bisphenol, monosulfate and disulfates were prepared. As shown below, the bisphenol was isolated after deprotection. Alternatively, the bisphenol was subjected to sulfation to produce mono or di-sulfates. Thus, the synthesis provided compounds where Z and Z′ are both H; one of Z and Z′ is H; and one is a sulfate, or both Z and Z′ are sulfate.

Members of this combinatorial library were synthesized as shown below.

### Example 8A

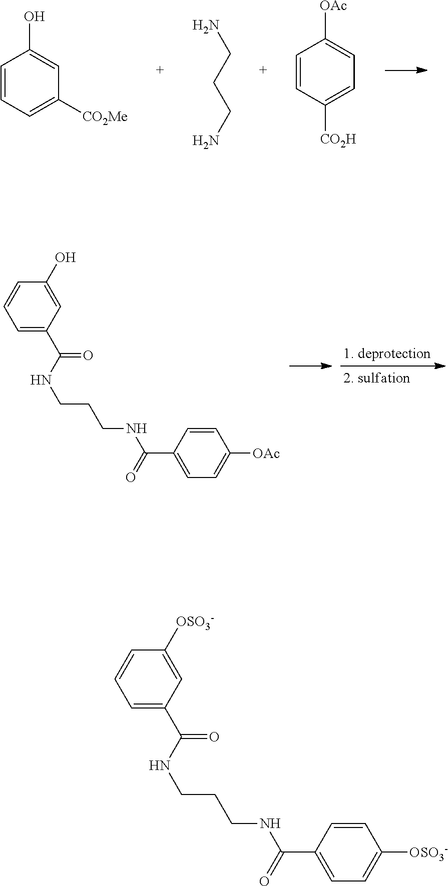

### Example 8B

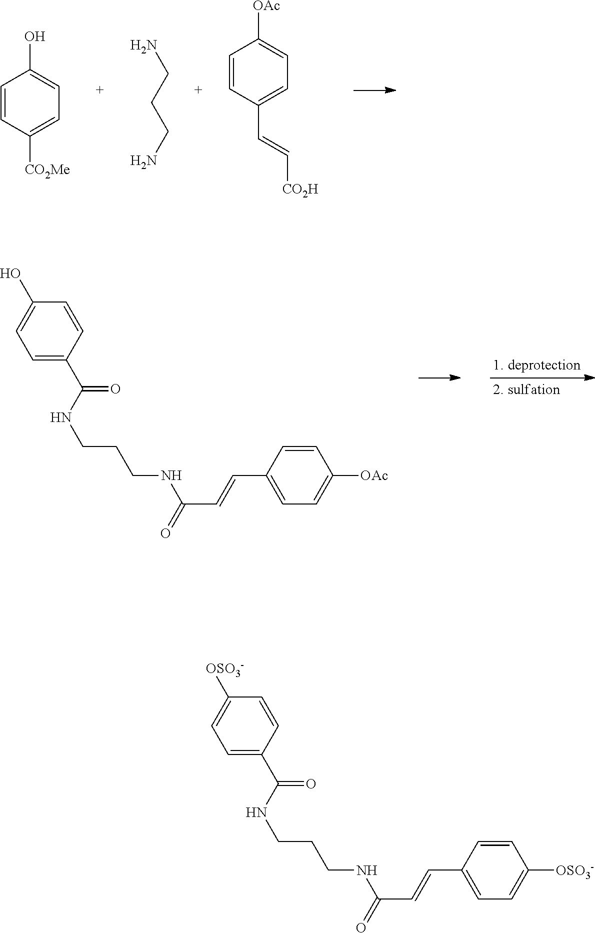

### Example 8C

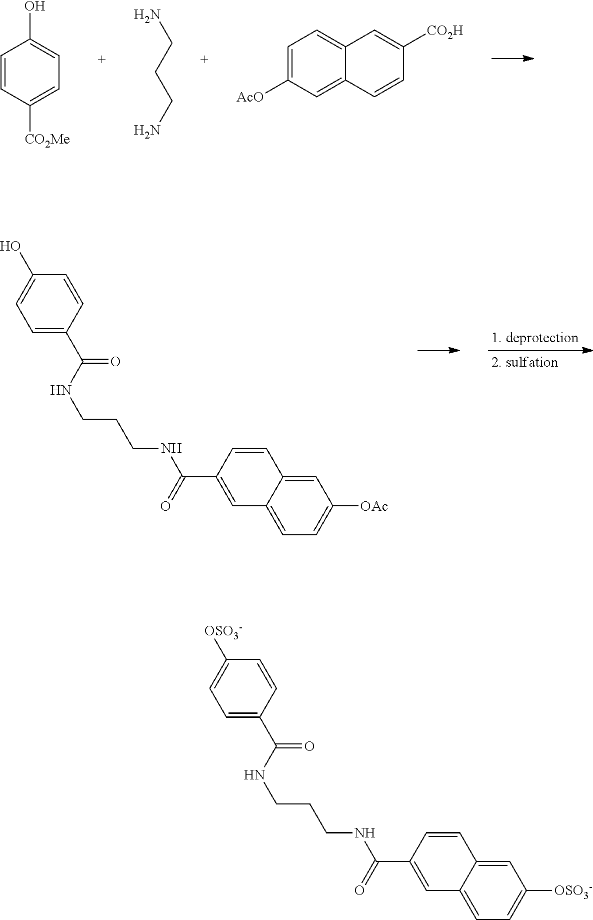

A portion of the general synthetic scheme is depicted below:

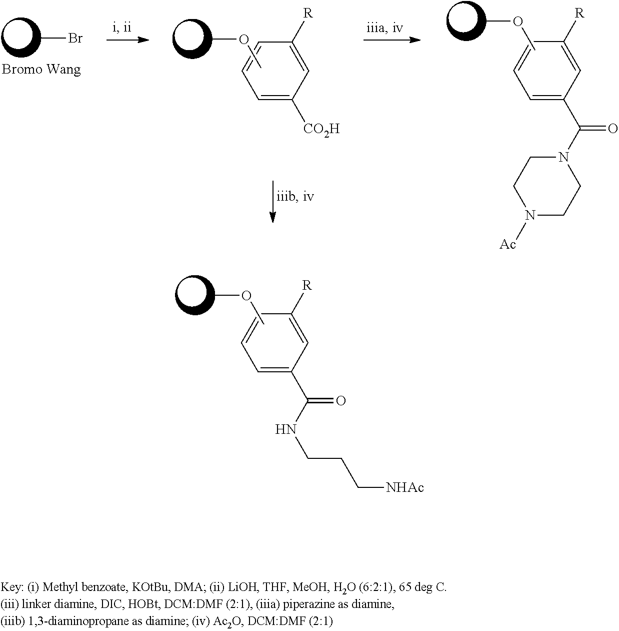

The monosulfates and disulfates prepared are listed below in Table 1, along with biological activity data:

TABLE 1

| | S. aur | P. aer | C. acu | Sector A | Amine | Sector B | Z | Z′ |
|---|---|---|---|---|---|---|---|---|
| 1 | NM | <.015 | NM | ME-P-OH-BN | 2,2-DIMETHYL-1,3-PROPANEDIAMINE | 3-Ac-BA | H | SO3— |
| 2 | NM | <.015 | NM | ME-P-OH-BN | 2,2-DIMETHYL-1,3-PROPANEDIAMINE | 4-Ac-CA | H | SO3— |
| 3 | NM | NM | >.015 | ME-P-OH-BN | 2,2-DIMETHYL-1,3-PROPANEDIAMINE | 2-Ac-CA | SO3— | SO3— |
| 4 | NM | <.015 | NM | ME-P-OH-BN | 2,2-DIMETHYL-1,3-PROPANEDIAMINE | AC SA | H | SO3— |
| 5 | NM | <.015 | NM | ME-P-OH-BN | 2,2-DIMETHYL-1,3-PROPANEDIAMINE | AC SA | SO3— | H |
| 6 | NM | <.015 | NM | ME-P-OH-BN | ETHYLENEDIAMINE | 4-Ac BA | SO3— | H |
| 7 | NM | <.015 | NM | ME-P-OH-BN | CIS-1,2-DIAMINOCYCLOHEXANE | 4-Ac BA | H | SO3— |
| 8 | NM | NM | NM | ME-P-OH-BN | CIS-1,2-DIAMINOCYCLOHEXANE | 3-Ac-BA | SO3— | H |
| 9 | >0.015 | NM | 0.016 | ME-P-OH-BN | CIS-1,2-DIAMINOCYCLOHEXANE | 3-Ac-BA | H | SO3— |
| 10 | NM | <.015 | NM | ME-P-OH-BN | CIS-1,2-DIAMINOCYCLOHEXANE | 2-Ac-CA | H | SO3— |
| 11 | NM | NM | 0.039 | ME-P-OH-BN | CIS-1,2-DIAMINOCYCLOHEXANE | 2-Ac-CA | SO3— | H |
| 12 | NM | <.015 | NM | ME-P-OH-BN | CIS-1,2-DIAMINOCYCLOHEXANE | AC SA | H | SO3— |
| 13 | >0.015 | NM | >.015 | ME-P-OH-BN | CIS-1,2-DIAMINOCYCLOHEXANE | AC SA | SO3— | H |
| 14 | NM | <.015 | NM | ME-P-OH-BN | CIS-1,2-DIAMINOCYCLOHEXANE | 6-Ac-2-NA | H | SO3— |
| 15 | NM | NM | 0.018 | ME-P-OH-BN | CIS-1,2-DIAMINOCYCLOHEXANE | 6-Ac-2-NA | SO3— | H |
| 16 | NM | NM | NM | ME-P-OH-BN | 1,4-DIAMINOBUTANE | 4-Ac BA | SO3— | H |
| 17 | NM | <.015 | NM | ME-P-OH-BN | 1,4-DIAMINOBUTANE | 3-Ac-BA | H | SO3— |
| 18 | >0.015 | NM | NM | ME-P-OH-BN | 1,4-DIAMINOBUTANE | 4-Ac-CA | SO3— | H |
| 19 | NM | <.015 | NM | ME-P-OH-BN | 1,4-DIAMINOBUTANE | 2-Ac-CA | H | SO3— |
| 20 | NM | <.015 | NM | ME-P-OH-BN | 1,4-DIAMINOBUTANE | AC SA | SO3— | SO3— |
| 21 | NM | <.015 | NM | ME-P-OH-BN | 1,4-DIAMINOBUTANE | AC SA | H | SO3— |
| 22 | >0.015 | >.015 | 0.210 | ME-P-OH-BN | 1,4-DIAMINOBUTANE | AC SA | SO3— | H |
| 23 | NM | <.015 | NM | ME-P-OH-BN | TRANS-1,4-DIAMINOCYCLOHEXANE | 3-Ac-BA | H | SO3— |
| 24 | NM | <.015 | NM | ME-P-OH-BN | TRANS-1,4-DIAMINOCYCLOHEXANE | 3-Ac-BA | SO3— | H |
| 25 | NM | <.015 | NM | ME-P-OH-BN | TRANS-1,4-DIAMINOCYCLOHEXANE | AC SA | SO3— | SO3— |
| 26 | NM | <.015 | NM | ME-P-OH-BN | TRANS-1,4-DIAMINOCYCLOHEXANE | AC SA | H | SO3— |
| 27 | NM | NM | 0.013 | ME-P-OH-BN | TRANS-1,4-DIAMINOCYCLOHEXANE | AC SA | SO3— | H |
| 28 | NM | NM | NM | ME-P-OH-BN | 1,5-DIAMINOPENTANE | 4-Ac BA | SO3— | SO3— |
| 29 | NM | <.015 | NM | ME-P-OH-BN | 1,5-DIAMINOPENTANE | 4-Ac BA | H | SO3— |
| 30 | NM | NM | NM | ME-P-OH-BN | 1,5-DIAMINOPENTANE | 4-Ac BA | SO3— | H |
| 31 | NM | NM | NM | ME-P-OH-BN | 1,5-DIAMINOPENTANE | AC SA | SO3— | SO3— |
| 32 | NM | NM | NM | ME-P-OH-BN | 1,5-DIAMINOPENTANE | AC SA | H | SO3— |
| 33 | >0.015 | NM | NM | ME-P-OH-BN | 1,5-DIAMINOPENTANE | AC SA | SO3— | H |
| 34 | >0.015 | NM | NM | ME-P-OH-BN | 2,2′-THIOBIS(ETHYLAMINE) | AC SA | SO3— | H |
| 35 | NM | <.015 | NM | ME-P-OH-BN | 2,2′-THIOBIS(ETHYLAMINE) | AC SA | SO3— | SO3— |
| 36 | NM | <.015 | NM | ME-P-OH-BN | 2,2′-THIOBIS(ETHYLAMINE) | AC SA | H | SO3— |
| 37 | NM | NM | NM | ME-P-OH-BN | HOMOPIPERAZINE | 3-Ac-BA | SO3— | H |
| 38 | NM | >.015 | NM | ME-P-OH-BN | HOMOPIPERAZINE | 4-Ac-CA | SO3— | H |
| 39 | NM | NM | NM | ME-P-OH-BN | HOMOPIPERAZINE | 2-Ac-CA | SO3— | H |
| 40 | NM | >.015 | NM | ME-P-OH-BN | 1,6-HEXANEDIAMINE | 4-Ac BA | SO3— | H |
| 41 | NM | NM | NM | ME-P-OH-BN | 1,6-HEXANEDIAMINE | 3-Ac-BA | SO3— | H |
| 42 | NM | <.015 | NM | ME-P-OH-BN | 1,6-HEXANEDIAMINE | 4-Ac-CA | H | SO3— |
| 43 | NM | <.015 | NM | ME-P-OH-BN | 1,6-HEXANEDIAMINE | 2-Ac-CA | H | SO3— |
| 44 | NM | NM | >.015 | ME-P-OH-BN | 1,6-HEXANEDIAMINE | AC SA | SO3— | H |
| 45 | NM | <.015 | NM | ME-P-OH-BN | 1,6-HEXANEDIAMINE | AC SA | H | SO3— |
| 46 | NM | NM | NM | ME-P-OH-BN | N,N′-DIMETHYLETHYLENEDIAMINE | 4-Ac BA | SO3— | H |
| 47 | NM | NM | NM | ME-P-OH-BN | N,N′-DIMETHYLETHYLENEDIAMINE | 3-Ac-BA | SO3— | H |
| 48 | NM | NM | NM | ME-P-OH-BN | N,N′-DIMETHYLETHYLENEDIAMINE | 4-Ac-CA | SO3— | H |
| 49 | NM | NM | NM | ME-P-OH-BN | N,N′-DIETHYL-1,3-PROPANEDIAMINE | 4-Ac BA | SO3— | H |
| 50 | NM | NM | NM | ME-P-OH-BN | N,N′-DIETHYL-1,3-PROPANEDIAMINE | 3-Ac-BA | SO3— | H |
| 51 | NM | NM | NM | ME-P-OH-BN | N,N′-DIETHYL-1,3-PROPANEDIAMINE | 4-Ac-CA | SO3— | H |
| 52 | NM | NM | NM | ME-P-OH-BN | N,N′-DIETHYL-1,3-PROPANEDIAMINE | 2-Ac-CA | SO3— | H |
| 53 | NM | NM | NM | ME-P-OH-BN | N,N′-DIETHYL-1,3-PROPANEDIAMINE | AC SA | SO3— | H |
| 54 | NM | NM | NM | ME-P-OH-BN | 1,8-DIAMINO-3,6-DIOXAOCTANE | 3-Ac-BA | SO3— | H |
| 55 | NM | NM | NM | ME-P-OH-BN | 1,8-DIAMINO-3,6-DIOXAOCTANE | 4-Ac-CA | SO3— | H |
| 56 | NM | NM | NM | ME-P-OH-BN | N,N′-DIETHYL-2-BUTENE-1,4-DIAMINE | 4-Ac BA | H | SO3— |
| 57 | >0.015 | NM | NM | ME-P-OH-BN | N,N′-DIETHYL-2-BUTENE-1,4-DIAMINE | 4-Ac BA | SO3— | H |
| 58 | NM | <.015 | NM | ME-P-OH-BN | N,N′-DIETHYL-2-BUTENE-1,4-DIAMINE | 3-Ac-BA | SO3— | SO3— |
| 59 | >0.015 | NM | NM | ME-P-OH-BN | N,N′-DIETHYL-2-BUTENE-1,4-DIAMINE | 3-Ac-BA | SO3— | H |
| 60 | NM | NM | NM | ME-P-OH-BN | N,N′-DIETHYL-2-BUTENE-1,4-DIAMINE | 4-Ac-CA | H | SO3— |
| 61 | NM | NM | NM | ME-P-OH-BN | N,N′-DIETHYL-2-BUTENE-1,4-DIAMINE | 4-Ac-CA | SO3— | H |
| 62 | NM | NM | NM | ME-P-OH-BN | N,N′-DIETHYL-2-BUTENE-1,4-DIAMINE | 2-Ac-CA | H | SO3— |
| 63 | NM | NM | NM | ME-P-OH-BN | N,N′-DIETHYL-2-BUTENE-1,4-DIAMINE | 2-Ac-CA | SO3— | H |
| 64 | NM | NM | NM | ME-P-OH-BN | N,N′-DIETHYL-2-BUTENE-1,4-DIAMINE | AC SA | H | SO3— |
| 65 | >0.015 | NM | NM | ME-P-OH-BN | N,N′-DIETHYL-2-BUTENE-1,4-DIAMINE | AC SA | SO3— | H |
| 66 | NM | NM | NM | ME-3-CL-4-OH-BN | 1,3-DIAMINOPROPANE | 4-Ac BA | SO3— | H |
| 67 | NM | <.015 | NM | ME-3-CL-4-OH-BN | 1,3-DIAMINOPROPANE | 4-Ac BA | H | SO3— |
| 68 | >0.015 | NM | NM | ME-3-CL-4-OH-BN | 1,3-DIAMINOPROPANE | 3-Ac-BA | SO3— | H |
| 69 | NM | <.015 | NM | ME-3-CL-4-OH-BN | 1,3-DIAMINOPROPANE | 3-Ac-BA | H | SO3— |
| 70 | NM | <.015 | NM | ME-3-CL-4-OH-BN | 1,3-DIAMINOPROPANE | 4-Ac-CA | H | SO3— |
| 71 | NM | <.015 | NM | ME-3-CL-4-OH-BN | 1,3-DIAMINOPROPANE | AC SA | SO3— | SO3— |
| 72 | NM | <.015 | NM | ME-3-CL-4-OH-BN | 1,3-DIAMINOPROPANE | AC SA | H | SO3— |
| 73 | NM | NM | NM | ME-3-CL-4-OH-BN | 1,3-DIAMINOPROPANE | AC SA | SO3— | H |
| 74 | NM | <.015 | 0.354 | ME-3-CL-4-OH-BN | 1,3-DIAMINOPROPANE | AC SA | H | SO3— |
| 75 | NM | <.015 | 0.024 | ME-3-CL-4-OH-BN | 1,3-DIAMINOPROPANE | AC SA | SO3— | H |
| 76 | NM | NM | >.015 | ME-3-CL-4-OH-BN | 2,2-DIMETHYL-1,3-PROPANEDIAMINE | 4-Ac BA | SO3— | H |
| 77 | NM | NM | NM | ME-3-CL-4-OH-BN | 2,2-DIMETHYL-1,3-PROPANEDIAMINE | 3-Ac-BA | SO3— | H |
| 78 | NM | NM | NM | ME-3-CL-4-OH-BN | 2,2-DIMETHYL-1,3-PROPANEDIAMINE | 4-Ac-CA | SO3— | H |
| 79 | NM | NM | NM | ME-3-CL-4-OH-BN | 1,4-DIAMINOBUTANE | 3-Ac-BA | SO3— | H |
| 80 | NM | NM | NM | ME-3-CL-4-OH-BN | 1,4-DIAMINOBUTANE | 4-Ac-CA | SO3— | H |
| 81 | >0.015 | NM | NM | ME-3-CL-4-OH-BN | TRANS-1,4-DIAMINOCYCLOHEXANE | 3-Ac-BA | SO3— | H |
| 82 | NM | NM | NM | ME-3-CL-4-OH-BN | TRANS-1,4-DIAMINOCYCLOHEXANE | 4-Ac-CA | SO3— | H |
| 83 | NM | NM | NM | ME-3-CL-4-OH-BN | TRANS-1,4-DIAMINOCYCLOHEXANE | 2-Ac-CA | SO3— | H |
| 84 | NM | NM | NM | ME-3-CL-4-OH-BN | 1,5-DIAMINOPENTANE | 4-Ac BA | SO3— | H |
| 85 | NM | NM | NM | ME-3-CL-4-OH-BN | 1,5-DIAMINOPENTANE | 3-Ac-BA | SO3— | H |
| 86 | >0.015 | NM | 0.019 | ME-3-CL-4-OH-BN | 1,5-DIAMINOPENTANE | 4-Ac-CA | SO3— | H |
| 87 | NM | >.015 | 0.029 | ME-3-CL-4-OH-BN | 1,5-DIAMINOPENTANE | 4-Ac-CA | H | SO3— |
| 88 | NM | NM | 0.009 | ME-3-CL-4-OH-BN | 1,5-DIAMINOPENTANE | 2-Ac-CA | SO3— | H |
| 89 | NM | NM | NM | ME-3-CL-4-OH-BN | 2,2′-THIOBIS(ETHYLAMINE) | 4-Ac BA | SO3— | H |
| 90 | >0.015 | NM | NM | ME-3-CL-4-OH-BN | 2,2′-THIOBIS(ETHYLAMINE) | 3-Ac-BA | SO3— | H |
| 91 | NM | NM | NM | ME-3-CL-4-OH-BN | 2,2′-THIOBIS(ETHYLAMINE) | 4-Ac-CA | SO3— | H |
| 92 | NM | NM | NM | ME-3-CL-4-OH-BN | HOMOPIPERAZINE | 4-Ac-CA | SO3— | H |
| 93 | NM | NM | NM | ME-3-CL-4-OH-BN | HOMOPIPERAZINE | 2-Ac-CA | H | SO3— |
| 94 | NM | NM | NM | ME-3-CL-4-OH-BN | HOMOPIPERAZINE | 2-Ac-CA | SO3— | H |
| 95 | NM | NM | NM | ME-3-CL-4-OH-BN | 1,6-HEXANEDIAMINE | 4-Ac BA | SO3— | H |
| 96 | >0.015 | NM | NM | ME-3-CL-4-OH-BN | 1,6-HEXANEDIAMINE | 3-Ac-BA | SO3— | H |
| 97 | NM | NM | 0.016 | ME-3-CL-4-OH-BN | 1,6-HEXANEDIAMINE | 4-Ac-CA | SO3— | H |
| 98 | NM | NM | 0.065 | ME-3-CL-4-OH-BN | N,N′-DIMETHYLETHYLENEDIAMINE | 4-Ac-CA | SO3— | H |
| 99 | NM | NM | >.015 | ME-3-CL-4-OH-BN | N,N′-DIETHYL-1,3-PROPANEDIAMINE | 4-Ac BA | H | SO3— |
| 100 | NM | NM | NM | ME-3-CL-4-OH-BN | N,N′-DIETHYL-1,3-PROPANEDIAMINE | 4-Ac BA | SO3— | H |
| 101 | NM | NM | NM | ME-3-CL-4-OH-BN | N,N′-DIETHYL-1,3-PROPANEDIAMINE | 3-Ac-BA | SO3— | H |
| 102 | NM | NM | NM | ME-3-CL-4-OH-BN | N,N′-DIETHYL-1,3-PROPANEDIAMINE | 3-Ac-BA | H | SO3— |
| 103 | >0.015 | NM | NM | ME-3-CL-4-OH-BN | N,N′-DIETHYL-1,3-PROPANEDIAMINE | 4-Ac-CA | SO3— | H |
| 104 | NM | NM | NM | ME-3-CL-4-OH-BN | N,N′-DIETHYL-1,3-PROPANEDIAMINE | 4-Ac-CA | H | SO3— |
| 105 | NM | NM | NM | ME-3-CL-4-OH-BN | N,N′-DIETHYL-1,3-PROPANEDIAMINE | 2-Ac-CA | SO3— | H |
| 106 | NM | NM | NM | ME-3-CL-4-OH-BN | N,N′-DIETHYL-1,3-PROPANEDIAMINE | 2-Ac-CA | H | SO3— |
| 107 | >0.015 | NM | NM | ME-3-CL-4-OH-BN | N,N′-DIETHYL-1,3-PROPANEDIAMINE | AC SA | SO3— | H |
| 108 | NM | NM | NM | ME-3-CL-4-OH-BN | N,N′-DIETHYL-1,3-PROPANEDIAMINE | AC SA | H | SO3— |
| 109 | NM | NM | 0.003 | ME-3-CL-4-OH-BN | N,N′-DIETHYL-1,3-PROPANEDIAMINE | 6-Ac-2-NA | SO3— | H |
| 110 | NM | NM | NM | ME-3-CL-4-OH-BN | 1,8-DIAMINO-3,6-DIOXAOCTANE | 4-Ac BA | SO3— | H |
| 111 | >0.015 | NM | NM | ME-3-CL-4-OH-BN | 1,8-DIAMINO-3,6-DIOXAOCTANE | 3-Ac-BA | SO3— | H |
| 112 | NM | NM | NM | ME-3-CL-4-OH-BN | 1,8-DIAMINO-3,6-DIOXAOCTANE | 2-Ac-CA | SO3— | SO3— |
| 113 | NM | NM | NM | ME-3-CL-4-OH-BN | 1,8-DIAMINO-3,6-DIOXAOCTANE | 2-Ac-CA | SO3— | H |
| 114 | NM | <.015 | NM | ME-3-CL-4-OH-BN | 1,8-DIAMINO-3,6-DIOXAOCTANE | 2-Ac-CA | H | SO3— |
| 115 | NM | NM | >.015 | ME-3-CL-4-OH-BN | N,N′-DIETHYL-2-BUTENE-1,4-DIAMINE | 4-Ac-CA | SO3— | H |
| 116 | NM | NM | 0.032 | ME-3-CL-4-OH-BN | N,N′-DIETHYL-2-BUTENE-1,4-DIAMINE | 2-Ac-CA | SO3— | H |
| 117 | >0.015 | NM | NM | ME-3-OH-BN | 2,2-DIMETHYL-1,3-PROPANEDIAMINE | 4-Ac BA | SO3— | H |
| 118 | NM | NM | NM | ME-3-OH-BN | 2,2-DIMETHYL-1,3-PROPANEDIAMINE | 3-Ac-BA | SO3— | H |
| 119 | NM | <.015 | NM | ME-3-OH-BN | 2,2-DIMETHYL-1,3-PROPANEDIAMINE | 4-Ac-CA | H | SO3— |
| 120 | NM | 0.015 | NM | ME-3-OH-BN | 2,2-DIMETHYL-1,3-PROPANEDIAMINE | AC SA | SO3— | SO3— |
| 121 | >0.015 | NM | NM | ME-3-OH-BN | 2,2-DIMETHYL-1,3-PROPANEDIAMINE | AC SA | H | SO3— |
| 122 | >0.015 | NM | 0.056 | ME-3-OH-BN | 2,2-DIMETHYL-1,3-PROPANEDIAMINE | AC SA | SO3— | H |
| 123 | >0.015 | NM | NM | ME-3-OH-BN | ETHYLENEDIAMINE | 3-Ac-BA | SO3— | H |
| 124 | NM | NM | NM | ME-3-OH-BN | ETHYLENEDIAMINE | 3-Ac-BA | H | SO3— |
| 125 | NM | <.015 | NM | ME-3-OH-BN | ETHYLENEDIAMINE | 3-Ac-BA | SO3— | SO3— |
| 126 | NM | <.015 | NM | ME-3-OH-BN | ETHYLENEDIAMINE | AC SA | H | SO3— |
| 127 | NM | <.015 | NM | ME-3-OH-BN | ETHYLENEDIAMINE | AC SA | SO3— | H |
| 128 | NM | <.015 | NM | ME-3-OH-BN | CIS-1,2-DIAMINOCYCLOHEXANE | 3-Ac-BA | SO3— | SO3— |
| 129 | NM | NM | NM | ME-3-OH-BN | CIS-1,2-DIAMINOCYCLOHEXANE | 3-Ac-BA | SO3— | H |
| 130 | NM | >.015 | NM | ME-3-OH-BN | CIS-1,2-DIAMINOCYCLOHEXANE | AC SA | SO3— | SO3— |
| 131 | NM | >.015 | NM | ME-3-OH-BN | CIS-1,2-DIAMINOCYCLOHEXANE | AC SA | SO3— | SO3— |
| 132 | NM | NM | >.015 | ME-3-OH-BN | CIS-1,2-DIAMINOCYCLOHEXANE | AC SA | SO3— | H |
| 133 | NM | NM | NM | ME-3-OH-BN | 1,4-DIAMINOBUTANE | 3-Ac-BA | SO3— | H |
| 134 | NM | NM | NM | ME-3-OH-BN | 1,4-DIAMINOBUTANE | AC SA | SO3— | H |
| 135 | NM | NM | NM | ME-3-OH-BN | 1,4-DIAMINOBUTANE | AC SA | H | SO3— |
| 136 | NM | 0.015 | NM | ME-3-OH-BN | TRANS-1,4-DIAMINOCYCLOHEXANE | AC SA | H | SO3— |
| 137 | NM | NM | NM | ME-3-OH-BN | TRANS-1,4-DIAMINOCYCLOHEXANE | AC SA | SO3— | H |
| 138 | >0.015 | NM | NM | ME-3-OH-BN | 1,5-DIAMINOPENTANE | 4-Ac BA | SO3— | H |
| 139 | NM | NM | NM | ME-3-OH-BN | 1,5-DIAMINOPENTANE | 3-Ac-BA | SO3— | H |
| 140 | NM | NM | NM | ME-3-OH-BN | 2,2′-THIOBIS(ETHYLAMINE) | 3-Ac-BA | H | SO3— |
| 141 | NM | NM | NM | ME-3-OH-BN | 2,2′-THIOBIS(ETHYLAMINE) | AC SA | SO3— | SO3— |
| 142 | NM | NM | NM | ME-3-OH-BN | 2,2′-THIOBIS(ETHYLAMINE) | AC SA | SO3— | H |
| 143 | NM | <.015 | NM | ME-3-OH-BN | 2,2′-THIOBIS(ETHYLAMINE) | AC SA | H | SO3— |
| 144 | NM | <.015 | NM | ME-3-OH-BN | 1,6-HEXANEDIAMINE | 3-Ac-BA | SO3— | H |
| 145 | NM | >.015 | NM | ME-3-OH-BN | 1,6-HEXANEDIAMINE | 3-Ac-BA | H | SO3— |
| 146 | NM | NM | NM | ME-3-OH-BN | 1,6-HEXANEDIAMINE | 4-Ac-CA | H | SO3— |
| 147 | NM | NM | NM | ME-3-OH-BN | 1,6-HEXANEDIAMINE | 4-Ac-CA | SO3— | H |
| 148 | NM | NM | NM | ME-3-OH-BN | 1,6-HEXANEDIAMINE | AC SA | SO3— | SO3— |
| 149 | NM | NM | 0.015 | ME-3-OH-BN | 1,6-HEXANEDIAMINE | AC SA | H | SO3— |
| 150 | NM | NM | NM | ME-3-OH-BN | 1,6-HEXANEDIAMINE | AC SA | SO3— | H |
| 151 | NM | NM | NM | ME-3-OH-BN | N,N′-DIMETHYLETHYLENEDIAMINE | 2-Ac-CA | H | SO3— |
| 152 | NM | NM | NM | ME-3-OH-BN | N,N′-DIMETHYLETHYLENEDIAMINE | 2-Ac-CA | SO3— | H |
| 153 | NM | >.015 | NM | ME-3-OH-BN | N,N′-DIETHYL-1,3-PROPANEDIAMINE | 4-Ac BA | SO3— | SO3— |
| 154 | NM | NM | 0.199 | ME-3-OH-BN | N,N′-DIETHYL-1,3-PROPANEDIAMINE | 4-Ac BA | SO3— | H |
| 155 | >0.015 | NM | NM | ME-3-OH-BN | N,N′-DIETHYL-1,3-PROPANEDIAMINE | 4-Ac-CA | SO3— | H |
| 156 | NM | NM | NM | ME-3-OH-BN | N,N′-DIETHYL-1,3-PROPANEDIAMINE | 2-Ac-CA | SO3— | H |
| 157 | NM | NM | NM | ME-3-OH-BN | N,N′-DIETHYL-1,3-PROPANEDIAMINE | AC SA | SO3— | H |
| 158 | >0.015 | NM | NM | ME-3-OH-BN | 1,8-DIAMINO-3,6-DIOXAOCTANE | 4-Ac BA | SO3— | H |
| 159 | NM | NM | NM | ME-3-OH-BN | 1,8-DIAMINO-3,6-DIOXAOCTANE | 3-Ac-BA | H | SO3— |
| 160 | NM | NM | NM | ME-3-OH-BN | N,N′-DIETHYL-2-BUTENE-1,4-DIAMINE | 4-Ac BA | H | SO3— |
| 161 | NM | NM | NM | ME-3-OH-BN | N,N′-DIETHYL-2-BUTENE-1,4-DIAMINE | 4-Ac BA | SO3— | H |
| 162 | NM | NM | NM | ME-3-OH-BN | N,N′-DIETHYL-2-BUTENE-1,4-DIAMINE | 3-Ac-BA | SO3— | SO3— |
| 163 | NM | NM | NM | ME-3-OH-BN | N,N′-DIETHYL-2-BUTENE-1,4-DIAMINE | 3-Ac-BA | SO3— | H |
| 164 | NM | <.015 | NM | ME-3-OH-BN | N,N′-DIETHYL-2-BUTENE-1,4-DIAMINE | 4-Ac-CA | SO3— | SO3— |
| 165 | NM | NM | NM | ME-3-OH-BN | N,N′-DIETHYL-2-BUTENE-1,4-DIAMINE | 4-Ac-CA | SO3— | H |
| 166 | NM | NM | NM | ME-3-OH-BN | N,N′-DIETHYL-2-BUTENE-1,4-DIAMINE | 2-Ac-CA | SO3— | H |
| 167 | NM | NM | NM | ME-3-OH-BN | N,N′-DIETHYL-2-BUTENE-1,4-DIAMINE | 2-Ac-CA | H | SO3— |
| 168 | NM | NM | NM | ME-3-OH-BN | N,N′-DIETHYL-2-BUTENE-1,4-DIAMINE | AC SA | H | SO3— |
| 169 | >0.015 | NM | NM | ME-3-OH-BN | N,N′-DIETHYL-2-BUTENE-1,4-DIAMINE | AC SA | SO3— | H |
| 170 | NM | NM | NM | ME VAN | 1,3-DIAMINOPROPANE | AC SA | SO3— | H |
| 171 | NM | NM | NM | ME VAN | 2,2-DIMETHYL-1,3-PROPANEDIAMINE | 4-Ac BA | SO3— | H |
| 172 | NM | NM | NM | ME VAN | 2,2-DIMETHYL-1,3-PROPANEDIAMINE | 3-Ac-BA | SO3— | H |
| 173 | NM | NM | 0.015 | ME VAN | 2,2-DIMETHYL-1,3-PROPANEDIAMINE | 4-Ac-CA | SO3— | H |
| 174 | NM | NM | NM | ME VAN | 2,2-DIMETHYL-1,3-PROPANEDIAMINE | 2-Ac-CA | H | SO3— |
| 175 | NM | NM | 0.097 | ME VAN | 2,2-DIMETHYL-1,3-PROPANEDIAMINE | 2-Ac-CA | SO3— | H |
| 176 | NM | NM | 0.046 | ME VAN | 2,2-DIMETHYL-1,3-PROPANEDIAMINE | AC SA | SO3— | H |
| 177 | NM | NM | NM | ME VAN | ETHYLENEDIAMINE | 3-Ac-BA | H | SO3— |
| 178 | >0.015 | NM | >.015 | ME VAN | ETHYLENEDIAMINE | 3-Ac-BA | SO3— | H |
| 179 | NM | NM | 0.065 | ME VAN | ETHYLENEDIAMINE | AC SA | SO3— | H |
| 180 | NM | NM | NM | ME VAN | CIS-1,2-DIAMINOCYCLOHEXANE | 4-Ac BA | H | SO3— |
| 181 | NM | NM | 0.045 | ME VAN | CIS-1,2-DIAMINOCYCLOHEXANE | 4-Ac BA | SO3— | H |
| 182 | NM | NM | NM | ME VAN | CIS-1,2-DIAMINOCYCLOHEXANE | 4-Ac-CA | SO3— | H |
| 183 | NM | NM | NM | ME VAN | CIS-1,2-DIAMINOCYCLOHEXANE | 2-Ac-CA | SO3— | H |
| 184 | NM | NM | NM | ME VAN | TRANS-1,4-DIAMINOCYCLOHEXANE | 4-Ac BA | SO3— | H |
| 185 | >0.015 | NM | NM | ME VAN | TRANS-1,4-DIAMINOCYCLOHEXANE | 3-Ac-BA | SO3— | SO3— |
| 186 | NM | NM | NM | ME VAN | TRANS-1,4-DIAMINOCYCLOHEXANE | 3-Ac-BA | SO3— | H |
| 187 | >0.015 | NM | 0.012 | ME VAN | TRANS-1,4-DIAMINOCYCLOHEXANE | 4-Ac-CA | SO3— | H |
| 188 | >0.015 | NM | NM | ME VAN | TRANS-1,4-DIAMINOCYCLOHEXANE | AC SA | SO3— | H |
| 189 | NM | NM | NM | ME VAN | 1,5-DIAMINOPENTANE | 3-Ac-BA | H | SO3— |
| 190 | NM | NM | NM | ME VAN | 1,5-DIAMINOPENTANE | 3-Ac-BA | SO3— | H |
| 191 | NM | >.015 | NM | ME VAN | 1,5-DIAMINOPENTANE | 4-Ac-CA | H | SO3— |
| 192 | NM | NM | NM | ME VAN | 1,5-DIAMINOPENTANE | 2-Ac-CA | SO3— | H |
| 193 | >0.015 | NM | NM | ME VAN | 1,5-DIAMINOPENTANE | AC SA | SO3— | H |
| 194 | NM | NM | 0.013 | ME VAN | 1,5-DIAMINOPENTANE | AC SA | SO3— | H |
| 195 | NM | >.015 | 0.011 | ME VAN | 1,5-DIAMINOPENTANE | AC SA | H | SO3— |
| 196 | NM | >.015 | NM | ME VAN | 2,2′-THIOBIS(ETHYLAMINE) | AC SA | H | SO3— |
| 197 | NM | <.015 | NM | ME VAN | 2,2′-THIOBIS(ETHYLAMINE) | AC SA | SO3— | H |
| 198 | NM | NM | 0.186 | ME VAN | 2,2′-THIOBIS(ETHYLAMINE) | AC SA | SO3— | H |
| 199 | >.015 | NM | NM | ME VAN | 1,6-HEXANEDIAMINE | 4-Ac BA | SO3— | SO3— |
| 200 | NM | NM | NM | ME VAN | 1,6-HEXANEDIAMINE | 4-Ac BA | SO3— | H |
| 201 | NM | NM | NM | ME VAN | 1,6-HEXANEDIAMINE | 3-Ac-BA | SO3— | H |
| 202 | NM | NM | 0.01 | ME VAN | 1,6-HEXANEDIAMINE | 2-Ac-CA | SO3— | H |
| 203 | >.015 | NM | NM | ME VAN | N,N′-DIMETHYLETHYLENEDIAMINE | 3-Ac-BA | SO3— | H |
| 204 | NM | NM | 0.064 | ME VAN | N,N′-DIMETHYLETHYLENEDIAMINE | 4-Ac-CA | SO3— | H |
| 205 | NM | NM | >.015 | ME VAN | N,N′-DIETHYL-1,3-PROPANEDIAMINE | 4-Ac BA | SO3— | H |
| 206 | >.015 | NM | NM | ME VAN | N,N′-DIETHYL-1,3-PROPANEDIAMINE | 4-Ac-CA | SO3— | H |
| 207 | NM | NM | NM | ME VAN | N,N′-DIETHYL-1,3-PROPANEDIAMINE | 2-Ac-CA | SO3— | H |
| 208 | >.015 | NM | NM | ME VAN | N,N′-DIETHYL-2-BUTENE-1,4-DIAMINE | 4-Ac BA | SO3— | H |
| 209 | NM | NM | NM | ME VAN | N,N′-DIETHYL-2-BUTENE-1,4-DIAMINE | 3-Ac-BA | SO3— | H |
| 210 | >.015 | <0.015 | >.015 | ME VAN | N,N′-DIETHYL-2-BUTENE-1,4-DIAMINE | 4-Ac-CA | SO3— | SO3— |
| 211 | >.015 | NM | NM | ME VAN | N,N′-DIETHYL-2-BUTENE-1,4-DIAMINE | 4-Ac-CA | SO3— | H |
| 212 | >.015 | NM | NM | ME VAN | N,N′-DIETHYL-2-BUTENE-1,4-DIAMINE | 2-Ac-CA | H | SO3— |
| 213 | NM | NM | NM | ME VAN | N,N′-DIETHYL-2-BUTENE-1,4-DIAMINE | 2-Ac-CA | SO3— | H |
| 214 | NM | <.015 | NM | ME VAN | N,N′-DIETHYL-2-BUTENE-1,4-DIAMINE | AC SA | H | SO3— |
| 215 | >.015 | NM | NM | ME VAN | N,N′-DIETHYL-2-BUTENE-1,4-DIAMINE | AC SA | SO3— | H |
| 216 | NM | NM | NM | ME VAN | N,N′-DIETHYL-2-BUTENE-1,4-DIAMINE | 6-Ac-2-NA | SO3— | H |
| 217 | NM | NM | NM | ME-P-OH-BN | PIPERAZINE | 4-Ac BA | SO3— | H |
| 218 | NM | <.015 | NM | ME-P-OH-BN | PIPERAZINE | 4-Ac-CA | H | SO3— |
| 219 | NM | NM | NM | ME-P-OH-BN | PIPERAZINE | 4-Ac-CA | SO3— | H |
| 220 | NM | NM | NM | ME-3-CL-4-OH-BN | PIPERAZINE | 3-Ac-BA | SO3— | H |
| 221 | NM | NM | NM | ME-3-CL-4-OH-BN | PIPERAZINE | 4-Ac-CA | SO3— | H |
| 222 | NM | >.015 | NM | ME-3-CL-4-OH-BN | PIPERAZINE | 4-Ac-CA | H | SO3— |
| 223 | NM | NM | NM | ME-3-CL-4-OH-BN | PIPERAZINE | 2-Ac-CA | SO3— | H |
| 224 | NM | <.015 | NM | ME-3-CL-4-OH-BN | PIPERAZINE | 2-Ac-CA | H | SO3— |
| 225 | NM | <.015 | NM | ME-3-CL-4-OH-BN | PIPERAZINE | 2-Ac-CA | SO3— | SO3— |
| 226 | NM | NM | NM | ME-3-CL-4-OH-BN | PIPERAZINE | 4-Ac-3- | SO3— | H |
| | | | | | | OMe CA | | |
| 227 | NM | <.015 | NM | ME-3-OH-BN | PIPERAZINE | 4-Ac-CA | H | SO3— |
| 228 | NM | NM | NM | ME-3-OH-BN | PIPERAZINE | 4-Ac-CA | SO3— | H |
| 229 | NM | <.015 | NM | ME-3-OH-BN | PIPERAZINE | 2-Ac-CA | SO3— | H |
| 230 | NM | <.015 | NM | ME-3-OH-BN | PIPERAZINE | 2-Ac-CA | H | SO3— |
| 231 | NM | <.015 | NM | ME-3-OH-BN | PIPERAZINE | 2-Ac-CA | H | SO3— |
| 232 | NM | >.015 | NM | ME-3-OH-BN | PIPERAZINE | 4-Ac-3- | H | SO3— |
| | | | | | | OMe CA | | |
| 233 | NM | NM | NM | ME-3-OH-BN | PIPERAZINE | 4-Ac-3- | SO3— | H |
| | | | | | | OMe CA | | |
| 234 | NM | <.015 | NM | ME-3-OH-BN | PIPERAZINE | N,O- | SO3— | SO3— |
| | | | | | | DIACTYR | | |
| 235 | NM | <.015 | NM | ME VAN | PIPERAZINE | 4-Ac-CA | SO3— | SO3— |
| 236 | NM | <.015 | NM | ME VAN | PIPERAZINE | 4-Ac-CA | H | SO3— |
| 237 | NM | NM | NM | ME VAN | PIPERAZINE | 4-Ac-CA | SO3— | H |
| 238 | NM | >.015 | NM | ME VAN | PIPERAZINE | 4-Ac-3- | SO3— | H |
| | | | | | | OMe CA | | |

Abbreviations
Me-p-OH-Bn = METHYL P-HYDROXYBENZOATE
Me-3-Cl-4-OH-Bn = METHYL 3-CHLORO-4-HYDROXYBENZOATE
Me-3-OH-Bn = METHYL 3-HYDROXYBENZOATE
Me van = METHYL VANILLATE
3-Ac BA = 3-ACETOXYBENZOIC ACID
4-Ac CA = 4-ACETOXYCINNAMIC ACID
2-Ac CA = 2-ACETOXYCINNAMIC ACID
Ac SA = ACETYLSALICYLIC ACID
4-Ac BA = 4-ACETOXYBENZOIC ACID
6-Ac-2-NA = 6-ACETOXY-2-NAPHTHOIC ACID
4-Ac-3-OMe CA = 4-ACETOXY-3-METHOXYCINNAMIC ACID
N,O-DiAcTyr = N,O-DIACETOXY-L-TYROSINE
S. Aur = Staphylococcus aureus
P. Aer = Psuedomonas aeruginosa
C. Acu = Colletotrichum acutatum

Bisphenols (Z and Z′═H) prepared according to the aforementioned procedure, and biological data, are provided in Table 2.

TABLE 2

| | S. aure | P. aeru | C. acut. | Sector A | Amine | Sector B |
|---|---|---|---|---|---|---|
| 239 | NM | NM | 0.063 | ME-P-OH BN | 1,3-DIAMINOPROPANE | 4-AC-BA |
| 240 | NM | NM | 0.053 | ME-P-OH BN | 1,3-DIAMINOPROPANE | 3-AC-BA |
| 241 | NM | | 0.080 | ME-3-OH BN | 1,3-DIAMINOPROPANE | 4-AC-BA |
| 242 | NM | NM | 0.031 | ME-P-OH BN | 1,3-DIAMINOPROPANE | 4-AC-CA |
| 243 | 0.024 | 0.016 | 0.004 | ME-P-OH BN | 1,3-DIAMINOPROPANE | 6-AC-2-NA |
| 244 | NM | 0.063 | 0.043 | ME-P-OH BN | 2,2-DIMETHYL-1,3-PROPANEDIAMINE | 4-AC-BA |
| 245 | NM | NM | 0.039 | ME-P-OH BN | 2,2-DIMETHYL-1,3-PROPANEDIAMINE | 3-AC-BA |
| 246 | NM | | 0.006 | ME-3-OH BN | 2,2-DIMETHYL-1,3-PROPANEDIAMINE | 4-AC-BA |
| 247 | 0.034 | 0.06 | 0.007 | ME-P-OH BN | 2,2-DIMETHYL-1,3-PROPANEDIAMINE | 4-AC-CA |
| 248 | 0.036 | 0.045 | 0.005 | ME-P-OH BN | 2,2-DIMETHYL-1,3-PROPANEDIAMINE | 2-AC-CA |
| 249 | 0.024 | NM | 0.012 | ME-P-OH BN | 2,2-DIMETHYL-1,3-PROPANEDIAMINE | AC SA |
| 250 | <.017 | <.005 | <.005 | ME-P-OH BN | 2,2-DIMETHYL-1,3-PROPANEDIAMINE | 6-AC-2-NA |
| 251 | NM | NM | 0.068 | ME-P-OH BN | ETHYLENEDIAMINE | 4-AC-BA |
| 252 | NM | 0.046 | 0.071 | ME-P-OH BN | ETHYLENEDIAMINE | 3-AC-BA |
| 253 | NM | | 0.013 | ME-3-OH BN | ETHYLENEDIAMINE | 4-AC-BA |
| 254 | 0.368 | 0.259 | 0.018 | ME-P-OH BN | ETHYLENEDIAMINE | 4-AC-CA |
| 255 | 0.059 | NM | 0.021 | ME-P-OH BN | ETHYLENEDIAMINE | 2-AC-CA |
| 256 | NM | 0.081 | 0.041 | ME-P-OH BN | ETHYLENEDIAMINE | AC SA |
| 257 | 0.009 | 0.005 | 0.015 | ME-P-OH BN | ETHYLENEDIAMINE | 6-AC-2-NA |
| 258 | NM | NM | 0.090 | ME-P-OH BN | CIS-1,2-DIAMINOCYCLOHEXANE | 4-AC-BA |
| 259 | 0.092 | >.05 | 0.047 | ME-P-OH BN | CIS-1,2-DIAMINOCYCLOHEXANE | 3-AC-BA |
| 260 | 0.018 | RT | 0.008 | ME-P-OH BN | CIS-1,2-DIAMINOCYCLOHEXANE | 4-AC-CA |
| 261 | 0.011 | 0.019 | 0.013 | ME-P-OH BN | CIS-1,2-DIAMINOCYCLOHEXANE | 2-AC-CA |
| 262 | 0.042 | NM | NM | ME-P-OH BN | CIS-1,2-DIAMINOCYCLOHEXANE | AC SA |
| 263 | <.017 | <.005 | <.005 | ME-P-OH BN | CIS-1,2-DIAMINOCYCLOHEXANE | 6-AC-2-NA |
| 264 | NM | 0.033 | 0.066 | ME-P-OH BN | 1,4-DIAMINOBUTANE | 4-AC-BA |
| 265 | NM | NM | 0.052 | ME-P-OH BN | 1,4-DIAMINOBUTANE | 3-AC-BA |
| 266 | ND | | 0.020 | ME-3-OH BN | 1,4-DIAMINOBUTANE | 4-AC-BA |
| 267 | 0.112 | 0.082 | 0.021 | ME-P-OH BN | 1,4-DIAMINOBUTANE | 4-AC-CA |
| 268 | 0.048 | 3.205 | 0.011 | ME-P-OH BN | 1,4-DIAMINOBUTANE | 2-AC-CA |
| 269 | NM | 0.057 | 0.071 | ME-P-OH BN | 1,4-DIAMINOBUTANE | AC SA |
| 270 | 0.006 | 0.003 | 0.006 | ME-P-OH BN | 1,4-DIAMINOBUTANE | 6-AC-2-NA |
| 271 | 0.055 | 0.079 | 0.017 | ME-P-OH BN | TRANS-1,4-DIAMINOCYCLOHEXANE | 4-AC-BA |
| 272 | 0.020 | RT | 0.009 | ME-P-OH BN | TRANS-1,4-DIAMINOCYCLOHEXANE | 3-AC-BA |
| 273 | ND | | 0.001 | ME-3-OH BN | TRANS-1,4-DIAMINOCYCLOHEXANE | 4-AC-BA |
| 274 | 0.024 | RT | 0.010 | ME-P-OH BN | TRANS-1,4-DIAMINOCYCLOHEXANE | 4-AC-CA |
| 275 | 0.073 | RT | <.005 | ME-P-OH BN | TRANS-1,4-DIAMINOCYCLOHEXANE | 2-AC-CA |
| 276 | 0.041 | RT | 0.031 | ME-P-OH BN | TRANS-1,4-DIAMINOCYCLOHEXANE | AC SA |
| 277 | <.017 | 0.001 | 0.003 | ME-P-OH BN | TRANS-1,4-DIAMINOCYCLOHEXANE | 6-AC-2-NA |
| 278 | 0.083 | NM | 0.028 | ME-P-OH BN | 1,5-DIAMINOPENTANE | 4-AC-BA |
| 279 | 0.045 | NM | 0.026 | ME-P-OH BN | 1,5-DIAMINOPENTANE | 3-AC-BA |
| 280 | ND | | 0.056 | ME-3-OH BN | 1,5-DIAMINOPENTANE | 4-AC-BA |
| 281 | 0.368 | >.05 | 0.006 | ME-P-OH BN | 1,5-DIAMINOPENTANE | 4-AC-CA |
| 282 | 0.060 | NM | 0.006 | ME-P-OH BN | 1,5-DIAMINOPENTANE | 2-AC-CA |
| 283 | 0.106 | 0.072 | 0.008 | ME-P-OH BN | 1,5-DIAMINOPENTANE | AC SA |
| 284 | 0.011 | 0.005 | 0.001 | ME-P-OH BN | 1,5-DIAMINOPENTANE | 6-AC-2-NA |
| 285 | NM | 0.043 | >.05 | ME-P-OH BN | 2,2′-THIOBIS(ETHYLAMINE) | 4-AC-BA |
| 286 | NM | NM | NM | ME-P-OH BN | 2,2′-THIOBIS(ETHYLAMINE) | 3-AC-BA |
| 287 | ND | | 0.004 | ME-3-OH BN | 2,2′-THIOBIS(ETHYLAMINE) | 4-AC-BA |
| 288 | NM | NM | 0.048 | ME-P-OH BN | 2,2′-THIOBIS(ETHYLAMINE) | 4-AC-CA |
| 289 | NM | NM | >.05 | ME-P-OH BN | 2,2′-THIOBIS(ETHYLAMINE) | 2-AC-CA |
| 290 | NM | 0.069 | 0.033 | ME-P-OH BN | 2,2′-THIOBIS(ETHYLAMINE) | AC SA |
| 291 | <.017 | 0.024 | <.005 | ME-P-OH BN | 2,2′-THIOBIS(ETHYLAMINE) | 6-AC-2-NA |
| 292 | 0.032 | 0.069 | 0.007 | ME-P-OH BN | N,N-BIS(3-AMINOPROPYL)METHYLAMINE | 4-AC-BA |
| 293 | 0.047 | 0.133 | 0.008 | ME-P-OH BN | N,N-BIS(3-AMINOPROPYL)METHYLAMINE | 3-AC-BA |
| 294 | ND | | 0.003 | ME-3-OH BN | N,N-BIS(3-AMINOPROPYL)METHYLAMINE | 4-AC-BA |
| 295 | 0.028 | RT | 0.008 | ME-P-OH BN | N,N-BIS(3-AMINOPROPYL)METHYLAMINE | 4-AC-CA |
| 296 | 0.023 | 0.022 | 0.002 | ME-P-OH BN | N,N-BIS(3-AMINOPROPYL)METHYLAMINE | 2-AC-CA |
| 297 | 0.019 | 0.058 | 0.011 | ME-P-OH BN | N,N-BIS(3-AMINOPROPYL)METHYLAMINE | AC SA |
| 298 | <.017 | 0.001 | <.005 | ME-P-OH BN | N,N-BIS(3-AMINOPROPYL)METHYLAMINE | 6-AC-2-NA |
| 299 | NM | 0.071 | 0.044 | ME-P-OH BN | HOMOPIPERAZINE | 4-AC-BA |
| 300 | NM | 0.125 | 0.038 | ME-P-OH BN | HOMOPIPERAZINE | 3-AC-BA |
| 301 | ND | | 0.015 | ME-3-OH BN | HOMOPIPERAZINE | 4-AC-BA |
| 302 | 0.029 | 0.084 | 0.009 | ME-P-OH BN | HOMOPIPERAZINE | 4-AC-CA |
| 303 | 0.033 | 0.028 | 0.004 | ME-P-OH BN | HOMOPIPERAZINE | 2-AC-CA |
| 304 | NM | 0.125 | 0.026 | ME-P-OH BN | HOMOPIPERAZINE | AC SA |
| 305 | <.017 | <.005 | <.005 | ME-P-OH BN | HOMOPIPERAZINE | 6-AC-2-NA |
| 306 | 0.030 | 0.052 | 0.014 | ME-P-OH BN | 1,6-HEXANEDIAMINE | 4-AC-BA |
| 307 | 0.042 | NM | 0.024 | ME-P-OH BN | 1,6-HEXANEDIAMINE | 3-AC-BA |
| 308 | ND | | 0.001 | ME-3-OH BN | 1,6-HEXANEDIAMINE | 4-AC-BA |
| 309 | 0.035 | 0.064 | <.005 | ME-P-OH BN | 1,6-HEXANEDIAMINE | 4-AC-CA |
| 310 | 0.085 | 0.066 | <.005 | ME-P-OH BN | 1,6-HEXANEDIAMINE | 2-AC-CA |
| 311 | NM | 0.041 | 0.012 | ME-P-OH BN | 1,6-HEXANEDIAMINE | AC SA |
| 312 | 0.010 | 0.006 | <.005 | ME-P-OH BN | 1,6-HEXANEDIAMINE | 6-AC-2-NA |
| 313 | NM | NM | >.05 | ME-P-OH BN | N,N′-DIMETHYLETHYLENEDIAMINE | 4-AC-BA |
| 314 | NM | NM | 0.028 | ME-P-OH BN | N,N′-DIMETHYLETHYLENEDIAMINE | 3-AC-BA |
| 315 | ND | | 0.055 | ME-3-OH BN | N,N′-DIMETHYLETHYLENEDIAMINE | 4-AC-BA |
| 316 | 0.018 | NM | 0.010 | ME-P-OH BN | N,N′-DIMETHYLETHYLENEDIAMINE | 4-AC-CA |
| 317 | <.017 | 0.049 | 0.009 | ME-P-OH BN | N,N′-DIMETHYLETHYLENEDIAMINE | 2-AC-CA |
| 318 | 0.032 | NM | 0.056 | ME-P-OH BN | N,N′-DIMETHYLETHYLENEDIAMINE | AC SA |
| 319 | <.017 | 0.009 | <.005 | ME-P-OH BN | N,N′-DIMETHYLETHYLENEDIAMINE | 6-AC-2-NA |
| 320 | 0.031 | NM | 0.048 | ME-P-OH BN | N,N′-DIETHYL-1,3-PROPANEDIAMINE | 4-AC-BA |
| 321 | 0.042 | NM | 0.039 | ME-P-OH BN | N,N′-DIETHYL-1,3-PROPANEDIAMINE | 3-AC-BA |
| 322 | ND | | 0.014 | ME-3-OH BN | N,N′-DIETHYL-1,3-PROPANEDIAMINE | 4-AC-BA |
| 323 | <.017 | NM | 0.007 | ME-P-OH BN | N,N′-DIETHYL-1,3-PROPANEDIAMINE | 4-AC-CA |
| 324 | <.017 | NM | 0.007 | ME-P-OH BN | N,N′-DIETHYL-1,3-PROPANEDIAMINE | 2-AC-CA |
| 325 | 0.036 | NM | 0.018 | ME-P-OH BN | N,N′-DIETHYL-1,3-PROPANEDIAMINE | AC SA |
| 326 | <.017 | 0.002 | <.005 | ME-P-OH BN | N,N′-DIETHYL-1,3-PROPANEDIAMINE | 6-AC-2-NA |
| 327 | 0.092 | NM | 0.032 | ME-P-OH BN | 1,8-DIAMINO-3,6-DIOXAOCTANE | 4-AC-BA |
| 328 | 0.097 | NM | 0.039 | ME-P-OH BN | 1,8-DIAMINO-3,6-DIOXAOCTANE | 3-AC-BA |
| 329 | ND | | 0.008 | ME-3-OH BN | 1,8-DIAMINO-3,6-DIOXAOCTANE | 4-AC-BA |
| 330 | 0.033 | NM | >.05 | ME-P-OH BN | 1,8-DIAMINO-3,6-DIOXAOCTANE | 4-AC-CA |
| 331 | 0.074 | NM | 0.006 | ME-P-OH BN | 1,8-DIAMINO-3,6-DIOXAOCTANE | 2-AC-CA |
| 332 | 0.031 | NM | 0.031 | ME-P-OH BN | 1,8-DIAMINO-3,6-DIOXAOCTANE | AC SA |
| 333 | <.017 | 0.073 | 0.038 | ME-P-OH BN | 1,8-DIAMINO-3,6-DIOXAOCTANE | 6-AC-2-NA |
| 334 | 0.076 | NM | 0.031 | ME-P-OH BN | N,N′-DIETHYL-2-BUTENE-1,4-DIAMINE | 4-AC-BA |
| 335 | 0.028 | NM | NM | ME-P-OH BN | N,N′-DIETHYL-2-BUTENE-1,4-DIAMINE | 3-AC-BA |
| 336 | ND | | 0.016 | ME-3-OH BN | N,N′-DIETHYL-2-BUTENE-1,4-DIAMINE | 4-AC-BA |
| 337 | <.017 | NM | 0.008 | ME-P-OH BN | N,N′-DIETHYL-2-BUTENE-1,4-DIAMINE | 4-AC-CA |
| 338 | <.017 | NM | 0.009 | ME-P-OH BN | N,N′-DIETHYL-2-BUTENE-1,4-DIAMINE | 2-AC-CA |
| 339 | 0.032 | NM | 0.041 | ME-P-OH BN | N,N′-DIETHYL-2-BUTENE-1,4-DIAMINE | AC SA |
| 340 | <.017 | 0.007 | <.005 | ME-P-OH BN | N,N′-DIETHYL-2-BUTENE-1,4-DIAMINE | 6-AC-2-NA |
| 341 | NM | NM | 0.153 | ME-3-CL-4-OH BN | 1,3-DIAMINOPROPANE | 4-AC-BA |
| 342 | 0.036 | NM | 0.040 | ME-3-CL-4-OH BN | 1,3-DIAMINOPROPANE | 3-AC-BA |
| 343 | 0.029 | NM | 0.026 | ME-3-CL-4-OH BN | 1,3-DIAMINOPROPANE | 4-AC-CA |
| 344 | 0.033 | NM | 0.012 | ME-3-CL-4-OH BN | 1,3-DIAMINOPROPANE | 2-AC-CA |
| 345 | 0.031 | NM | 0.021 | ME-3-CL-4-OH BN | 1,3-DIAMINOPROPANE | AC SA |
| 346 | 0.015 | 0.03 | <.005 | ME-3-CL-4-OH BN | 1,3-DIAMINOPROPANE | 6-AC-2-NA |
| 347 | <.017 | 0.12 | 0.023 | ME-3-CL-4-OH BN | 2,2-DIMETHYL-1,3-PROPANEDIAMINE | 4-AC-BA |
| 348 | 0.008 | 0.058 | 0.007 | ME-3-CL-4-OH BN | 2,2-DIMETHYL-1,3-PROPANEDIAMINE | 3-AC-BA |
| 349 | 0.028 | NM | 0.012 | ME-3-CL-4-OH BN | 2,2-DIMETHYL-1,3-PROPANEDIAMINE | 4-AC-CA |
| 350 | 0.006 | 0.044 | 0.011 | ME-3-CL-4-OH BN | 2,2-DIMETHYL-1,3-PROPANEDIAMINE | 2-AC-CA |
| 351 | <.017 | NM | 0.016 | ME-3-CL-4-OH BN | 2,2-DIMETHYL-1,3-PROPANEDIAMINE | AC SA |
| 352 | <.017 | 0.007 | <.005 | ME-3-CL-4-OH BN | 2,2-DIMETHYL-1,3-PROPANEDIAMINE | 6-AC-2-NA |
| 353 | NM | NM | 0.035 | ME-3-CL-4-OH BN | ETHYLENEDIAMINE | 4-AC-BA |
| 354 | NM | NM | 0.038 | ME-3-CL-4-OH BN | ETHYLENEDIAMINE | 3-AC-BA |
| 355 | 0.030 | NM | 0.025 | ME-3-CL-4-OH BN | ETHYLENEDIAMINE | 4-AC-CA |
| 356 | 0.034 | NM | 0.016 | ME-3-CL-4-OH BN | ETHYLENEDIAMINE | 2-AC-CA |
| 357 | NM | NM | 0.037 | ME-3-CL-4-OH BN | ETHYLENEDIAMINE | AC SA |
| 358 | <.017 | 0.029 | 0.001 | ME-3-CL-4-OH BN | ETHYLENEDIAMINE | 6-AC-2-NA |
| 359 | 0.011 | NM | <.005 | ME-3-CL-4-OH BN | CIS-1,2-DIAMINOCYCLOHEXANE | 4-AC-BA |
| 360 | 0.015 | 0.065 | <.005 | ME-3-CL-4-OH BN | CIS-1,2-DIAMINOCYCLOHEXANE | 3-AC-BA |
| 361 | 0.023 | 0.082 | <.005 | ME-3-CL-4-OH BN | CIS-1,2-DIAMINOCYCLOHEXANE | 4-AC-CA |
| 362 | 0.027 | 0.116 | 0.013 | ME-3-CL-4-OH BN | CIS-1,2-DIAMINOCYCLOHEXANE | 2-AC-CA |
| 363 | 0.013 | 0.082 | 0.022 | ME-3-CL-4-OH BN | CIS-1,2-DIAMINOCYCLOHEXANE | AC SA |
| 364 | 0.004 | 0.005 | <.005 | ME-3-CL-4-OH BN | CIS-1,2-DIAMINOCYCLOHEXANE | 6-AC-2-NA |
| 365 | 0.270 | NM | 0.026 | ME-3-CL-4-OH BN | 1,4-DIAMINOBUTANE | 4-AC-BA |
| 366 | NM | NM | 0.040 | ME-3-CL-4-OH BN | 1,4-DIAMINOBUTANE | 3-AC-BA |
| 367 | 0.097 | 0.056 | 0.014 | ME-3-CL-4-OH BN | 1,4-DIAMINOBUTANE | 4-AC-CA |
| 368 | NM | 0.084 | 0.025 | ME-3-CL-4-OH BN | 1,4-DIAMINOBUTANE | 2-AC-CA |
| 369 | 0.231 | 0.101 | 0.029 | ME-3-CL-4-OH BN | 1,4-DIAMINOBUTANE | AC SA |
| 370 | 0.015 | 0.018 | <.005 | ME-3-CL-4-OH BN | 1,4-DIAMINOBUTANE | 6-AC-2-NA |
| 371 | <.017 | RT | 0.005 | ME-3-CL-4-OH BN | TRANS-1,4-DIAMINOCYCLOHEXANE | 4-AC-BA |
| 372 | 0.005 | RT | 0.004 | ME-3-CL-4-OH BN | TRANS-1,4-DIAMINOCYCLOHEXANE | 3-AC-BA |
| 373 | 0.038 | RT | 0.006 | ME-3-CL-4-OH BN | TRANS-1,4-DIAMINOCYCLOHEXANE | 4-AC-CA |
| 374 | 0.036 | RT | <.005 | ME-3-CL-4-OH BN | TRANS-1,4-DIAMINOCYCLOHEXANE | 2-AC-CA |
| 375 | 0.043 | 0.049 | 0.011 | ME-3-CL-4-OH BN | TRANS-1,4-DIAMINOCYCLOHEXANE | AC SA |
| 376 | 0.005 | 0.005 | <.005 | ME-3-CL-4-OH BN | TRANS-1,4-DIAMINOCYCLOHEXANE | 6-AC-2-NA |
| 377 | NM | 0.108 | 0.013 | ME-3-CL-4-OH BN | 1,5-DIAMINOPENTANE | 4-AC-BA |
| 378 | NM | 0.067 | 0.026 | ME-3-CL-4-OH BN | 1,5-DIAMINOPENTANE | 3-AC-BA |
| 379 | 0.036 | 0.058 | 0.000 | ME-3-CL-4-OH BN | 1,5-DIAMINOPENTANE | 4-AC-CA |
| 380 | 0.033 | 0.097 | 0.002 | ME-3-CL-4-OH BN | 1,5-DIAMINOPENTANE | 2-AC-CA |
| 381 | NM | 0.084 | 0.044 | ME-3-CL-4-OH BN | 1,5-DIAMINOPENTANE | AC SA |
| 382 | 0.010 | 0.005 | <.005 | ME-3-CL-4-OH BN | 1,5-DIAMINOPENTANE | 6-AC-2-NA |
| 383 | 0.063 | 0.505 | 0.020 | ME-3-CL-4-OH BN | 2,2′-THIOBIS(ETHYLAMINE) | 4-AC-BA |
| 384 | 0.142 | NM | 0.010 | ME-3-CL-4-OH BN | 2,2′-THIOBIS(ETHYLAMINE) | 3-AC-BA |
| 385 | 0.022 | 0.1 | 0.007 | ME-3-CL-4-OH BN | 2,2′-THIOBIS(ETHYLAMINE) | 4-AC-CA |
| 386 | 0.025 | 0.129 | <.005 | ME-3-CL-4-OH BN | 2,2′-THIOBIS(ETHYLAMINE) | 2-AC-CA |
| 387 | 0.034 | 0.061 | 0.082 | ME-3-CL-4-OH BN | 2,2′-THIOBIS(ETHYLAMINE) | AC SA |
| 388 | 0.009 | 0.011 | <.005 | ME-3-CL-4-OH BN | 2,2′-THIOBIS(ETHYLAMINE) | 6-AC-2-NA |
| 389 | 0.059 | 0.158 | NM | ME-3-CL-4-OH BN | N,N-BIS(3-AMINOPROPYL)METHYLAMINE | 4-AC-BA |
| 390 | 0.020 | 0.023 | 0.005 | ME-3-CL-4-OH BN | N,N-BIS(3-AMINOPROPYL)METHYLAMINE | 3-AC-BA |
| 391 | 0.016 | 0.012 | 0.008 | ME-3-CL-4-OH BN | N,N-BIS(3-AMINOPROPYL)METHYLAMINE | 4-AC-CA |
| 392 | 0.010 | 0.017 | 0.005 | ME-3-CL-4-OH BN | N,N-BIS(3-AMINOPROPYL)METHYLAMINE | 2-AC-CA |
| 393 | 0.012 | 0.033 | 0.000 | ME-3-CL-4-OH BN | N,N-BIS(3-AMINOPROPYL)METHYLAMINE | AC SA |
| 394 | 0.003 | <.005 | 0.002 | ME-3-CL-4-OH BN | N,N-BIS(3-AMINOPROPYL)METHYLAMINE | 6-AC-2-NA |
| 395 | NM | 0.066 | 0.033 | ME-3-CL-4-OH BN | HOMOPIPERAZINE | 4-AC-BA |
| 396 | NM | 0.067 | 0.029 | ME-3-CL-4-OH BN | HOMOPIPERAZINE | 3-AC-BA |
| 397 | 0.118 | 0.05 | 0.016 | ME-3-CL-4-OH BN | HOMOPIPERAZINE | 4-AC-CA |
| 398 | 0.014 | 0.012 | 0.002 | ME-3-CL-4-OH BN | HOMOPIPERAZINE | 2-AC-CA |
| 399 | NM | NM | 0.021 | ME-3-CL-4-OH BN | HOMOPIPERAZINE | AC SA |
| 400 | 0.001 | 0.005 | <.005 | ME-3-CL-4-OH BN | HOMOPIPERAZINE | 6-AC-2-NA |
| 401 | 0.033 | 0.052 | 0.007 | ME-3-CL-4-OH BN | 1,6-HEXANEDIAMINE | 4-AC-BA |
| 402 | 0.037 | 0.044 | 0.006 | ME-3-CL-4-OH BN | 1,6-HEXANEDIAMINE | 3-AC-BA |
| 403 | 0.033 | 0.101 | 0.012 | ME-3-CL-4-OH BN | 1,6-HEXANEDIAMINE | 4-AC-CA |
| 404 | 0.008 | 0.142 | <.005 | ME-3-CL-4-OH BN | 1,6-HEXANEDIAMINE | 2-AC-CA |
| 405 | 0.052 | 0.032 | 0.009 | ME-3-CL-4-OH BN | 1,6-HEXANEDIAMINE | AC SA |
| 406 | 0.014 | 0.012 | 0.003 | ME-3-CL-4-OH BN | 1,6-HEXANEDIAMINE | 6-AC-2-NA |
| 407 | NM | RT | 0.011 | ME-3-CL-4-OH BN | N,N′-DIMETHYLETHYLENEDIAMINE | 4-AC-BA |
| 408 | NM | NM | >.05 | ME-3-CL-4-OH BN | N,N′-DIMETHYLETHYLENEDIAMINE | 3-AC-BA |
| 409 | 0.033 | 0.062 | 0.017 | ME-3-CL-4-OH BN | N,N′-DIMETHYLETHYLENEDIAMINE | 4-AC-CA |
| 410 | 0.034 | 0.04 | 0.007 | ME-3-CL-4-OH BN | N,N′-DIMETHYLETHYLENEDIAMINE | 2-AC-CA |
| 411 | NM | 0.189 | 0.019 | ME-3-CL-4-OH BN | N,N′-DIMETHYLETHYLENEDIAMINE | AC SA |
| 412 | 0.002 | <.005 | 0.002 | ME-3-CL-4-OH BN | N,N′-DIMETHYLETHYLENEDIAMINE | 6-AC-2-NA |
| 413 | | NM | 0.061 | ME-3-CL-4-OH BN | N,N′-DIETHYL-1,3-PROPANEDIAMINE | 4-AC-BA |
| 414 | | NM | 0.053 | ME-3-CL-4-OH BN | N,N′-DIETHYL-1,3-PROPANEDIAMINE | 3-AC-BA |
| 415 | | >.05 | 0.009 | ME-3-CL-4-OH BN | N,N′-DIETHYL-1,3-PROPANEDIAMINE | 4-AC-CA |
| 416 | 0.010 | 0.025 | 0.007 | ME-3-CL-4-OH BN | N,N′-DIETHYL-1,3-PROPANEDIAMINE | 2-AC-CA |
| 417 | 0.905 | NM | 0.036 | ME-3-CL-4-OH BN | N,N′-DIETHYL-1,3-PROPANEDIAMINE | AC SA |
| 418 | 0.010 | 0.007 | 0.001 | ME-3-CL-4-OH BN | N,N′-DIETHYL-1,3-PROPANEDIAMINE | 6-AC-2-NA |
| 419 | NM | NM | 0.015 | ME-3-CL-4-OH BN | 1,8-DIAMINO-3,6-DIOXAOCTANE | 4-AC-BA |
| 420 | NM | NM | 0.027 | ME-3-CL-4-OH BN | 1,8-DIAMINO-3,6-DIOXAOCTANE | 3-AC-BA |
| 421 | 0.151 | NM | 0.014 | ME-3-CL-4-OH BN | 1,8-DIAMINO-3,6-DIOXAOCTANE | 4-AC-CA |
| 422 | 0.029 | NM | 0.013 | ME-3-CL-4-OH BN | 1,8-DIAMINO-3,6-DIOXAOCTANE | 2-AC-CA |
| 423 | 0.090 | NM | 0.017 | ME-3-CL-4-OH BN | 1,8-DIAMINO-3,6-DIOXAOCTANE | AC SA |
| 424 | <.005 | 0.011 | 0.002 | ME-3-CL-4-OH BN | 1,8-DIAMINO-3,6-DIOXAOCTANE | 6-AC-2-NA |
| 425 | 0.105 | NM | 0.008 | ME-3-CL-4-OH BN | N,N′-DIETHYL-2-BUTENE-1,4-DIAMINE | 4-AC-BA |
| 426 | 0.046 | NM | 0.006 | ME-3-CL-4-OH BN | N,N′-DIETHYL-2-BUTENE-1,4-DIAMINE | 3-AC-BA |
| 427 | 0.008 | 0.028 | 0.006 | ME-3-CL-4-OH BN | N,N′-DIETHYL-2-BUTENE-1,4-DIAMINE | 4-AC-CA |
| 428 | 0.010 | 0.02 | 0.004 | ME-3-CL-4-OH BN | N,N′-DIETHYL-2-BUTENE-1,4-DIAMINE | 2-AC-CA |
| 429 | 0.040 | 0.073 | 0.015 | ME-3-CL-4-OH BN | N,N′-DIETHYL-2-BUTENE-1,4-DIAMINE | AC SA |
| 430 | <.005 | 0.026 | 0.001 | ME-3-CL-4-OH BN | N,N′-DIETHYL-2-BUTENE-1,4-DIAMINE | 6-AC-2-NA |
| 431 | NM | RT | 0.025 | ME-3-OH BN | 1,3-DIAMINOPROPANE | 3-AC-BA |
| 432 | NM | 0.228 | 0.021 | ME-3-OH BN | 1,3-DIAMINOPROPANE | 4-AC-CA |
| 433 | NM | >.05 | 0.021 | ME-3-OH BN | 1,3-DIAMINOPROPANE | 2-AC-CA |
| 434 | NM | NM | 0.001 | ME-3-OH BN | 1,3-DIAMINOPROPANE | AC SA |
| 435 | 0.032 | 0.021 | 0.240 | ME-3-OH BN | 1,3-DIAMINOPROPANE | 6-AC-2-NA |
| 436 | NM | 0.062 | 0.007 | ME-3-OH BN | 2,2-DIMETHYL-1,3-PROPANEDIAMINE | 3-AC-BA |
| 437 | 0.122 | 0.045 | 0.014 | ME-3-OH BN | 2,2-DIMETHYL-1,3-PROPANEDIAMINE | 4-AC-CA |
| 438 | 0.435 | 0.056 | 0.002 | ME-3-OH BN | 2,2-DIMETHYL-1,3-PROPANEDIAMINE | 2-AC-CA |
| 439 | NM | 0.061 | 0.075 | ME-3-OH BN | 2,2-DIMETHYL-1,3-PROPANEDIAMINE | AC SA |
| 440 | 0.010 | 0.032 | 0.010 | ME-3-OH BN | 2,2-DIMETHYL-1,3-PROPANEDIAMINE | 6-AC-2-NA |
| 441 | NM | >.05 | 0.135 | ME-3-OH BN | ETHYLENEDIAMINE | 3-AC-BA |
| 442 | NM | >.05 | 0.005 | ME-3-OH BN | ETHYLENEDIAMINE | 4-AC-CA |
| 443 | NM | >.05 | 0.407 | ME-3-OH BN | ETHYLENEDIAMINE | 2-AC-CA |
| 444 | NM | >.05 | NM | ME-3-OH BN | ETHYLENEDIAMINE | AC SA |
| 445 | 0.026 | 0.019 | 0.007 | ME-3-OH BN | ETHYLENEDIAMINE | 6-AC-2-NA |
| 446 | NM | NM | 0.013 | ME-3-OH BN | CIS-1,2-DIAMINOCYCLOHEXANE | 4-AC-BA |
| 447 | NM | NM | 0.038 | ME-3-OH BN | CIS-1,2-DIAMINOCYCLOHEXANE | 3-AC-BA |
| 448 | 0.030 | 0.03 | 0.001 | ME-3-OH BN | CIS-1,2-DIAMINOCYCLOHEXANE | 4-AC-CA |
| 449 | 0.043 | 0.031 | NM | ME-3-OH BN | CIS-1,2-DIAMINOCYCLOHEXANE | 2-AC-CA |
| 450 | NM | 0.027 | 0.014 | ME-3-OH BN | CIS-1,2-DIAMINOCYCLOHEXANE | AC SA |
| 451 | <.005 | 0.001 | 0.006 | ME-3-OH BN | CIS-1,2-DIAMINOCYCLOHEXANE | 6-AC-2-NA |
| 452 | 0.199 | <.005 | 0.001 | ME-3-OH BN | 1,4-DIAMINOBUTANE | 3-AC-BA |
| 453 | 0.148 | NM | 0.024 | ME-3-OH BN | 1,4-DIAMINOBUTANE | 4-AC-CA |
| 454 | 0.301 | NM | 0.005 | ME-3-OH BN | 1,4-DIAMINOBUTANE | 2-AC-CA |
| 455 | NM | NM | 0.006 | ME-3-OH BN | 1,4-DIAMINOBUTANE | AC SA |
| 456 | 0.013 | 0.011 | 0.010 | ME-3-OH BN | 1,4-DIAMINOBUTANE | 6-AC-2-NA |
| 457 | 0.063 | >.05 | 0.061 | ME-3-OH BN | TRANS-1,4-DIAMINOCYCLOHEXANE | 3-AC-BA |
| 458 | 0.031 | 0.021 | 0.028 | ME-3-OH BN | TRANS-1,4-DIAMINOCYCLOHEXANE | 4-AC-CA |
| 459 | 0.039 | 0.024 | 0.007 | ME-3-OH BN | TRANS-1,4-DIAMINOCYCLOHEXANE | 2-AC-CA |
| 460 | 0.059 | 0.408 | 0.027 | ME-3-OH BN | TRANS-1,4-DIAMINOCYCLOHEXANE | AC SA |
| 461 | 0.024 | 0.005 | 0.002 | ME-3-OH BN | TRANS-1,4-DIAMINOCYCLOHEXANE | 6-AC-2-NA |
| 462 | NM | NM | 0.005 | ME-3-OH BN | 1,5-DIAMINOPENTANE | 3-AC-BA |
| 463 | NM | NM | 0.010 | ME-3-OH BN | 1,5-DIAMINOPENTANE | 4-AC-CA |
| 464 | 0.084 | >.05 | 0.048 | ME-3-OH BN | 1,5-DIAMINOPENTANE | 2-AC-CA |
| 465 | NM | NM | 0.001 | ME-3-OH BN | 1,5-DIAMINOPENTANE | AC SA |
| 466 | 0.022 | 0.012 | 0.040 | ME-3-OH BN | 1,5-DIAMINOPENTANE | 6-AC-2-NA |
| 467 | 0.397 | >.05 | NM | ME-3-OH BN | 2,2′-THIOBIS(ETHYLAMINE) | 3-AC-BA |
| 468 | 0.199 | 0.054 | 0.007 | ME-3-OH BN | 2,2′-THIOBIS(ETHYLAMINE) | 4-AC-CA |
| 469 | 0.232 | 0.087 | 0.004 | ME-3-OH BN | 2,2′-THIOBIS(ETHYLAMINE) | 2-AC-CA |
| 470 | NM | 0.247 | 0.026 | ME-3-OH BN | 2,2′-THIOBIS(ETHYLAMINE) | AC SA |
| 471 | 0.009 | 0.016 | <.005 | ME-3-OH BN | 2,2′-THIOBIS(ETHYLAMINE) | 6-AC-2-NA |
| 472 | 0.069 | 0.185 | 0.021 | ME-3-OH BN | N,N-BIS(3-AMINOPROPYL)METHYLAMINE | 3-AC-BA |
| 473 | 0.022 | 0.03 | 0.000 | ME-3-OH BN | N,N-BIS(3-AMINOPROPYL)METHYLAMINE | 4-AC-CA |
| 474 | 0.030 | 0.062 | 0.021 | ME-3-OH BN | N,N-BIS(3-AMINOPROPYL)METHYLAMINE | 2-AC-CA |
| 475 | 0.041 | 0.016 | 0.010 | ME-3-OH BN | N,N-BIS(3-AMINOPROPYL)METHYLAMINE | AC SA |
| 476 | 0.007 | 0.001 | 0.019 | ME-3-OH BN | N,N-BIS(3-AMINOPROPYL)METHYLAMINE | 6-AC-2-NA |
| 477 | 0.368 | NM | 0.003 | ME-3-OH BN | HOMOPIPERAZINE | 3-AC-BA |
| 478 | 0.037 | 0.079 | 0.026 | ME-3-OH BN | HOMOPIPERAZINE | 4-AC-CA |
| 479 | 0.017 | 0.043 | 0.012 | ME-3-OH BN | HOMOPIPERAZINE | 2-AC-CA |
| 480 | NM | >.05 | 0.009 | ME-3-OH BN | HOMOPIPERAZINE | AC SA |
| 481 | <.005 | <.005 | 0.051 | ME-3-OH BN | HOMOPIPERAZINE | 6-AC-2-NA |
| 482 | NM | >.05 | 0.050 | ME-3-OH BN | 1,6-HEXANEDIAMINE | 3-AC-BA |
| 483 | 0.127 | NM | 0.009 | ME-3-OH BN | 1,6-HEXANEDIAMINE | 4-AC-CA |
| 484 | 0.046 | NM | 0.014 | ME-3-OH BN | 1,6-HEXANEDIAMINE | 2-AC-CA |
| 485 | 0.035 | >.05 | 0.015 | ME-3-OH BN | 1,6-HEXANEDIAMINE | AC SA |
| 486 | 0.001 | 0.009 | 0.003 | ME-3-OH BN | 1,6-HEXANEDIAMINE | 6-AC-2-NA |
| 487 | 0.072 | 0.173 | 0.033 | ME-3-OH BN | N,N′-DIMETHYLETHYLENEDIAMINE | 3-AC-BA |
| 488 | NM | NM | 0.010 | ME-3-OH BN | N,N′-DIMETHYLETHYLENEDIAMINE | 4-AC-CA |
| 489 | 0.040 | NM | 0.111 | ME-3-OH BN | N,N′-DIMETHYLETHYLENEDIAMINE | 2-AC-CA |
| 490 | NM | NM | 0.002 | ME-3-OH BN | N,N′-DIMETHYLETHYLENEDIAMINE | AC SA |
| 491 | 0.002 | 0.008 | 0.036 | ME-3-OH BN | N,N′-DIMETHYLETHYLENEDIAMINE | 6-AC-2-NA |
| 492 | 0.223 | NM | 0.033 | ME-3-OH BN | N,N′-DIETHYL-1,3-PROPANEDIAMINE | 3-AC-BA |
| 493 | 0.026 | NM | 0.368 | ME-3-OH BN | N,N′-DIETHYL-1,3-PROPANEDIAMINE | 4-AC-CA |
| 494 | 0.050 | NM | 0.001 | ME-3-OH BN | N,N′-DIETHYL-1,3-PROPANEDIAMINE | 2-AC-CA |
| 495 | NM | NM | 0.010 | ME-3-OH BN | N,N′-DIETHYL-1,3-PROPANEDIAMINE | AC SA |
| 496 | <.005 | 0.002 | 0.012 | ME-3-OH BN | N,N′-DIETHYL-1,3-PROPANEDIAMINE | 6-AC-2-NA |
| 497 | 0.107 | NM | 0.012 | ME-3-OH BN | 1,8-DIAMINO-3,6-DIOXAOCTANE | 3-AC-BA |
| 498 | 0.069 | NM | 0.038 | ME-3-OH BN | 1,8-DIAMINO-3,6-DIOXAOCTANE | 4-AC-CA |
| 499 | NM | NM | 0.024 | ME-3-OH BN | 1,8-DIAMINO-3,6-DIOXAOCTANE | 2-AC-CA |
| 500 | 0.143 | NM | 0.012 | ME-3-OH BN | 1,8-DIAMINO-3,6-DIOXAOCTANE | AC SA |
| 501 | <.005 | 0.026 | 0.018 | ME-3-OH BN | 1,8-DIAMINO-3,6-DIOXAOCTANE | 6-AC-2-NA |
| 502 | 0.135 | NM | 0.009 | ME-3-OH BN | N,N′-DIETHYL-2-BUTENE-1,4-DIAMINE | 3-AC-BA |
| 503 | 0.037 | 0.054 | 0.016 | ME-3-OH BN | N,N′-DIETHYL-2-BUTENE-1,4-DIAMINE | 4-AC-CA |
| 504 | 0.021 | NM | 0.000 | ME-3-OH BN | N,N′-DIETHYL-2-BUTENE-1,4-DIAMINE | 2-AC-CA |
| 505 | NM | NM | 0.020 | ME-3-OH BN | N,N′-DIETHYL-2-BUTENE-1,4-DIAMINE | AC SA |
| 506 | 0.005 | 0.004 | 0.021 | ME-3-OH BN | N,N′-DIETHYL-2-BUTENE-1,4-DIAMINE | 6-AC-2-NA |
| 507 | 0.062 | 0.055 | 0.016 | ME VAN | 1,3-DIAMINOPROPANE | 4-AC-BA |
| 508 | 0.067 | 0.093 | 0.007 | ME VAN | 1,3-DIAMINOPROPANE | 3-AC-BA |
| 509 | 0.061 | 0.055 | 0.021 | ME VAN | 1,3-DIAMINOPROPANE | 4-AC-CA |
| 510 | 0.135 | 0.017 | 0.001 | ME VAN | 1,3-DIAMINOPROPANE | 2-AC-CA |
| 511 | NM | NM | 0.050 | ME VAN | 1,3-DIAMINOPROPANE | AC SA |
| 512 | 0.010 | 0.013 | 0.007 | ME VAN | 1,3-DIAMINOPROPANE | 6-AC-2-NA |
| 513 | 0.074 | >.05 | 0.008 | ME VAN | 2,2-DIMETHYL-1,3-PROPANEDIAMINE | 4-AC-BA |
| 514 | 0.080 | NM | 0.003 | ME VAN | 2,2-DIMETHYL-1,3-PROPANEDIAMINE | 3-AC-BA |
| 515 | 0.019 | 0.031 | 0.011 | ME VAN | 2,2-DIMETHYL-1,3-PROPANEDIAMINE | 4-AC-CA |
| 516 | 0.030 | 0.031 | 0.001 | ME VAN | 2,2-DIMETHYL-1,3-PROPANEDIAMINE | 2-AC-CA |
| 517 | 0.017 | 0.03 | 0.013 | ME VAN | 2,2-DIMETHYL-1,3-PROPANEDIAMINE | AC SA |
| 518 | <.005 | 0.001 | 0.019 | ME VAN | 2,2-DIMETHYL-1,3-PROPANEDIAMINE | 6-AC-2-NA |
| 519 | 0.054 | 0.029 | 0.014 | ME VAN | ETHYLENEDIAMINE | 4-AC-BA |
| 520 | 0.135 | 0.024 | 0.008 | ME VAN | ETHYLENEDIAMINE | 3-AC-BA |
| 521 | 0.197 | 0.025 | 0.053 | ME VAN | ETHYLENEDIAMINE | 4-AC-CA |
| 522 | 0.008 | 0.024 | 0.002 | ME VAN | ETHYLENEDIAMINE | 2-AC-CA |
| 523 | 0.052 | 0.037 | 0.009 | ME VAN | ETHYLENEDIAMINE | AC SA |
| 524 | <.005 | 0.017 | 0.010 | ME VAN | ETHYLENEDIAMINE | 6-AC-2-NA |
| 525 | 0.019 | NM | 0.006 | ME VAN | CIS-1,2-DIAMINOCYCLOHEXANE | 4-AC-BA |
| 526 | 0.011 | 0.046 | 0.008 | ME VAN | CIS-1,2-DIAMINOCYCLOHEXANE | 3-AC-BA |
| 527 | 0.039 | 0.089 | 0.008 | ME VAN | CIS-1,2-DIAMINOCYCLOHEXANE | 4-AC-CA |
| 528 | 0.011 | >.05 | 0.001 | ME VAN | CIS-1,2-DIAMINOCYCLOHEXANE | 2-AC-CA |
| 529 | 0.011 | 0.079 | 0.020 | ME VAN | CIS-1,2-DIAMINOCYCLOHEXANE | AC SA |
| 530 | <.005 | RT | 0.010 | ME VAN | CIS-1,2-DIAMINOCYCLOHEXANE | 6-AC-2-NA |
| 531 | NM | 0.087 | 0.015 | ME VAN | 1,4-DIAMINOBUTANE | 4-AC-BA |
| 532 | 0.429 | 0.074 | 0.002 | ME VAN | 1,4-DIAMINOBUTANE | 3-AC-BA |
| 533 | 0.105 | 0.066 | 0.027 | ME VAN | 1,4-DIAMINOBUTANE | 4-AC-CA |
| 534 | 0.037 | 0.066 | <.005 | ME VAN | 1,4-DIAMINOBUTANE | 2-AC-CA |
| 535 | NM | >.05 | 0.009 | ME VAN | 1,4-DIAMINOBUTANE | AC SA |
| 536 | 0.004 | 0.013 | 0.026 | ME VAN | 1,4-DIAMINOBUTANE | 6-AC-2-NA |
| 537 | 0.015 | 0.028 | 0.012 | ME VAN | TRANS-1,4-DIAMINOCYCLOHEXANE | 4-AC-BA |
| 538 | 0.018 | 0.031 | 0.006 | ME VAN | TRANS-1,4-DIAMINOCYCLOHEXANE | 3-AC-BA |
| 539 | <.005 | 0.009 | 0.013 | ME VAN | TRANS-1,4-DIAMINOCYCLOHEXANE | 4-AC-CA |
| 540 | 0.018 | 0.017 | 0.003 | ME VAN | TRANS-1,4-DIAMINOCYCLOHEXANE | 2-AC-CA |
| 541 | 0.031 | 0.019 | 0.005 | ME VAN | TRANS-1,4-DIAMINOCYCLOHEXANE | AC SA |
| 542 | 0.004 | 0.007 | 0.008 | ME VAN | TRANS-1,4-DIAMINOCYCLOHEXANE | 6-AC-2-NA |
| 543 | NM | 0.195 | NM | ME VAN | 1,5-DIAMINOPENTANE | 4-AC-BA |
| 544 | 0.040 | 0.089 | 0.005 | ME VAN | 1,5-DIAMINOPENTANE | 3-AC-BA |
| 545 | 0.025 | 0.043 | 0.011 | ME VAN | 1,5-DIAMINOPENTANE | 4-AC-CA |
| 546 | 0.008 | 0.037 | <.005 | ME VAN | 1,5-DIAMINOPENTANE | 2-AC-CA |
| 547 | 0.017 | 0.062 | 0.018 | ME VAN | 1,5-DIAMINOPENTANE | AC SA |
| 548 | <.005 | 0.007 | 0.026 | ME VAN | 1,5-DIAMINOPENTANE | 6-AC-2-NA |
| 549 | 0.048 | 0.071 | 0.010 | ME VAN | 2,2′-THIOBIS(ETHYLAMINE) | 4-AC-BA |
| 550 | 0.038 | 0.058 | 0.008 | ME VAN | 2,2′-THIOBIS(ETHYLAMINE) | 3-AC-BA |
| 551 | NM | 0.283 | 0.008 | ME VAN | 2,2′-THIOBIS(ETHYLAMINE) | 4-AC-CA |
| 552 | 0.066 | NM | <.005 | ME VAN | 2,2′-THIOBIS(ETHYLAMINE) | 2-AC-CA |
| 553 | 0.060 | 0.108 | 0.016 | ME VAN | 2,2′-THIOBIS(ETHYLAMINE) | AC SA |
| 554 | 0.007 | 0.016 | 0.008 | ME VAN | 2,2′-THIOBIS(ETHYLAMINE) | 6-AC-2-NA |
| 555 | 0.026 | NM | 0.006 | ME VAN | N,N-BIS(3-AMINOPROPYL)METHYLAMINE | 4-AC-BA |
| 556 | 0.024 | NM | 0.004 | ME VAN | N,N-BIS(3-AMINOPROPYL)METHYLAMINE | 3-AC-BA |
| 557 | 0.005 | 0.009 | 0.008 | ME VAN | N,N-BIS(3-AMINOPROPYL)METHYLAMINE | 4-AC-CA |
| 558 | 0.015 | 0.016 | <.005 | ME VAN | N,N-BIS(3-AMINOPROPYL)METHYLAMINE | 2-AC-CA |
| 559 | 0.032 | 0.047 | 0.013 | ME VAN | N,N-BIS(3-AMINOPROPYL)METHYLAMINE | AC SA |
| 560 | 0.002 | 0.003 | 0.013 | ME VAN | N,N-BIS(3-AMINOPROPYL)METHYLAMINE | 6-AC-2-NA |
| 561 | 0.058 | 0.098 | 0.011 | ME VAN | HOMOPIPERAZINE | 4-AC-BA |
| 562 | 0.155 | NM | 0.011 | ME VAN | HOMOPIPERAZINE | 3-AC-BA |
| 563 | 0.021 | 0.316 | 0.015 | ME VAN | HOMOPIPERAZINE | 4-AC-CA |
| 564 | 0.017 | 0.033 | 0.010 | ME VAN | HOMOPIPERAZINE | 2-AC-CA |
| 565 | NM | 0.244 | 0.011 | ME VAN | HOMOPIPERAZINE | AC SA |
| 566 | <.005 | 0.007 | 0.015 | ME VAN | HOMOPIPERAZINE | 6-AC-2-NA |
| 567 | 0.040 | NM | 0.009 | ME VAN | 1,6-HEXANEDIAMINE | 4-AC-BA |
| 568 | <.005 | 0.173 | 0.004 | ME VAN | 1,6-HEXANEDIAMINE | 3-AC-BA |
| 569 | <.005 | NM | 0.022 | ME VAN | 1,6-HEXANEDIAMINE | 4-AC-CA |
| 570 | <.005 | NM | <.005 | ME VAN | 1,6-HEXANEDIAMINE | 2-AC-CA |
| 571 | <.005 | 0.097 | 0.016 | ME VAN | 1,6-HEXANEDIAMINE | AC SA |
| 572 | <.005 | 0.009 | 0.024 | ME VAN | 1,6-HEXANEDIAMINE | 6-AC-2-NA |
| 573 | 0.105 | NM | 0.025 | ME VAN | N,N′-DIMETHYLETHYLENEDIAMINE | 4-AC-BA |
| 574 | 0.065 | 0.137 | 0.011 | ME VAN | N,N′-DIMETHYLETHYLENEDIAMINE | 3-AC-BA |
| 575 | 0.029 | NM | 0.030 | ME VAN | N,N′-DIMETHYLETHYLENEDIAMINE | 4-AC-CA |
| 576 | 0.018 | NM | 0.018 | ME VAN | N,N′-DIMETHYLETHYLENEDIAMINE | 2-AC-CA |
| 577 | 0.062 | NM | 0.012 | ME VAN | N,N′-DIMETHYLETHYLENEDIAMINE | AC SA |
| 578 | <.005 | 0.017 | 0.026 | ME VAN | N,N′-DIMETHYLETHYLENEDIAMINE | 6-AC-2-NA |
| 579 | 0.069 | 0.095 | 0.007 | ME VAN | N,N′-DIETHYL-1,3-PROPANEDIAMINE | 4-AC-BA |
| 580 | 0.035 | 0.043 | 0.006 | ME VAN | N,N′-DIETHYL-1,3-PROPANEDIAMINE | 3-AC-BA |
| 581 | 0.015 | NM | 0.012 | ME VAN | N,N′-DIETHYL-1,3-PROPANEDIAMINE | 4-AC-CA |
| 582 | RT | NM | 0.003 | ME VAN | N,N′-DIETHYL-1,3-PROPANEDIAMINE | 2-AC-CA |
| 583 | NM | 0.242 | 0.057 | ME VAN | N,N′-DIETHYL-1,3-PROPANEDIAMINE | AC SA |
| 584 | <.005 | 0.026 | 0.013 | ME VAN | N,N′-DIETHYL-1,3-PROPANEDIAMINE | 6-AC-2-NA |
| 585 | 0.064 | 0.59 | 0.006 | ME VAN | 1,8-DIAMINO-3,6-DIOXAOCTANE | 4-AC-BA |
| 586 | 0.054 | NM | 0.003 | ME VAN | 1,8-DIAMINO-3,6-DIOXAOCTANE | 3-AC-BA |
| 587 | 0.039 | NM | 0.006 | ME VAN | 1,8-DIAMINO-3,6-DIOXAOCTANE | 4-AC-CA |
| 588 | 0.014 | NM | 0.008 | ME VAN | 1,8-DIAMINO-3,6-DIOXAOCTANE | 2-AC-CA |
| 589 | 0.112 | NM | 0.010 | ME VAN | 1,8-DIAMINO-3,6-DIOXAOCTANE | AC SA |
| 590 | <.005 | 0.028 | 0.044 | ME VAN | 1,8-DIAMINO-3,6-DIOXAOCTANE | 6-AC-2-NA |
| 591 | 0.104 | NM | 0.045 | ME VAN | N,N′-DIETHYL-2-BUTENE-1,4-DIAMINE | 4-AC-BA |
| 592 | 0.050 | NM | 0.002 | ME VAN | N,N′-DIETHYL-2-BUTENE-1,4-DIAMINE | 3-AC-BA |
| 593 | 0.007 | NM | 0.001 | ME VAN | N,N′-DIETHYL-2-BUTENE-1,4-DIAMINE | 4-AC-CA |
| 594 | 0.009 | NM | 0.022 | ME VAN | N,N′-DIETHYL-2-BUTENE-1,4-DIAMINE | 2-AC-CA |
| 595 | NM | 0.171 | 0.039 | ME VAN | N,N′-DIETHYL-2-BUTENE-1,4-DIAMINE | AC SA |
| 596 | <.005 | 0.005 | 0.003 | ME VAN | N,N′-DIETHYL-2-BUTENE-1,4-DIAMINE | 6-AC-2-NA |
| 597 | 0.108 | NM | 0.006 | ME-P-OH BN | PIPERAZINE | 4-AC-BA |
| 598 | 0.127 | NM | 0.006 | ME-P-OH BN | PIPERAZINE | 3-AC-BA |
| 599 | 0.112 | 0.061 | 0.014 | ME-3-OH BN | PIPERAZINE | 4-AC-BA |
| 600 | 0.043 | 0.049 | 0.053 | ME-P-OH BN | PIPERAZINE | 4-AC-CA |
| 601 | 0.024 | 0.022 | 0.011 | ME-P-OH BN | PIPERAZINE | 2-AC-CA |
| 602 | 0.027 | 0.03 | 0.018 | ME-P-OH BN | PIPERAZINE | 4-AC-3-Ome-CA |
| 603 | 0.032 | 0.096 | 0.003 | ME-P-OH BN | PIPERAZINE | N,O-DIACTYR |
| 604 | 0.200 | 0.04 | 0.002 | ME-3-CL-4-OH BN | PIPERAZINE | 4-AC-BA |
| 605 | 0.107 | 0.399 | 0.004 | ME-3-CL-4-OH BN | PIPERAZINE | 3-AC-BA |
| 606 | 0.074 | 0.025 | 0.012 | ME-3-CL-4-OH BN | PIPERAZINE | 4-AC-CA |
| 607 | 0.006 | 0.009 | 0.002 | ME-3-CL-4-OH BN | PIPERAZINE | 2-AC-CA |
| 608 | <.005 | 0.019 | 0.004 | ME-3-CL-4-OH BN | PIPERAZINE | 4-AC-3-Ome-CA |
| 609 | 0.147 | 0.064 | 0.017 | ME-3-CL-4-OH BN | PIPERAZINE | N,O-DIACTYR |
| 610 | 0.513 | 0.137 | 0.039 | ME-3-OH BN | PIPERAZINE | 3-AC-BA |
| 611 | 0.015 | 0.029 | 0.000 | ME-3-OH BN | PIPERAZINE | 4-AC-CA |
| 612 | 0.042 | 0.024 | 0.002 | ME-3-OH BN | PIPERAZINE | 2-AC-CA |
| 613 | 0.018 | 0.043 | 0.006 | ME-3-OH BN | PIPERAZINE | 4-AC-3-Ome-CA |
| 614 | 0.058 | 0.109 | 0.053 | ME-3-OH BN | PIPERAZINE | N,O-DIACTYR |
| 615 | 0.053 | 0.034 | 0.011 | MEVAN | PIPERAZINE | 4-AC-BA |
| 616 | 0.056 | 0.048 | 0.018 | ME VAN | PIPERAZINE | 3-AC-BA |
| 617 | 0.011 | 0.018 | 0.001 | ME VAN | PIPERAZINE | 4-AC-CA |
| 618 | 0.011 | 0.014 | 0.002 | ME VAN | PIPERAZINE | 2-AC-CA |
| 619 | 0.026 | 0.048 | 0.002 | ME VAN | PIPERAZINE | 4-AC-3-OMe-CA |
| 620 | 0.063 | NM | 0.012 | ME VAN | PIPERAZINE | N,O-DIACTYR |

We claim:

1. A compound represented by formula 1a: 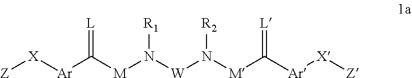
wherein, independently for each occurrence,
X and X′ represent independently O or S;
Z and Z′ each represent independently H, or optionally substituted alkyl, alkenyl, alkynyl, aryl, heteroaryl or acyl;
L and L′ each independently represent O, NR″, or S;
M and M′ independently represent a bond, or a bivalent alkyl or alkenyl chain.
W represents an optionally substituted bivalent alkyl, alkenyl, alkynyl, cycloalkyl, cylcolalkenyl, aryl or heteroaryl group, wherein the alkyl, alkenyl or alkynyl groups optionally contain one or more heteroatoms selected from O, S, or NR″′;
R₁ and R₂ represent H, or optionally substituted alkyl, alkenyl, alkynyl, aralkyl, cycloalkyl, aryl or heteroaryl; or R₁ and R₂ may be joined together to form an optionally substituted 4 to 8 membered heterocyclic ring, wherein the ring includes W and the nitrogens to which R₁ and R₂ are attached, provided that the heterocyclic ring is not unsubstituted piperazine, and provided that when R₁ and R₂ are both H, W is not bivalent n-propyl;
Ar and Ar′ independently represent optionally substituted aryl or heteroaryl;
R′, R″ and R″′ represent independently for each occurrence H, formyl, sulfonyl, or —(CH₂)ₘ—R₈₀, or optionally substituted alkyl, alkenyl, aryl, aralkyl, formyl or acyl;
R₈₀ represents independently for each occurrence optionally substituted aryl, cycloalkyl, cycloalkenyl, or heterocyclyl; and
or a pharmaceutically acceptable salt thereof.

2. The compound of claim 1, wherein X and X′ each represent O.

3. The compound of claim 1, wherein Z and Z′ each represent H.

4. The compound of claim 1, wherein Z and Z′ each represent optionally substituted alkyl, alkenyl, alkynyl, aryl, heteroaryl or acyl.

5. The compound of claim 1, wherein Ar and Ar′ independently represent optionally substituted phenyl or naphthyl.

6. The compound of claim 5, wherein Ar and Ar′ are optionally substituted with at least one member selected from the group consisting of halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, and sulfonamido.

7. The compound of claim 6, wherein Ar and Ar′ are optionally substituted with at least one halogen or alkoxy group.

8. The compound of claim 1, wherein M and M′ are each a bond.

9. The compound of claim 9, wherein at least one of M and M′ is —CH═CH—.

10. The compound of claim 1, wherein L and L′ independently represent O or NR′.

11. The compound of claim 10, wherein L and L′ independently represent O or NH.

12. The compound of claim 11, wherein L and L′ independently represent O.

13. The compound of claim 1, wherein W represents a bivalent alkyl or cycloalkyl group, wherein the alkyl group optionally contains one or more heteroatoms selected from O, S, or NR″′.

14. The compound of claim 13, wherein R₁ and R₂ independently represent H, or optionally substituted alkyl, or R₁ and R₂ may be joined together to form an optionally substituted 4 to 8 membered heterocyclic ring, wherein the ring includes W and the nitrogens to which R₁ and R₂ are attached;

15. The compound of claim 14, wherein R₁ and R₂ independently represent H, methyl, ethyl, or propyl.

16. The compound of claim 13, wherein W represents a bivalent cyclohexyl group.

17. The compound of claim 13, wherein W represents —(CH₂)ₙ—, wherein n is 1, 2, or an integer ranging from 4 to 12, inclusive.

18. The compound of claim 17, wherein n is 2, 4, 5, or 6.

19. The compound of claim 13, wherein W represents a bivalent alkyl containing one or more heteroatoms selected from O, S, and NR″′.

20. The compound of claim 19, wherein W represents a bivalent alkyl containing at least one S.

21. The compound of claim 19, wherein W represents a bivalent alkyl containing at least one O.

22. The compound of claim 19, wherein W represents a bivalent alkyl containing at least one NR″′.

23. The compound of claim 19, wherein W represents:
—(CH₂)₂—S(CH₂)₂—, —(CH₂)₂—O—(CH₂)₂—O—(CH₂)₂—, —(CH₂)₃—N(CH₃)—(CH₂)₃—, —CH₂CH═CHCH₂—, or —CH₂C(CH₃)₂CH₂—.

24. The compound of claim 14, wherein W, R₁ and R₂ together form a homopiperazine ring.

25. The compound of claim 1, wherein:
X and X′ are each O;
Z and Z′ are each H;
L and L′ are each O;
M and M′ are each a bond; and
Ar and Ar′ are each phenyl or napthyl;

26. A compound represented by formula 1b: 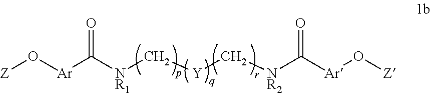
wherein, independently for each occurrence:
Y represents CH₂ or NR′;
R₁ and R₂ independently represent H, or optionally substituted alkyl or aryl, or R₁ and R₂ are joined together to form a optionally substituted 4 to 8 membered heterocyclic ring, wherein the ring includes —(CH₂)ₚ(Y)_{q}(CH₂)ᵣ— and the nitrogens to which R₁ and R₂ are attached; provided that the heterocyclic ring is not unsubstituted piperazine, and provided that when R₁ and R₂ are both H, W is not bivalent n-propyl;
p and r are 1, 2, or 3;
q is 0 or 1; and
Ar and Ar′ represent optionally substituted phenyl or substituted naphthyl;
Z and Z′ represent independently H or optionally substituted alkyl, alkenyl, alkynyl, aryl, heteroaryl or acyl;
or a pharmaceutically acceptable salt thereof.

27. The compound of claim 26, wherein, wherein R₁ and R₂ independently represent H or alkyl.

28. The compound of claim 26, wherein p and r are each 3.

29. The compound of claim 26, wherein q is 1 and Y is NMe.

30. The compound of claim 26, wherein Ar and Ar′ are each optionally substituted with at least one member selected from the group consisting of halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, and sulfonamido.

31. The compound of claim 30, wherein Ar and Ar′ are optionally substituted with halogen or alkoxy.

32. The compound of claim 1, having the formula:

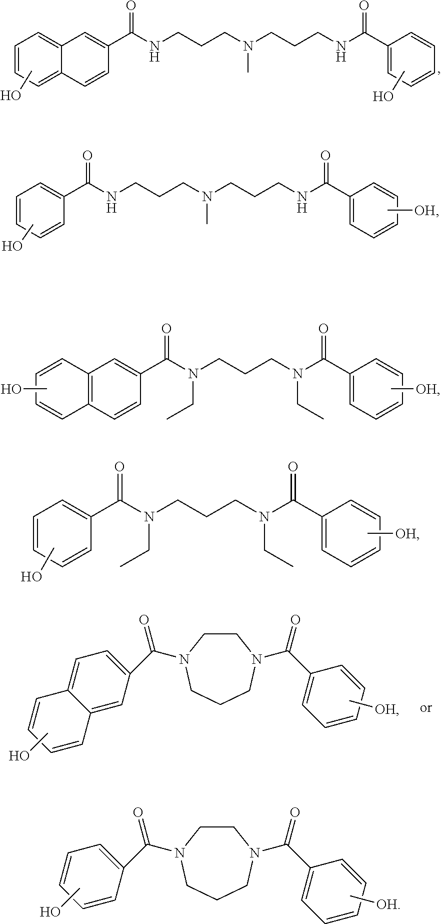

33. The compound of claim 32, having the formula:

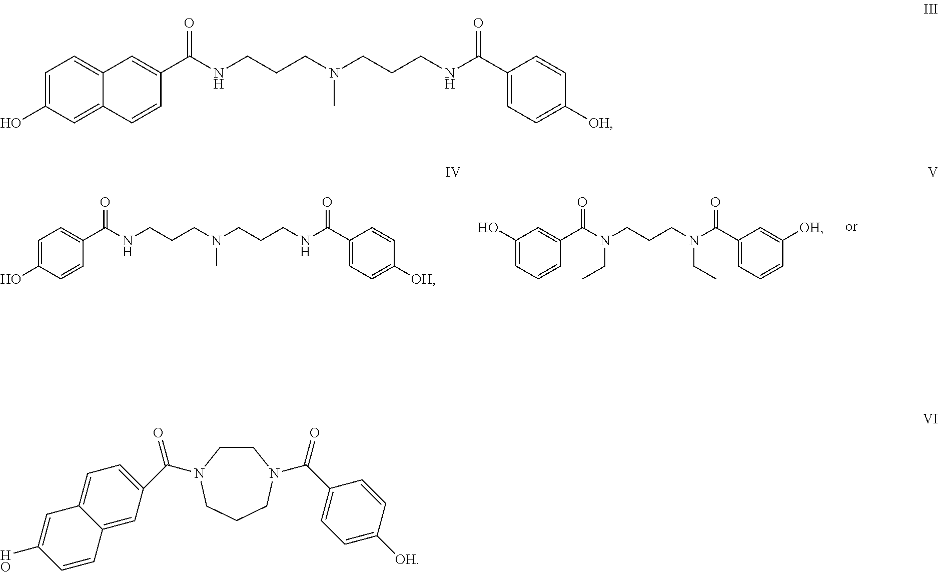

34. A composition comprising a compound of any one of claim 1 and a pharmaceutically acceptable carrier.
